(19) United States
(12) Patent Application Publication
(10) Pub. No.: US 2021/0196699 A1
(43) Pub. Date: Jul. 1, 2021

(54) CGRP ANTAGONISTS FOR TREATING MIGRAINE BREAKTHROUGH

(71) Applicant: Biohaven Pharmaceutical Holding Company Ltd., New Haven, CT (US)

(72) Inventors: Vladimir Coric, Madison, CT (US); Robert Croop, Newton Square, PA (US)

(21) Appl. No.: 17/178,217

(22) Filed: Feb. 17, 2021

Related U.S. Application Data
(63) Continuation of application No. 17/269,227.
(60) Provisional application No. 62/794,665, filed on Jan. 20, 2019.
(60) Provisional application No. 62/844,169, filed on May 7, 2019.
(60) Provisional application No. 62/893,206, filed on Aug. 29, 2019.
(60) Provisional application No. 62/910,284, filed on Oct. 3, 2019.
(60) Provisional application No. 62/959,088, filed on Jan. 9, 2020.

(51) Int. Cl. A61K 31/4545; A61K 9/00; A61P 25/06; A61K 31/4709

(52) U.S. Cl. CPC A61K 31/4545; A61K 31/4709; A61P 25/06; A61K 9/0043

(57) ABSTRACT

Disclosed are methods of treating breakthrough migraine in patients using breakthrough CGRP antagonists. Also disclosed are methods for the prophylactic treatment of migraine.

### CROSS-REFERENCE TO RELATED APPLICATIONS

This application is a continuation of U.S. patent application Ser. No. 17/269,227 filed Feb. 17, 2021, which is a national stage application under 35 U.S.C. § 371 of International Application No. PCT/US2020/014239 filed Jan. 19, 2020, which claims priority to and the benefit of U.S. Provisional Application No. 62/794,665 filed Jan. 20, 2019, U.S. Provisional Application No. 62/844,169 filed May 7, 2019, U.S. Provisional Application No. 62/893,206 filed Aug. 29, 2019, U.S. Provisional Application No. 62/910,284 filed Oct. 3, 2019, and U.S. Provisional Application No. 62/959,088 filed Jan. 9, 2020, the contents of all of the above applications are incorporated herein in their entireties by reference.

### FIELD OF THE INVENTION

The present invention relates to the treatment of patients that undergo migraine breakthrough while being treated with migraine medications.

### BACKGROUND OF THE INVENTION

Migraine is a chronic and debilitating disorder characterized by recurrent attacks lasting four to 72 hours with multiple symptoms, including typically one-sided, pulsating headaches of moderate to severe pain intensity that are associated with nausea or vomiting, and/or sensitivity to sound (phonophobia) and sensitivity to light (photophobia). Migraines are often preceded by transient neurological warning symptoms, known as auras, which typically involve visual disturbances such as flashing lights, but may also involve numbness or tingling in parts of the body. Migraine is both widespread and disabling. The Migraine Research Foundation ranks migraine as the world's third most prevalent illness, and the Global Burden of Disease Study 2015 rates migraine as the seventh highest specific cause of disability worldwide. According to the Migraine Research Foundation, in the United States, approximately 36 million individuals suffer from migraine attacks. While most sufferers experience migraine attacks once or twice per month, more than 4 million people have chronic migraine, defined as experiencing at least 15 headache days per month, of which at least eight are migraine, for more than three months. Others have episodic migraine, which is characterized by experiencing less than 15 migraine days per month. People with episodic migraine may progress to chronic migraine over time. Migraine attacks can last four hours or up to three days. More than 90% of individuals suffering from migraine attacks are unable to work or function normally during a migraine attack, with many experiencing comorbid conditions such as depression, anxiety and insomnia. Also, those suffering from migraine often have accompanying nausea and have an aversion to consuming food or liquids during an attack.

CGRP (calcitonin gene-related peptide) is a 37 amino acid neuropeptide, which belongs to a family of peptides that includes calcitonin, adrenomedullin and amylin. In humans, two forms of CGRP (a-CGRP and 13-CGRP) exist and have similar activities. They vary by three amino acids and exhibit differential distribution. At least two CGRP receptor subtypes may also account for differential activities. The CGRP receptor is located within pain-signaling pathways, intracranial arteries and mast cells and its activation is thought to play a causal role in migraine pathophysiology. For example, research and clinical studies have shown: serum levels of CGRP are elevated during migraine attacks, infusion of intravenous CGRP produces persistent pain in migraine sufferers and non-migraine sufferers, and treatment with anti-migraine drugs normalizes CGRP activity.

Possible CGRP involvement in migraine has been the basis for the development and clinical testing of a number of compounds, including for example, olcegepant (Boehringer Ingelheim, Ridgefield, Conn.), telcagepant (Merck Sharp & Dohme Corp., Kenilworth, N.J.), ubrogepant and atogepant (Allergan plc, Dublin, Ireland), lasmiditan (Eli Lilly and Company, Indianapolis, Ind.), rimegepant (Biohaven Pharmaceutical Holding Company Ltd., New Haven, Conn.), galcanezumab (Eli Lilly and Company, Indianapolis, Ind.), fremanezumab (Teva Pharmaceutical Industries, Petah Tikva, Israel), eptinezumab (Alder Biopharmaceuticals, Inc., Bothell, Wash.), and erenumab (Amgen Inc., Thousand Oaks, Calif.). Currently, clinicians use a number of pharmacologic agents for the acute treatment of migraine. A study published by the American Headache Society in 2015 concluded that the medications deemed effective for the acute treatment of migraine fell into the following classes: triptans, ergotamine derivatives, non-steroidal anti-inflammatory drugs (“NSAIDs”), opioids and combination medications. The current standard of care for the acute treatment of migraine is prescription of triptans, which are serotonin 5-HT_{1B/1D} receptor agonists. Triptans have been developed and approved for the acute treatment of migraine over the past two decades. The initial introduction of triptans represented a shift toward drugs more selectively targeting the suspected pathophysiology of migraine. While triptans account for almost 80% of anti-migraine therapies prescribed at office visits by healthcare providers, issues such as an incomplete effect or headache recurrence remain important clinical limitations. In fact, only about 30% of patients from clinical trials are pain free at two hours after taking triptans. In addition, triptans are contraindicated in patients with cardiovascular disease, cerebrovascular disease, or significant risk factors for either because of potential systemic and cerebrovascular vasoconstriction from the 5-HT_{1B}-mediated effects. Also, according to a January 2017 study published in the journal Headache, an estimated 2.6 million migraine sufferers in the United States have a cardiovascular event, condition or procedure that limits the potential of triptans as a treatment option.

Some patients being treated with migraine medications, for example biologic medications such as antibodies, e.g., galcanezumab, fremanezumab, eptinezumab or erenumab, may experience breakthrough of migraine headaches, symptoms or episodes despite being treated with their current migraine medications.

Accordingly, there remains a significant unmet medical need for a novel migraine-specific medication that provides enhanced patient benefits compared to existing therapies. More specifically, therapies are desired to treat patients being treated for migraine that undergo breakthrough of migraine headaches, symptoms or episodes.

### SUMMARY OF THE INVENTION

The present invention is directed to the treatment of patients undergoing treatment for migraine that undergo breakthrough of migraine headaches, symptoms or episodes. By virtue of the present invention, it may now be possible to provide more effective GCRP related treatments to patients. Patients suffering from migraine may experience an improved response in one or more areas including, for example, pain freedom or freedom from most bothersome symptoms.

In an aspect of the invention, there is provided a method of treating breakthrough migraine in a patient undergoing underlying treatment with a migraine medication who has experienced a breakthrough resulting in a migraine headache, symptom or episode, said method including administering to the patient a pharmaceutical composition including a therapeutically effective amount of a breakthrough CGRP antagonist, or a pharmaceutically acceptable salt thereof.

In another aspect, the migraine medication used in the underlying treatment may be a biologic.

In another aspect, the biologic may be an antibody.

In another aspect, the antibody may be selected from galcanezumab-gnlm, fremanezumab-vfrm, eptinezumab and erenumab-aooe.

In another aspect, the breakthrough CGRP antagonist may be a non-biologic CGRP antagonist.

In another aspect, the breakthrough CGRP antagonist may not include an antibody, an antibody fragment, or a peptide.

In another aspect, the breakthrough CGRP antagonist may be selected from olcegepant, telcagepant, ubrogepant, atogepant, rimegepant, and zavegepant.

In another aspect, the breakthrough CGRP antagonist may be selected from ubrogepant, rimegepant, and zavegepant.

In another aspect, the breakthrough CGRP antagonist may be ubrogepant or atogepant.

In another aspect, the breakthrough CGRP antagonist may be rimegepant.

In another aspect, the breakthrough CGRP antagonist may be zavegepant.

In another aspect, the antibody may be galcanezumab-gnlm, and the breakthrough CGRP antagonist may be ubrogepant or atogepant.

In another aspect, the antibody may be galcanezumab-gnlm, and the breakthrough CGRP antagonist may be rimegepant.

In another aspect, the antibody may be galcanezumab-gnlm, and the breakthrough CGRP antagonist may be zavegepant.

In another aspect, the antibody may be fremanezumab-vfrm, and the breakthrough CGRP antagonist may be ubrogepant or atogepant.

In another aspect, the antibody may be fremanezumab-vfrm, and the breakthrough CGRP antagonist may be rimegepant.

In another aspect, the antibody may be fremanezumab-vfrm, and the breakthrough CGRP antagonist may be zavegepant.

In another aspect, the antibody may be eptinezumab, and the breakthrough CGRP antagonist may be ubrogepant or atogepant.

In another aspect, the antibody may be eptinezumab, and the breakthrough CGRP antagonist may be rimegepant.

In another aspect, the antibody may be eptinezumab, and the breakthrough CGRP antagonist may be zavegepant.

In another aspect, the antibody may be erenumab-aooe, and the breakthrough CGRP antagonist may be ubrogepant or atogepant.

In another aspect, the antibody may be erenumab-aooe, and the breakthrough CGRP antagonist may be rimegepant.

In another aspect, the antibody may be erenumab-aooe, and the breakthrough CGRP antagonist may be zavegepant.

In another aspect, the migraine medication used in the underlying treatment may include a triptan and an antibody.

In another aspect, the triptan may be selected from rizatriptan, sumatriptan, naratriptan, eletriptan, donitriptan, almotriptan, frovatriptan, avitriptan, and zolmitriptan.

In another aspect, the triptan may be other than rizatriptan, sumatriptan, naratriptan, eletriptan, donitriptan, almotriptan, frovatriptan, avitriptan, or zolmitriptan.

In another aspect, the underlying treatment may be a treatment with at least one non-triptan drug.

In another aspect, the underlying treatment may take place for one week, two weeks, three weeks, four weeks, five weeks, six weeks, seven weeks, eight weeks, nine weeks, ten weeks, eleven weeks, or twelve or more weeks.

In another aspect, the patient may experience reduced frequency or reduced severity of migraine after treatment with the CGRP antagonist.

In another aspect, the migraine headache, symptom, or episode may be selected from sinusitis, nausea, nasopharangytis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light (photophobia), sounds (phonophobia), or smells, worsening pain during physical activity, and vomiting, abdominal pain or heartburn, loss of appetite, lightheadedness, blurred vision, and fainting.

In another aspect, the migraine headache, symptom, or episode may be present after the treatment with at least one triptan drug and at least one antibody.

In another aspect, the migraine headache, symptom, or episode may be reduced after treatment with the CGRP antagonist.

In another aspect, the CGRP antagonist may be administered at a dose of about 1-1000 mg per day.

In another aspect, the CGRP antagonist may be administered at a dose of about 1, 5, 10, 15, 20, 25, 30, 40, 50, 60, 70, 80, 90, 100, 200, 250, 300, 400, 500, 750, or 1000 mg per day.

In another aspect, the CGRP antagonist may be administered orally.

In another aspect, the CGRP antagonist may be administered intranasally.

In another aspect, the pharmaceutical composition may include about 50 mg or 100 mg of ubrogepant or a pharmaceutically acceptable salt thereof equivalent in potency to about 50 mg or 100 mg of ubrogepant.

In another aspect, the pharmaceutical composition may include about 75 mg of rimegepant or a pharmaceutically acceptable salt thereof equivalent in potency to about 75 mg of rimegepant.

In another aspect, the rimegepant may be in the form of a hemisulfate sesquihydrate salt.

In another aspect, the pharmaceutical composition may be in the form of a tablet.

In another aspect, the pharmaceutical composition may be in the form of a capsule.

In another aspect, the pharmaceutical composition may include about 50-60 weight % rimegepant hemisulfate sesquihydrate, about 30-35 weight % microcrystalline cellulose, about 2-7 weight % hydroxypropyl cellulose, about 3-7 weight % croscarmellose sodium, and about 0.1-1.0 weight % magnesium stearate.

In another aspect, the pharmaceutical composition may include about 57.1 weight % rimegepant hemisulfate sesquihydrate, about 33.4 weight % microcrystalline cellulose, about 4.0 weight % hydroxypropyl cellulose, about 5.0 weight % croscarmellose sodium, and about 0.5 weight % magnesium stearate.

In another aspect, the pharmaceutical composition may be in the form of an oral solid molded fast-dispersing dosage form.

In another aspect, the pharmaceutical composition may include from about 70-80 weight % rimegepant hemisulfate sesquihydrate, about 10-20 weight % fish gelatin, about 10-20 weight % of a filler, and 0.1-5.0 weight % of a flavorant.

In another aspect, the filler may be mannitol.

In another aspect, the pharmaceutical composition may be a spayed-dried composition.

In another aspect, the sprayed-dried composition may include hypermellose succinate acetate and a therapeutically effective amount of a breakthrough CGRP antagonist, or a pharmaceutically acceptable salt thereof.

In another aspect, the biologic may be a neurotoxic protein.

In another aspect, the neurotoxic protein may be botulinum toxin.

In another aspect, the breakthrough CGRP antagonist may be selected from olcegepant, telcagepant, ubrogepant, atogepant, rimegepant, and zavegepant.

In another aspect, the breakthrough CGRP antagonist may be rimegepant.

In another aspect, the rimegepant may be in the form of a hemisulfate sesquihydrate salt.

In another aspect, the pharmaceutical composition may include about 50-60 weight % rimegepant hemisulfate sesquihydrate, about 30-35 weight % microcrystalline cellulose, about 2-7 weight % hydroxypropyl cellulose, about 3-7 weight % croscarmellose sodium, and about 0.1-1.0 weight % magnesium stearate.

In another aspect, the pharmaceutical composition may include about 57.1 weight % rimegepant hemisulfate sesquihydrate, about 33.4 weight % microcrystalline cellulose, about 4.0 weight % hydroxypropyl cellulose, about 5.0 weight % croscarmellose sodium, and about 0.5 weight % magnesium stearate.

In another aspect, the pharmaceutical composition may be in the form of an oral solid molded fast-dispersing dosage form.

In another aspect, the pharmaceutical composition may include from about 70-80 weight % rimegepant hemisulfate sesquihydrate, about 10-20 weight % fish gelatin, about 10-20 weight % of a filler, and 0.1-5.0 weight % of a flavorant.

In another aspect, the filler may be mannitol.

In another aspect, the biologic may be a neurotoxic protein and an antibody.

In another aspect, the neurotoxic protein may be botulinum toxin, and the antibody may be selected from galcanezumab-gnlm, fremanezumab-vfrm, eptinezumab and erenumab-aooe.

In another aspect, the breakthrough CGRP antagonist may be selected from olcegepant, telcagepant, ubrogepant, atogepant, rimegepant, and zavegepant.

In another aspect, the breakthrough CGRP antagonist may be rimegepant.

In another aspect, the rimegepant may be in the form of a hemisulfate sesquihydrate salt.

In another aspect, the pharmaceutical composition may include about 50-60 weight % rimegepant hemisulfate sesquihydrate, about 30-35 weight % microcrystalline cellulose, about 2-7 weight % hydroxypropyl cellulose, about 3-7 weight % croscarmellose sodium, and about 0.1-1.0 weight % magnesium stearate.

In another aspect, the pharmaceutical composition may include about 57.1 weight % rimegepant hemisulfate sesquihydrate, about 33.4 weight % microcrystalline cellulose, about 4.0 weight % hydroxypropyl cellulose, about 5.0 weight % croscarmellose sodium, and about 0.5 weight % magnesium stearate.

In another aspect, the pharmaceutical composition may be in the form of an oral solid molded fast-dispersing dosage form.

In another aspect, the pharmaceutical composition may include from about 70-80 weight % rimegepant hemisulfate sesquihydrate, about 10-20 weight % fish gelatin, about 10-20 weight % of a filler, and 0.1-5.0 weight % of a flavorant.

In another aspect, the filler may be mannitol.

### DETAILED DESCRIPTION OF THE INVENTION

The following detailed description is provided to aid those skilled in the art in practicing the present invention. Those of ordinary skill in the art may make modifications and variations in the embodiments described herein without departing from the spirit or scope of the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

The term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

As used herein, the term “administering” refers to the physical introduction of a composition comprising a therapeutic agent to a subject, using any of the various methods and delivery systems known to those skilled in the art. Administering can also be performed, for example, once, a plurality of times, and/or over one or more extended periods and can be a therapeutically effective dose or a subtherapeutic dose.

As used herein, the term “biologic” refers to molecules that are obtained or capable of being obtained through use of biological methods, commonly mammalian or plant cells, bacteria, insects, or yeast. The resultant products are commonly proteins, but may be nucleic acids, carbohydrates, or a combination of multiple types of molecules. Biologics commonly, but not necessarily, fall into general functional categories of monoclonal antibodies, cytokines, growth factors, enzymes, peptides, and proteins (which include neurotoxic proteins) that are focused on specific targets. In each category, the biologic may target the signaling or effector molecule or its receptor. Biologics are commonly large molecules having molecular weight of 100 kDaltons or greater, 110 kDaltons or greater, 120 kDaltons or greater, 130 kDaltons or greater, 140 kDaltons or greater, or 150 kDaltons or greater, but are not limited thereto. Biologics also include biosimilar molecules (or biosimilars), which are molecular entities that are structurally similar to and have no clinically meaningful differences in terms of safety, purity, and potency from known biologics.

As used herein, the term “antibody” (Ab) refers to, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. As used herein, the term “isotype” refers, without limitation, to the antibody class or subclass (e.g., IgM or IgG1) that is encoded by the heavy chain constant region genes. In certain embodiments, one or more amino acids of the isotype can be mutated to alter effector function. As used herein, the term “antibody” includes, by way of example, both naturally occurring and non-naturally occurring Abs; monoclonal and polyclonal Abs; chimeric and humanized Abs; human or nonhuman Abs; wholly synthetic Abs; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

As used herein, the terms “botulinum toxin” and “botulinum”, also referred to as “Botox” mean a neurotoxin produced by Clostridium botulinum, as well as a botulinum toxin, fragments, variants or chimeras thereof made recombinantly by a non-Clostridial species. The phrase “botulinum toxin”, as used herein, encompasses botulinum toxin serotype A (BoNT/A), botulinum toxin serotype B (BoNT/B), botulinum toxin serotype C (BoNT/C), botulinum toxin serotype D (BoNT/D), botulinum toxin serotype E (BoNT/E), botulinum toxin serotype F (BoNT/F), botulinum toxin serotype G (BoNT/G), botulinum toxin serotype H (BoNT/H), botulinum toxin serotype X (BoNT/X), botulinum toxin serotype En (BoNT/En), and mosaic botulinum toxins and/or their subtypes and any other types of subtypes thereof, or any re-engineered proteins, analogs, derivatives, homologs, parts, sub-parts, variants, or versions, in each case, of any of the foregoing. As used herein, “botulinum toxin”, also encompasses a “modified botulinum toxin”. Further “botulinum toxin” as used herein also encompasses a botulinum toxin complex, (for example, the 300, 600 and 900 kDa complexes), as well as the neurotoxic component of the botulinum toxin (150 kDa) that is unassociated with the complex proteins.

As used herein, the term “chimeric antibody” refers, without limitation, to an antibody in which the variable regions are derived from one species and the constant regions are derived from another species, such as an antibody in which the variable regions are derived from a mouse antibody and the constant regions are derived from a human antibody.

As used herein, the terms “in combination with” and “in conjunction with” refer to administration of one treatment modality in addition to another treatment modality. As such, “in combination with” or “in conjunction with” refers to administration of one treatment modality before, during, or after administration of the other treatment modality to the subject.

As used herein, the term “human antibody” (HuMAb) refers, without limitation, to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

As used herein, the term “humanized antibody” refers, without limitation, to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody. The terms “humanized” antibodies and “fully humanized” antibodies and are used synonymously.

As used herein, the term “monoclonal antibody” (“mAb”) refers, without limitation, to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

As used herein, the term “pharmaceutically acceptable salt” refers to a salt form of one or more of the compounds or prodrugs described herein which are presented to increase the solubility of the compound in the gastric or gastroenteric juices of the patient's gastrointestinal tract in order to promote dissolution and the bioavailability of the compounds. Pharmaceutically acceptable salts include those derived from pharmaceutically acceptable inorganic or organic bases and acids, where applicable. Suitable salts include those derived from alkali metals such as potassium and sodium, alkaline earth metals such as calcium, magnesium and ammonium salts, among numerous other acids and bases well known in the pharmaceutical art.

As used herein, the terms “subject” and “patient” refer any human or nonhuman animal. The term “nonhuman animal” includes, but is not limited to, vertebrates such as nonhuman primates, sheep, dogs, and rodents such as mice, rats and guinea pigs. In some embodiments, the subject is a human. The terms, “subject” and “patient” are used interchangeably herein.

As used herein, the terms “effective amount”, “therapeutically effective amount”, “therapeutically effective dosage” and “therapeutically effective dose” of an agent (also sometimes referred to herein as a “drug”) refers to any amount of the agent that, when used alone or in combination with another agent, protects a subject against the onset of a disease or promotes disease regression evidenced by a decrease in severity of disease symptoms, an increase in frequency and duration of disease symptom-free periods, or a prevention of impairment or disability due to the disease affliction. The therapeutically effective amount of an agent can be evaluated using a variety of methods known to the skilled practitioner, such as in human subjects during clinical trials, in animal model systems predictive of efficacy in humans, or by assaying the activity of the agent in in vitro assays.

As used herein, the term “treatment” refers to any treatment of a condition or disease in a subject and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. Treatment could be used in combination with other standard therapies or alone. Treatment or “therapy” of a subject also includes any type of intervention or process performed on, or the administration of an agent to, the subject with the objective of reversing, alleviating, ameliorating, inhibiting, slowing down or preventing the onset, progression, development, severity or recurrence of a symptom, complication or condition, or biochemical indicia associated with a disease.

With respect to headache, “treatment” is an approach for obtaining beneficial or desired clinical results. For purposes of this invention, beneficial or desired clinical results include, but are not limited to, one or more of the following: improvement in any aspect of a headache including lessening severity, alleviation of pain intensity, and other associated symptoms, reducing frequency of recurrence, increasing the quality of life of those suffering from the headache, and decreasing dose of other medications required to treat the headache. For migraine, other associated symptoms include, but are not limited to, nausea, vomiting, and sensitivity to light, sound, and/or movement. For cluster headache, other associated symptoms include, but are not limited to swelling under or around the eyes, excessive tears, red eye, Rhinorrhea or nasal congestion, and red flushed face.

As used herein, the term “reducing incidence” of headache means any of reducing severity (which can include reducing need for and/or amount of (e.g., exposure to) other drugs and/or therapies generally used for this condition, including, for example, ergotamine, dihydroergotamine, or triptans for migraine), duration, and/or frequency (including, for example, delaying or increasing time to next episodic attack in an individual). As is understood by those skilled in the art, individuals may vary in terms of their response to treatment, and, as such, for example, a “method of reducing incidence of headache in an individual” reflects administering the rimegepant based on a reasonable expectation that such administration may likely cause such a reduction in incidence in that particular individual.

As used herein, the term “ameliorating” headache or one or more symptoms of headache means a lessening or improvement of one or more symptoms of headache as compared to not administering a treatment. “Ameliorating” also includes shortening or reduction in duration of a symptom.

As used herein, the term “delaying” the development of headache means to defer, hinder, slow, retard, stabilize, and/or postpone progression of the disease. This delay can be of varying lengths of time, depending on the history of the disease and/or individuals being treated. As is evident to one skilled in the art, a sufficient or significant delay can, in effect, encompass prevention, in that the individual does not develop headache (e.g., migraine). A method that “delays” development of the symptom is a method that reduces probability of developing the symptom in a given time frame and/or reduces extent of the symptoms in a given time frame, when compared to not using the method. Such comparisons are typically based on clinical studies, using a statistically significant number of subjects.

As used herein, the term “development” or “progression” of headache means initial manifestations and/or ensuing progression of the disorder. Development of headache can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of headache includes initial onset and/or recurrence.

In an embodiment, provided is a method of treating breakthrough migraine in a patient undergoing underlying treatment with a migraine medication who has experienced a breakthrough resulting in a migraine headache, symptom or episode, wherein the method includes administering to the patient a pharmaceutical composition comprising a therapeutically effective amount of a breakthrough CGRP antagonist, or a pharmaceutically acceptable salt thereof.

As used herein, the term “breakthrough migraine” refers to a migraine headache, symptom or episode that takes place when a patient currently undergoes or previously underwent treatment with an anti-migraine medication. The migraine headache, symptom, or episode may include sinusitis, nausea, nasopharangytis, photophobia, appetite changes, cognition and concentration difficulties, cold extremities, diarrhea or other bowel changes, excitement or irritability, fatigue, frequent urination, memory changes, weakness, yawning, stretching, seeing bright spots or flashes of light, vision loss, seeing dark spots, tingling sensations, speech problems, aphasia, tinnitus, gastric stasis, pulsating or throbbing pain on one or both sides of the head, extreme sensitivity to light (photophobia), sounds (phonophobia), or smells, worsening pain during physical activity, and vomiting, abdominal pain or heartburn, loss of appetite, lightheadedness, blurred vision, or fainting. A patient may experience one or more of the above migraine headaches, symptoms, or episodes.

The underlying migraine medications that a particular patient may be taking in accordance with the present invention are not limited. The underlying migraine medication being taken by a patient that may experience migraine breakthrough may be a GCRP antagonist or may operate by another mechanism. In an aspect, the present invention is directed to patients that are taking migraine medications that are biologics, i.e., antibodies, antibody fragments or peptides. Such biologics comprise molecules that have a mass of greater than about 900 Daltons, for example, greater than 1100 Daltons, greater than 1300 Daltons, greater than 1500 Daltons, greater than 5000 Daltons, greater than 10000 Daltons, greater than 50000 Daltons, or greater than 100000 Daltons. Examples of biologics commercially available or currently being studied for the treatment of migraine include the following. EMGALITY™ (galcanezumab-gnlm), available from Eli Lilly and Company, is a humanized IgG4 monoclonal antibody specific for calcitonin-gene related peptide (CGRP) ligand. Galcanezumab-gnlm is produced in Chinese Hamster Ovary (CHO) cells by recombinant DNA technology. Galcanezumab-gnlm is composed of two identical immunoglobulin kappa light chains and two identical immunoglobulin gamma heavy chains and has an overall molecular weight of approximately 147 kDa. AJOVY™ (fremanezumab-vfrm) injection, available from Teva Pharmaceutical Industries, is a fully humanized IgG2 Da/kappa monoclonal antibody specific for calcitonin gene-related peptide (CGRP) ligand. Fremanezumab-vfrm is produced by recombinant DNA technology in Chinese hamster ovary (CHO) cells. The antibody consists of 1324 amino acids and has a molecular weight of approximately 148 kDa. Eptinezumab, under development by Alder Biopharmaceuticals, Inc., is a fully humanized 1031 antibody manufactured using yeast (Pichia pastoris). AIMOVIG™ (erenumab-aooe) injection, available from Amgen Inc., is a human immunoglobulin G2 (IgG2) monoclonal antibody that has high affinity binding to the calcitonin gene-related peptide receptor. Erenumab-aooe is produced using recombinant DNA technology in Chinese hamster ovary (CHO) cells. It is composed of 2 heavy chains, each containing 456 amino acids, and 2 light chains of the lambda subclass, each containing 216 amino acids, with an approximate molecular weight of 150 kDa.

When a patient undergoing treatment for migraine undergoes breakthrough, e.g., has a migraine headache, treatment options may be limited particularly for patients being treated with biologics. Some patients may take common medicines, such as triptans or NSAIDS, in an attempt to obtain relief from the migraine breakthrough. However, such medicines may not be very effective.

In accordance with the present invention, patients undergoing migraine breakthrough make take a breakthrough CGRP antagonist. The CGRP antagonists for treating breakthrough migraine in accordance with the present invention are preferably non-biologic CGRP antagonists. More specifically, the non-biologic CGRP antagonists of the present invention preferably do not contain antibodies, antibody fragments or peptides. Preferably, the CGRP antagonists for use in treating breakthrough migraine in accordance with the present invention contain molecules with a mass of less than about 900 Daltons, i.e., small molecules. Examples of such non-biologic CGRP antagonists include, olcegepant, telcagepant, ubrogepant, atogepant, rimegepant, and zavegepant.

Rimegepant has the chemical formula, C₂₈H₂₈F₂N₆O₃ and the IUPAC name [(5S,6S,9R)-5-amino-6-(2,3-difluorophenyl)-6,7,8,9-tetrahydro-5H-cyclohepta[b]pyridin-9-yl] 4-(2-oxo-3H-imidazo[4,5-b]pyridin-1-yl)piperidine-1-carboxylate. Rimegepant is also known as and referred to herein as BHV-3000.

The structure of rimegepant is:

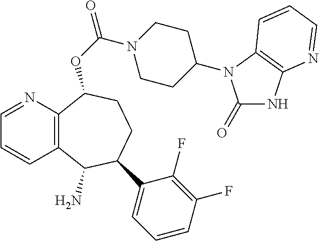 [SMILES: N[C@@H]1c2cccnc2[C@H](OC(=O)N2CCC(n3c(=O)[nH]c4ncccc43)CC2)CC[C@H]1c1cccc(F)c1F]

Rimegepant is described, for example, in WO 2011/046997 published Apr. 21, 2011.

In a preferred aspect of the invention, rimegepant is present in the form of a hemisulfate sesquihydrate salt. This preferred salt form is described in WO 2013/130402 published Sep. 6, 2013.

The chemical formula of the salt form is C₂O₂₈F₂N₆O₃.0.5H₂SO₄.1.5H₂O and the structure is as follows:

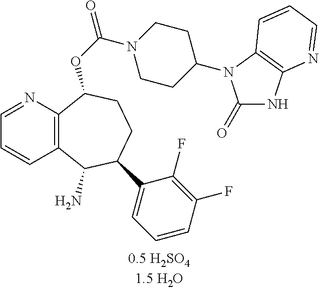 [SMILES: N[C@@H]1c2cccnc2[C@H](OC(=O)N2CCC(n3c(=O)[nH]c4ncccc43)CC2)CC[C@H]1c1cccc(F)c1F]

Another CGRP antagonist is zavegepant (also known as vazegepant), which is described in WO 2011/123232 published Oct. 6, 2011, and has the following structure (also known as BHV-3500):

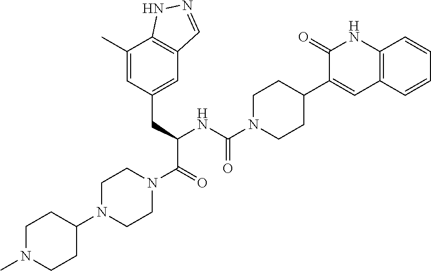 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

Another CGRP antagonist is ubrogepant, which has the following structure:

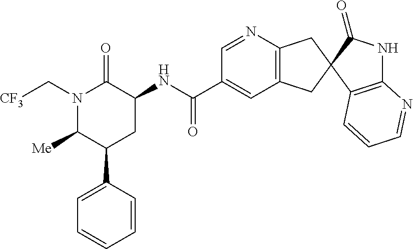 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

Another CGRP antagonist is atogepant, which has the following structure:

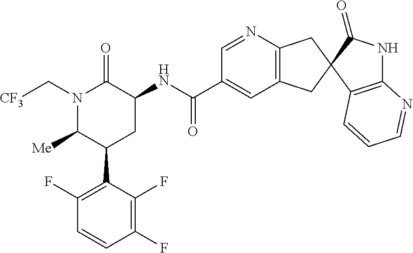 [SMILES: C[C@@H]1[C@H](c2c(F)ccc(F)c2F)C[C@H](NC(=O)c2cnc3c(c2)C[C@@]2(C3)C(=O)Nc3ncccc32)C(=O)N1CC(F)(F)F]

Another CGRP antagonist is olcegepant, which has the following structure:

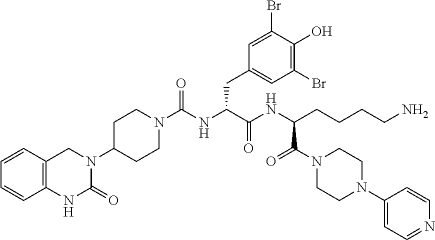 [SMILES: NCCCC[C@H](NC(=O)[C@@H](Cc1cc(Br)c(O)c(Br)c1)NC(=O)N1CCC(N2Cc3ccccc3NC2=O)CC1)C(=O)N1CCN(c2ccncc2)CC1]

Typically, in accordance with the present invention, the CGRP antagonist taken to treat migraine breakthrough is administered in the form of a pharmaceutical composition. Descriptions of the present invention are herein after described with respect to rimegepant, although such descriptions are intended to apply to other CGRP antagonists for the treatment of migraine breakthrough, for example, ubrogepant, atogepant, and zavegepant.

In an aspect of the present invention, the antibody may be galcanezumab-gnlm, and the breakthrough CGRP antagonist may be ubrogepant or atogepant. In another aspect, the antibody may be galcanezumab-gnlm, and the breakthrough CGRP antagonist may be rimegepant. In another aspect, the antibody may be galcanezumab-gnlm, and the breakthrough CGRP antagonist may be zavegepant. In another aspect, the antibody may be fremanezumab-vfrm, and the breakthrough CGRP antagonist may be ubrogepant or atogepant. In another aspect, the antibody may be fremanezumab-vfrm, and the breakthrough CGRP antagonist may be rimegepant. In another aspect, the antibody may be fremanezumab-vfrm, and the breakthrough CGRP antagonist may be zavegepant. In another aspect, the antibody may be eptinezumab, and the breakthrough CGRP antagonist may be ubrogepant or atogepant. In another aspect, the antibody may be eptinezumab, and the breakthrough CGRP antagonist may be rimegepant. In another aspect, the antibody may be eptinezumab, and the breakthrough CGRP antagonist may be zavegepant. In another aspect, the antibody may be erenumab-aooe, and the breakthrough CGRP antagonist may be ubrogepant or atogepant. In another aspect, the antibody may be erenumab-aooe, and the breakthrough CGRP antagonist may be rimegepant. In another aspect, the antibody may be erenumab-aooe, and the breakthrough CGRP antagonist may be zavegepant.

In another aspect of the present invention, the migraine medication used in the underlying treatment may include a triptan and an antibody, and the breakthrough CGRP antagonist may be ubrogepant, atogepant, rimegepant, or zavegepant. In an aspect, the triptan is selected from rizatriptan, sumatriptan, naratriptan, eletriptan, donitriptan, almotriptan, frovatriptan, avitriptan, and zolmitriptan. In another aspect, the triptan is other than rizatriptan, sumatriptan, naratriptan, eletriptan, donitriptan, almotriptan, frovatriptan, avitriptan, or zolmitriptan. In another aspect, the underlying treatment is a treatment with at least one non-triptan drug.

In another aspect, the underlying treatment takes place for one week, two weeks, three weeks, four weeks, five weeks, six weeks, seven weeks, eight weeks, nine weeks, ten weeks, eleven weeks, or twelve or more weeks. The patient may experience reduced frequency or reduced severity of migraine after treatment with the CGRP antagonist.

The pharmaceutical compositions of the present invention can be prepared in any suitable dosage form including, for example, such as tablets, capsules, powders, granules, ointments, solutions, suppositories, injections, inhalants, gels, microspheres, and aerosols.

The pharmaceutical compositions of the present invention comprising rimegepant typically also include other pharmaceutically acceptable carriers and/or excipients such as, for example, binders, lubricants, diluents, coatings, disintegrants, barrier layer components, glidants, coloring agents, solubility enhancers, gelling agents, fillers, proteins, co-factors, emulsifiers, solubilizing agents, suspending agents, flavorants, preservatives and mixtures thereof. A skilled artisan in the art would know what other pharmaceutically acceptable carriers and/or excipients could be included in the formulations according to the invention. The choice of excipients would depend on the characteristics of the compositions and on the nature of other pharmacologically active compounds in the formulation. Appropriate excipients are known to those skilled in the art (see Handbook of Pharmaceutical Excipients, fifth edition, 2005 edited by Rowe et al., McGraw Hill) and have been utilized to yield a novel sublingual formulation with unexpected properties.

Examples of pharmaceutically acceptable carriers that may be used in preparing the pharmaceutical compositions of the present invention may include, but are not limited to, fillers such as sugars, including lactose, sucrose, mannitol, or sorbitol; cellulose preparations such as maize starch, wheat starch, rice starch, potato starch, gelatin, gum tragacanth, methyl cellulose, hydroxypropyl methyl-cellulose, sodium carboxymethylcellulose, polyvinyl-pyrrolidone (PVP), talc, calcium sulphate, vegetable oils, synthetic oils, polyols, alginic acid, phosphate buffered solutions, emulsifiers, isotonic saline, pyrogen-free water and combinations thereof. If desired, disintegrating agents may be combined as well, and exemplary disintegrating agents may be, but not limited to, cross-linked polyvinyl pyrrolidone, agar, or alginic acid or a salt thereof such as sodium alginate. In an aspect of the invention, the flavoring agent is selected from mint, peppermint, berries, cherries, menthol and sodium chloride flavoring agents, and combinations thereof. In an aspect of the invention, the sweetener is selected from sugar, sucralose, aspartame, acesulfame, neotame, and combinations thereof.

In general, the pharmaceutical compositions of the present invention may be manufactured in conventional methods known in the art, for example, by means of conventional mixing, dissolving, granulating, dragee-making, levigating, emulsifying, encapsulating, entrapping, lyophilizing processes and the like.

In an aspect, the CGRP antagonist is administered at a dose of about 1-1000 mg per day. In another aspect, the CGRP antagonist is administered at a dose of about 1, 5, 10, 15, 20, 25, 30, 40, 50, 60, 70, 80, 90, 100, 200, 250, 300, 400, 500, 750, or 1000 mg per day. In an aspect, the CGRP antagonist may be administered orally. In another aspect, the CGRP antagonist may be administered intranasally.

In an aspect, the pharmaceutical composition may include about 50 mg or 100 mg of ubrogepant or a pharmaceutically acceptable salt thereof equivalent in potency to about 50 mg or 100 mg of ubrogepant. In another aspect, the pharmaceutical composition may include about 75 mg of rimegepant or a pharmaceutically acceptable salt thereof equivalent in potency to about 75 mg of rimegepant. Rimegepant may be in the form of a hemisulfate sesquihydrate salt. The pharmaceutical composition may be in the form of a tablet or a capsule.

In an aspect of the invention the pharmaceutical compositions are prepared in oral solid molded fast-dispersing dosage form, such as described in U.S. Pat. No. 9,192,580, issued Nov. 24, 2015. The phrase “fast-dispersing dosage form” refers to compositions which disintegrate or disperse within 1 to 60 seconds, preferably 1 to 30 seconds, more preferably 1 to 10 seconds and particularly 2 to 8 seconds, after being placed in contact with a fluid. The fluid is preferably that found in the oral cavity, i.e., saliva, as with oral administration.

In a preferred embodiment, the compositions of the invention are solid fast dispersing dosage forms comprising a solid network of the active ingredient, rimegepant, and a water-soluble or water-dispersible carrier containing fish gelatin. Accordingly, the carrier is inert towards the active ingredient. The network is obtained by subliming solvent from a composition in the solid state, the composition comprising the active ingredient and a solution of the carrier in the solvent. The dosage forms according to the invention can be prepared according to the process disclosed in Gregory et al., U.K. Patent No. 1,548,022 using fish gelatin as the carrier. Accordingly, an initial composition (or admixture) comprising the active ingredient and a solution of the fish gelatin carrier in a solvent is prepared followed by sublimation. The sublimation is preferably carried out by freeze drying the composition. The composition can be contained in a mold during the freeze-drying process to produce a solid form in any desired shape. The mold can be cooled using liquid nitrogen or solid carbon dioxide in a preliminary step prior to the deposition of the composition therein. After freezing the mold and composition, they are next subjected to reduced pressure and, if desired, controlled application of heat to aid in sublimation of solvent. The reduced pressure applied in the process can be below about 4 mm Hg, preferably below about 0.3 mm Hg. The freeze dried compositions can then be removed from the mold if desired or stored therein until later use.

When the process is used with active ingredients and fish gelatin as the carrier, a solid fast-dispersing dosage form is produced having the advantages associated with the use of fish gelatin described herein. Generally, fish gelatin is categorized as being from cold water and warm water fish sources and as being of the gelling or non-gelling variety. The non-gelling variety of fish gelatin, in comparison to gelling fish gelatin and bovine gelatin, contains lower proline and hydroxyproline amino acid content, which are known to be associated with cross-linking properties and gelling ability. Non-gelling fish gelatin can remain at solution concentrations of up to about 40% as well as in temperatures as low as 20° C. In an aspect of the invention, the fish gelatin used in accordance with the invention is preferably obtained from cold water fish sources and is the non-gelling type of fish gelatin. More preferably, in an aspect of the invention, the non-hydrolyzed form of non-gelling fish gelatin is used. In an alternative embodiment, spray-dried non-hydrolyzed non-gelling fish gelatin can be used. Fish gelatins suitable for use in the invention are commercially available.

The compositions according to the invention can also contain, in addition to the active ingredient arid fish gelatin carrier, other matrix forming agents and secondary components. Matrix forming agents suitable for use in the present invention include materials derived from animal or vegetable proteins, such as other gelatins, dextrins and soy, wheat and psyllium seed proteins; gums such as acacia, guar, agar, and 10 xanthan; polysaccharides; alginates; carboxymethylcelluloses; carrageenans; dextrans; pectins; synthetic polymers such as polyvinylpyrrolidone; and polypeptide/protein or polysaccharide complexes such as gelatin-acacia complexes.

Other materials which may also be incorporated into the fast-dissolving compositions of the present invention include sugars such as mannitol, dextrose, lactose, galactose, and trehalose; cyclic sugars such as cyclodextrin; inorganic salts such as sodium phosphate, sodium chloride and aluminum silicates; and amino acids having from 2 to 12 carbon atoms such as glycine, L-alanine, L-aspartic acid, L-glutamic acid, L-hydroxyproline, L-isoleucine, L-leucine and L-phenylalanine. One or more matrix forming agents may be incorporated into the solution or suspension prior to solidification (freezing). The matrix forming agent may be present in addition to a surfactant or to the exclusion of a surfactant. In addition to forming the matrix, the matrix forming agent may aid in maintaining the dispersion of any active ingredient within the solution of suspension. This is especially helpful in the case of active agents that are not sufficiently soluble in water and must, therefore, be suspended rather than dissolved. Secondary components such as preservatives, antioxidants, surfactants, viscosity enhancers, coloring agents, flavoring agents, pH modifiers, sweeteners or taste-masking agents may also be incorporated into the fast-dissolving compositions. Suitable coloring agents include red, black and yellow iron oxides and FD & C dyes such as FD&C Blue No. 2 and FD&C Red No. 40 available from Ellis & Everard. Suitable flavoring agents include mint, raspberry, licorice, orange, lemon, grapefruit, caramel, vanilla, cherry and grape flavors and combinations of these. Suitable pH modifiers include the edible acids and bases, such as citric acid, tartaric acid, phosphoric acid, hydrochloric acid, maleic acid and sodium hydroxide. Suitable sweeteners include, for example, sucralose, aspartame, acesulfame K and thaumatin. Suitable taste-masking agents include, for example, sodium bicarbonate, ion exchange resins, cyclodextrin inclusion compounds, adsorbates or microencapsulated actives.

Typical routes of administering the pharmaceutical compositions of the invention include, without limitation, oral, topical, transdermal, inhalation, parenteral, sublingual, buccal, rectal, vaginal, and intranasal. The term “parenteral” as used herein includes subcutaneous injections, intravenous, intramuscular, intrasternal injection or infusion techniques. Pharmaceutical compositions according to certain embodiments of the present invention are formulated so as to allow the active ingredients contained therein to be bioavailable upon administration of the composition to a patient. Compositions that will be administered to a subject or patient may take the form of one or more dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000).

Solid compositions are normally formulated in dosage units providing from about 1 to about 1000 mg of the active ingredient per dose. Some examples of solid dosage units are 0.1 mg, 1 mg, 10 mg, 37.5 mg, 75 mg, 100 mg, 150 mg, 300 mg, 500 mg, 600 mg and 1000 mg. Typical dose ranges in accordance with the present invention include from about 10-600 mg, 25-300 mg, 25-150 mg, 50-100 mg, 60-90 mg, and 70-80 mg. Liquid compositions are generally in a unit dosage range of 1-100 mg/mL. Some examples of liquid dosage units are 0.1 mg/mL, 1 mg/mL, 10 mg/mL, 25 mg/mL, 50 mg/mL, and 100 mg/mL.

In an aspect, the pharmaceutical composition may include about 50-60 weight % rimegepant hemisulfate sesquihydrate, about 30-35 weight % microcrystalline cellulose, about 2-7 weight % hydroxypropyl cellulose, about 3-7 weight % croscarmellose sodium, and about 0.1-1.0 weight % magnesium stearate. In another aspect, the pharmaceutical composition may include about 57.1 weight % rimegepant hemisulfate sesquihydrate, about 33.4 weight % microcrystalline cellulose, about 4.0 weight % hydroxypropyl cellulose, about 5.0 weight % croscarmellose sodium, and about 0.5 weight % magnesium stearate. In another aspect, the pharmaceutical composition may include from about 70-80 weight % rimegepant hemisulfate sesquihydrate, about 10-20 weight % fish gelatin, about 10-20 weight % of a filler, and 0.1-5.0 weight % of a flavorant.

In another aspect, the filler may be mannitol.

In another aspect, the pharmaceutical composition may be a spayed-dried composition.

In another aspect, the sprayed-dried composition may include hypermellose succinate acetate and a therapeutically effective amount of a breakthrough CGRP antagonist, or a pharmaceutically acceptable salt thereof. In an aspect the sprayed-dried composition may include ubrogepant. A matrix containing ubrogepant may be prepared by spray-drying a solution of ubrogepant and hypromellose acetate succinate type LG® (Shin Etsu, article of commerce), blending the dried granulate with various excipients and pressing the mixture into tablets, as described in U.S. Patent Publication No. 2018/0092899 published Apr. 5, 2018.

In some embodiments, a method may include administering to a subject one or more additional agent(s) simultaneously or sequentially with the rimegepant. In some embodiments, an additional agent may be an anti-headache medication such as an example anti-headache medication (e.g., 5-HT1 agonists, triptans, ergot alkaloids, opiates, adrenergic antagonists, NSAIDs or antibodies) known in the art. In some embodiments, a therapeutic effect may be greater as compared to use of rimegepant or one or more additional agent(s) alone. Accordingly, a synergistic effect between rimegepant and the one or more additional agents may be achieved. In some embodiments, the one or more additional agent(s) may be taken by a subject prophylactically.

In an aspect, the invention also provides kits for use in the instant methods. Kits can include one or more containers comprising a pharmaceutical composition described herein and instructions for use in accordance with any of the methods described herein. Generally, these instructions comprise a description of administration of the pharmaceutical composition to treat, ameliorate or prevent headache (such as migraine), or other CRGP disorder, according to any of the methods described herein. The kit may, for example, comprise a description of selecting an individual suitable for treatment based on identifying whether that individual has headache or whether the individual is at risk of having headache. The instructions are typically provided in the form of a package insert, or label, in accordance with the requirements of the regulatory having authority over the jurisdiction where the pharmaceutical composition is to be provided to patients.

### EXAMPLES

The following examples illustrate the invention and are not intended to limit the scope of the invention.

### Example 1

Tablet Manufacture—A batch is prepared to manufacture tablets containing a dose of 75 mg of rimegepant as follows. The composition of the batch is set forth below in Table 1. Tablets are made from the batch as indicated.

TABLE 1

| | | | |
|---|---|---|---|
| | | | Amount per |
| | Percent | Amount | 100,000 |
| | per | per | Tablet |
| Ingredient | Tablet | Tablet (mg) | Batch (g) |

Intra-granular

| | | | |
|---|---|---|---|
| Rimegepant (as hemisulfate | 57.11 | 85.67 | 8575.5 |
| sesquihydrate equivalent to 75 | | | |
| mg as base) | | | |
| Microcrystalline cellulose, NF | 13.39 | 20.09 | 2,011.0 |
| Hydroxypropyl Cellulose), | 4.00 | 6.00 | 600.6 |
| USP/NF (Klucel EXF PHARM) | | | |
| Croscarmellose Sodium NF | 2.50 | 3.75 | 375.4 |
| Purified Water USP | q.s. | N/A | 0¹ |
| Intragranular Dispensed Solids | | | 11562 |

Extra-granular

| | | | |
|---|---|---|---|
| Microcrystalline cellulose NF | 20.00 | 30.00 | 3,003.0 |
| Croscarmellose Sodium NF | 2.50 | 3.75 | 375.4 |
| Magnesium Stearate NF | 0.50 | 0.75 | 75.08 |
| Total Core Tablet | 100.0 | 150 | 15015 |

¹Purified Water is removed in-process. An excess amount is dispensed. The portion consumed is documented. Intragranular Dispensed Solids does not include water.

The rimegepant hemisulfate sesquihydrate and all excipients are weighed.

Pass the rimegepant hemisulfate sesquihydrate, microcrystalline cellulose (intragranular portion), hydroxypropyl cellulose, and croscarmellose sodium (intragranular portion) through a 20-mesh screen.

Load the sieved mixture from 2 into a suitable granulator equipped with an appropriate size bowl & dry mix for 10 minutes. Set impeller speed to low & turn chopper off.

While mixing, equip the granulator with a spray tip and add purified water until endpoint is reached.

Mix wet mass for 30 seconds with impeller set to low and chopper set to low.

Discharge the wet mass into expansion chamber of fluid bed dryer. Dry to target LOD of <2%.

Mill the dried granules using the Comil with an appropriate screen (0.075R) and spacer (0.050). Perform bulk and tapped density & particle size distribution analyses. Record results. Calculate Carr Index & Carr Index mean from two samples.

Calculate the fractional yield. Recalculate the extragranular quantities.

Pass the microcrystalline cellulose and croscarmellose through a 20-mesh screen.

Combine the milled granulation with the recalculated microcrystalline cellulose (extragranular portion), croscarmellose sodium (extragranular portion) in a 2-cubic foot tote & blend for 150 revolutions.

Pass the magnesium stearate through 30-mesh screen.

Add screened magnesium stearate to the 2 cubic foot tote contents & blend for 75 revolutions. Collect blend uniformity samples per plan.

Perform bulk & tapped density and particle size analyses & calculate Carr Index.

Discharge into a suitable container and weigh.

Set up 716-station rotary tablet press with 7 mm round concave plain tooling. Adjust number of stations as needed.

Adjust the press to achieve the following specifications for the tablets: Friability of ≤0.3% loss; Hardness of 10-14 kP; Thickness of 3.60-4.10 mm; and Disintegration of ≥2:30 minutes.

Conduct in-process testing as follows:

Tablet friability and disintegration at beginning, middle and end of run

Tablet hardness, tablet thickness, individual tablet weights, average tablet weights, and appearance at 15 minute intervals

Pass the tablets through a de-duster and metal detector.

Package tablets in double PE bags in a suitable container.

### Example 2

Clinical Trial—BHV3000-201: Open Label Safety Study in Acute Treatment of Migraine (ClinicalTrials.gov Identifier: NCT 03266588)

A phase 2/3 clinical study is conducted with about 2000 participants, as follows.

Study Description

Brief Summary:

The purpose of this study is to evaluate safety and tolerability of BHV-3000 (rimegepant).

| Condition or disease | Intervention/treatment | Phase |
|---|---|---|
| Migraine | Drug: Rimegepant | Phase 2/Phase 3 |

Study Design
Study Type: Interventional (Clinical Trial)
Estimated Enrollment: 2000 participants
Intervention Model: Single Group Assignment
Masking: None (Open Label)
Primary Purpose: Treatment
Official Title: A Multicenter, Open Label Long-Term Safety Study of BHV-3000 in the Acute Treatment of Migraine
Actual Study Start Date: Aug. 30, 2017
Estimated Primary Completion Date: July 2019
Estimated Study Completion Date: July 2019

Arms and Interventions

| Arm | Intervention/treatment |
|---|---|
| Experimental: Rimegepant | Drug: Rimegepant |
| | 75 mg oral tablet |
| | Other Name: BHV3000 |

Outcome Measures

Primary Outcome Measures:

To assess the safety and tolerability of rimegepant (BHV-3000) by measuring the frequency and severity of adverse events and discontinuations due to adverse events [Time Frame: 52 weeks] Number of subjects with treatment-emergent adverse events as assessed through laboratory tests, ECGs, physical exam findings (safety and tolerability)

Secondary Outcome Measures: ALT or AST >3×ULN with total bilirubin >2×ULN [Time Frame: 52 weeks] elevated liver function tests

Hepatic related adverse events and hepatic related adverse events that lead to discontinuation [Time Frame: 52 weeks] adverse events related to liver

Further details concerning the clinical study including eligibility criteria, contacts and locations and more information can be found at www.clinicaltrials.gov for ClinicalTrials.gov Identifier: NCT03266588.

### Example 3

Results from the clinical trial described in Example 2 are summarized as follows. Study BHV-3000-201 demonstrated initial positive results. The interim analysis (database cutoff of Nov. 21, 2018) demonstrated that the safety and tolerability of long-term dosing of rimegepant in patients with migraine is consistent with the profile observed in phase 1-3 studies to date. Patients were allowed to treat migraine attacks of all severities (mild to severe) up to once daily for a full year. The initial results for hepatic safety and tolerability of rimegepant 75 mg in study participants is based upon review of both adverse events and regularly scheduled liver function tests. Interim hepatic data were reviewed by an external and independent panel of liver experts. There were no liver cases assessed as probably related to study drug and there were no Hy's Law cases identified. The panel concluded that there was no liver safety signal detected to date, including a subset of patients with near-daily dosing (≥15 doses/month). In the aggregate, it was noted that compared to migraine trials with drugs other than rimegepant, there was a very low incidence of overall elevations of liver function test abnormalities in rimegepant treated patients (1.0% incidence of serum ALT or AST >3×ULN). Subjects will continue to participate in Study 201 with additional data analyses to be submitted in the NDA and required 120-day safety updates.

Quite surprisingly, in addition to the interim safety analysis, preliminary open-label data on headache frequency from Study 201 shows that intermittent dosing of rimegepant 75 mg is associated with a reduction in migraine days per month, i.e., 30 days, suggesting a preventive effect of rimegepant. In an exploratory analysis in Study 201, patients who experienced 15 or more migraine days/month during the standard of care observation period, i.e., before treatment started with rimegepant, demonstrated a 4 day mean reduction in headaches/month by 12 weeks of intermittent dosing with rimegepant 75 mg. Approximately 40% of patients who had 15 or more headache days/month during the observation period showed at least a 30% or more reduction in their monthly number of headache days by 12 weeks of treatment with rimegepant. Reductions in the mean number of headache days per month was observed beginning as early as the first month and continued in subsequent months of therapy.

### Example 4

Breakthrough Treatment of Patients

Certain subjects in the study described in Example 2 were undergoing treatment with a biologic CGRP inhibitor, erenumab-aooe. Records of migraine headache breakthrough are shown in Tables 2-6 below. Subjects that experienced breakthrough, administered rimegepant. Quite surprisingly, subjects that administered rimegepant obtained relief from the migraine headache without the need to take other medications such as triptans or NSAIDS.

TABLE 2

| | | | | Take other | Take study | |
|---|---|---|---|---|---|---|
| | | | | medication | medication | |
| | | Have | | [Observation | [Treatment | Number of |
| Subject ID | Report Datetime | migraine | Severity | phase] | phase] | tablets |
| 1196 | 4 Dec. 2017 | Yes | Moderate | Yes | — | — |
| | 19:12:00 | | | | | |
| 1196 | 5 Dec. 2017 | Yes | Moderate | Yes | — | — |
| | 21:14:00 | | | | | |
| 1196 | 6 Dec. 2017 | Yes | Moderate | Yes | — | — |
| | 21:16:00 | | | | | |
| 1196 | 7 Dec. 2017 | Yes | Moderate | Yes | — | — |
| | 20:31:00 | | | | | |
| 1196 | 8 Dec. 2017 | Yes | Moderate | Yes | — | — |
| | 21:19:00 | | | | | |
| 1196 | 9 Dec. 2017 | No | — | — | — | — |
| | 21:15:00 | | | | | |
| 1196 | 10 Dec. 2017 | No | — | — | — | — |
| | 22:10:00 | | | | | |
| 1196 | 11 Dec. 2017 | No | — | — | — | — |
| | 21:08:00 | | | | | |
| 1196 | 13 Dec. 2017 | No | — | — | — | — |
| | 20:56:00 | | | | | |
| 1196 | 14 Dec. 2017 | Yes | Moderate | Yes | — | — |
| | 23:06:00 | | | | | |
| 1196 | 16 Dec. 2017 | No | — | — | — | — |
| | 21:18:00 | | | | | |
| 1196 | 17 Dec. 2017 | No | — | — | — | — |
| | 21:04:00 | | | | | |
| 1196 | 18 Dec. 2017 | No | — | — | — | — |
| | 23:50:00 | | | | | |
| 1196 | 19 Dec. 2017 | No | — | — | — | — |
| | 22:01:00 | | | | | |
| 1196 | 20 Dec. 2017 | No | — | — | — | — |
| | 21:02:00 | | | | | |
| 1196 | 21 Dec. 2017 | Yes | Mild | Yes | — | — |
| | 22:49:00 | | | | | |
| 1196 | 22 Dec. 2017 | Yes | Moderate | Yes | — | — |
| | 21:00:00 | | | | | |
| 1196 | 23 Dec. 2017 | Yes | Moderate | Yes | — | — |
| | 22:53:00 | | | | | |
| 1196 | 24 Dec. 2017 | No | — | — | — | — |
| | 22:40:00 | | | | | |
| 1196 | 25 Dec. 2017 | No | — | — | — | — |
| | 21:02:00 | | | | | |
| 1196 | 26 Dec. 2017 | No | — | — | — | — |
| | 21:19:00 | | | | | |
| 1196 | 27 Dec. 2017 | No | — | — | — | — |
| | 21:10:00 | | | | | |
| 1196 | 28 Dec. 2017 | Yes | Moderate | Yes | — | — |
| | 22:21:00 | | | | | |
| 1196 | 29 Dec. 2017 | No | — | — | — | — |
| | 21:00:00 | | | | | |
| 1196 | 30 Dec. 2017 | Yes | Moderate | Yes | — | — |
| | 21:01:00 | | | | | |
| 1196 | 31 Dec. 2017 | Yes | Mild | Yes | — | — |
| | 22:12:00 | | | | | |
| 1196 | 1 Jan. 2018 | No | — | — | — | — |
| | 21:01:00 | | | | | |
| 1196 | 2 Jan. 2018 | No | — | — | — | — |
| | 21:49:00 | | | | | |
| 1196 | 3 Jan. 2018 | No | — | — | — | — |
| | 21:12:00 | | | | | |
| 1196 | 4 Jan. 2018 | No | — | — | — | — |
| | 21:30:00 | | | | | |
| 1196 | 5 Jan. 2018 | Yes | Moderate | — | No | — |
| | 20:49:00 | | | | | |
| 1196 | 6 Jan. 2018 | No | — | — | No | — |
| | 21:00:00 | | | | | |
| 1196 | 7 Jan. 2018 | No | — | — | No | — |
| | 21:03:00 | | | | | |
| 1196 | 8 Jan. 2018 | No | — | — | No | — |
| | 21:09:00 | | | | | |
| 1196 | 9 Jan. 2018 | No | — | — | No | — |
| | 23:02:00 | | | | | |
| 1196 | 10 Jan. 2018 | No | — | — | No | — |
| | 21:00:00 | | | | | |
| 1196 | 11 Jan. 2018 | No | — | — | No | — |
| | 21:41:00 | | | | | |
| 1196 | 12 Jan. 2018 | Yes | Mild | — | Yes | 1 |
| | 21:01:00 | | | | | |
| 1196 | 14 Jan. 2018 | No | — | — | No | — |
| | 21:33:00 | | | | | |
| 1196 | 15 Jan. 2018 | Yes | Moderate | — | Yes | 1 |
| | 21:14:00 | | | | | |
| 1196 | 16 Jan. 2018 | No | — | — | No | — |
| | 21:50:00 | | | | | |
| 1196 | 17 Jan. 2018 | No | — | — | No | — |
| | 20:42:00 | | | | | |
| 1196 | 18 Jan. 2018 | No | — | — | No | — |
| | 21:10:00 | | | | | |
| 1196 | 19 Jan. 2018 | Yes | Moderate | — | Yes | 1 |
| | 21:37:00 | | | | | |
| 1196 | 20 Jan. 2018 | No | — | — | No | — |
| | 22:05:00 | | | | | |
| 1196 | 21 Jan. 2018 | No | — | — | No | — |
| | 21:04:00 | | | | | |
| 1196 | 22 Jan. 2018 | Yes | Mild | — | Yes | 1 |
| | 21:18:00 | | | | | |
| 1196 | 23 Jan. 2018 | No | — | — | No | — |
| | 21:36:00 | | | | | |
| 1196 | 24 Jan. 2018 | Yes | Mild | — | Yes | 1 |
| | 20:25:00 | | | | | |
| 1196 | 25 Jan. 2018 | No | — | — | No | — |
| | 21:31:00 | | | | | |
| 1196 | 26 Jan. 2018 | No | — | — | No | — |
| | 20:57:00 | | | | | |
| 1196 | 27 Jan. 2018 | No | — | — | No | — |
| | 21:05:00 | | | | | |
| 1196 | 28 Jan. 2018 | No | — | — | No | — |
| | 21:09:00 | | | | | |
| 1196 | 29 Jan. 2018 | Yes | Moderate | — | Yes | 1 |
| | 21:09:00 | | | | | |
| 1196 | 30 Jan. 2018 | No | — | — | No | — |
| | 21:44:00 | | | | | |
| 1196 | 31 Jan. 2018 | No | — | — | No | — |
| | 21:06:00 | | | | | |
| 1196 | 1 Feb. 2018 | No | — | — | No | — |
| | 22:25:00 | | | | | |
| 1196 | 2 Feb. 2018 | No | — | — | No | — |
| | 21:01:00 | | | | | |
| 1196 | 3 Feb. 2018 | Yes | Moderate | — | Yes | 1 |
| | 21:09:00 | | | | | |
| 1196 | 4 Feb. 2018 | No | — | — | No | — |
| | 21:23:00 | | | | | |
| 1196 | 5 Feb. 2018 | No | — | — | No | — |
| | 21:02:00 | | | | | |
| 1196 | 6 Feb. 2018 | No | — | — | No | — |
| | 21:01:00 | | | | | |
| 1196 | 7 Feb. 2018 | Yes | Moderate | — | Yes | 1 |
| | 20:07:00 | | | | | |
| 1196 | 8 Feb. 2018 | No | — | — | No | — |
| | 21:20:00 | | | | | |
| 1196 | 9 Feb. 2018 | Yes | Moderate | — | Yes | 1 |
| | 23:18:00 | | | | | |
| 1196 | 10 Feb. 2018 | No | — | — | No | — |
| | 22:24:00 | | | | | |
| 1196 | 11 Feb. 2018 | No | — | — | No | — |
| | 21:13:00 | | | | | |
| 1196 | 12 Feb. 2018 | Yes | Moderate | — | Yes | 1 |
| | 21:11:00 | | | | | |
| 1196 | 13 Feb. 2018 | No | — | — | No | — |
| | 23:07:00 | | | | | |
| 1196 | 14 Feb. 2018 | Yes | Mild | — | No | — |
| | 21:15:00 | | | | | |
| 1196 | 15 Feb. 2018 | Yes | Moderate | — | Yes | 1 |
| | 20:40:00 | | | | | |
| 1196 | 16 Feb. 2018 | No | — | — | No | — |
| | 20:59:00 | | | | | |
| 1196 | 17 Feb. 2018 | No | — | — | No | — |
| | 21:00:00 | | | | | |
| 1196 | 18 Feb. 2018 | No | — | — | No | — |
| | 20:42:00 | | | | | |
| 1196 | 19 Feb. 2018 | No | — | — | No | — |
| | 21:03:00 | | | | | |
| 1196 | 20 Feb. 2018 | Yes | Mild | — | Yes | 1 |
| | 21:08:00 | | | | | |
| 1196 | 21 Feb. 2018 | No | — | — | No | — |
| | 21:16:00 | | | | | |
| 1196 | 22 Feb. 2018 | Yes | Moderate | — | Yes | 1 |
| | 21:05:00 | | | | | |
| 1196 | 23 Feb. 2018 | No | — | — | No | — |
| | 19:49:00 | | | | | |
| 1196 | 24 Feb. 2018 | No | — | — | No | — |
| | 21:30:00 | | | | | |
| 1196 | 25 Feb. 2018 | Yes | Moderate | — | Yes | 1 |
| | 21:01:00 | | | | | |
| 1196 | 26 Feb. 2018 | No | — | — | No | — |
| | 22:21:00 | | | | | |
| 1196 | 27 Feb. 2018 | No | — | — | No | — |
| | 21:45:00 | | | | | |
| 1196 | 28 Feb. 2018 | Yes | Mild | — | Yes | 1 |
| | 20:53:00 | | | | | |
| 1196 | 1 Mar. 2018 | No | — | — | No | — |
| | 20:34:00 | | | | | |
| 1196 | 2 Mar. 2018 | No | — | — | No | — |
| | 21:05:00 | | | | | |
| 1196 | 3 Mar. 2018 | No | — | — | No | — |
| | 20:58:00 | | | | | |
| 1196 | 4 Mar. 2018 | No | — | — | No | — |
| | 21:24:00 | | | | | |
| 1196 | 5 Mar. 2018 | No | — | — | No | — |
| | 20:53:00 | | | | | |
| 1196 | 6 Mar. 2018 | Yes | Moderate | — | Yes | 1 |
| | 20:51:00 | | | | | |
| 1196 | 7 Mar. 2018 | No | — | — | No | — |
| | 21:02:00 | | | | | |
| 1196 | 8 Mar. 2018 | No | — | — | No | — |
| | 21:12:00 | | | | | |
| 1196 | 9 Mar. 2018 | No | — | — | No | — |
| | 21:15:00 | | | | | |
| 1196 | 10 Mar. 2018 | No | — | — | No | — |
| | 21:39:00 | | | | | |
| 1196 | 11 Mar. 2018 | No | — | — | No | — |
| | 21:14:00 | | | | | |
| 1196 | 12 Mar. 2018 | No | — | — | No | — |
| | 21:12:00 | | | | | |
| 1196 | 13 Mar. 2018 | Yes | Moderate | — | Yes | 1 |
| | 21:17:00 | | | | | |
| 1196 | 14 Mar. 2018 | No | — | — | No | — |
| | 21:08:00 | | | | | |
| 1196 | 15 Mar. 2018 | No | — | — | No | — |
| | 21:16:00 | | | | | |
| 1196 | 17 Mar. 2018 | Yes | Moderate | — | Yes | 1 |
| | 21:00:00 | | | | | |
| 1196 | 18 Mar. 2018 | No | — | — | No | — |
| | 20:51:00 | | | | | |
| 1196 | 19 Mar. 2018 | No | — | — | No | — |
| | 20:32:00 | | | | | |
| 1196 | 20 Mar. 2018 | No | — | — | No | — |
| | 21:19:00 | | | | | |
| 1196 | 21 Mar. 2018 | Yes | Moderate | — | Yes | 1 |
| | 20:32:00 | | | | | |
| 1196 | 22 Mar. 2018 | No | — | — | No | — |
| | 20:35:00 | | | | | |
| 1196 | 23 Mar. 2018 | Yes | Moderate | — | Yes | 1 |
| | 21:14:00 | | | | | |
| 1196 | 24 Mar. 2018 | No | — | — | No | — |
| | 20:52:00 | | | | | |
| 1196 | 25 Mar. 2018 | No | — | — | No | — |
| | 22:31:00 | | | | | |
| 1196 | 26 Mar. 2018 | No | — | — | No | — |
| | 21:10:00 | | | | | |
| 1196 | 27 Mar. 2018 | Yes | Moderate | — | Yes | 1 |
| | 22:11:00 | | | | | |
| 1196 | 28 Mar. 2018 | No | — | — | No | — |
| | 21:02:00 | | | | | |
| 1196 | 29 Mar. 2018 | Yes | Moderate | — | Yes | 1 |
| | 20:24:00 | | | | | |
| 1196 | 31 Mar. 2018 | No | — | — | No | — |
| | 22:00:00 | | | | | |
| 1196 | 1 Apr. 2018 | Yes | Moderate | — | Yes | 1 |
| | 20:23:00 | | | | | |
| 1196 | 2 Apr. 2018 | No | — | — | No | — |
| | 20:48:00 | | | | | |
| 1196 | 3 Apr. 2018 | No | — | — | No | — |
| | 21:04:00 | | | | | |
| 1196 | 4 Apr. 2018 | Yes | Severe | — | Yes | 1 |
| | 21:03:00 | | | | | |
| 1196 | 6 Apr. 2018 | Yes | Moderate | — | Yes | 1 |
| | 20:49:00 | | | | | |
| 1196 | 7 Apr. 2018 | No | — | — | No | — |
| | 21:34:00 | | | | | |
| 1196 | 8 Apr. 2018 | No | — | — | No | — |
| | 20:54:00 | | | | | |
| 1196 | 9 Apr. 2018 | No | — | — | No | — |
| | 21:00:00 | | | | | |
| 1196 | 10 Apr. 2018 | No | — | — | No | — |
| | 21:00:00 | | | | | |
| 1196 | 11 Apr. 2018 | Yes | Moderate | — | Yes | 1 |
| | 20:49:00 | | | | | |
| 1196 | 12 Apr. 2018 | No | — | — | No | — |
| | 21:31:00 | | | | | |
| 1196 | 13 Apr. 2018 | Yes | Severe | — | Yes | 1 |
| | 21:14:00 | | | | | |
| 1196 | 14 Apr. 2018 | No | — | — | No | — |
| | 21:02:00 | | | | | |
| 1196 | 15 Apr. 2018 | No | — | — | No | — |
| | 21:02:00 | | | | | |
| 1196 | 16 Apr. 2018 | Yes | Moderate | — | Yes | 1 |
| | 19:52:00 | | | | | |
| 1196 | 17 Apr. 2018 | Yes | Moderate | — | Yes | 1 |
| | 21:06:00 | | | | | |
| 1196 | 18 Apr. 2018 | No | — | — | No | — |
| | 21:34:00 | | | | | |
| 1196 | 19 Apr. 2018 | Yes | Moderate | — | Yes | 1 |
| | 21:00:00 | | | | | |
| 1196 | 20 Apr. 2018 | No | — | — | No | — |
| | 23:02:00 | | | | | |
| 1196 | 21 Apr. 2018 | Yes | Severe | — | Yes | 1 |
| | 21:03:00 | | | | | |
| 1196 | 22 Apr. 2018 | No | — | — | No | — |
| | 21:45:00 | | | | | |
| 1196 | 23 Apr. 2018 | No | — | — | No | — |
| | 21:01:00 | | | | | |
| 1196 | 24 Apr. 2018 | No | — | — | No | — |
| | 19:16:00 | | | | | |
| 1196 | 25 Apr. 2018 | Yes | Moderate | — | Yes | 1 |
| | 21:05:00 | | | | | |
| 1196 | 26 Apr. 2018 | No | — | — | No | — |
| | 21:04:00 | | | | | |
| 1196 | 27 Apr. 2018 | No | — | — | No | — |
| | 22:09:00 | | | | | |
| 1196 | 29 Apr. 2018 | Yes | Moderate | — | Yes | 1 |
| | 20:12:00 | | | | | |
| 1196 | 30 Apr. 2018 | No | — | — | No | — |
| | 21:17:00 | | | | | |
| 1196 | 1 May 2018 | No | — | — | No | — |
| | 21:03:00 | | | | | |
| 1196 | 2 May 2018 | No | — | — | No | — |
| | 21:00:00 | | | | | |
| 1196 | 3 May 2018 | Yes | Mild | — | No | — |
| | 21:15:00 | | | | | |
| 1196 | 4 May 2018 | Yes | Moderate | — | Yes | 1 |
| | 21:10:00 | | | | | |
| 1196 | 5 May 2018 | Yes | Moderate | — | Yes | 1 |
| | 21:17:00 | | | | | |
| 1196 | 6 May 2018 | No | — | — | No | — |
| | 21:03:00 | | | | | |
| 1196 | 7 May 2018 | No | — | — | No | — |
| | 21:13:00 | | | | | |
| 1196 | 8 May 2018 | No | — | — | No | — |
| | 21:05:00 | | | | | |
| 1196 | 9 May 2018 | No | — | — | No | — |
| | 20:36:00 | | | | | |
| 1196 | 10 May 2018 | No | — | — | No | — |
| | 20:48:00 | | | | | |
| 1196 | 11 May 2018 | No | — | — | No | — |
| | 22:44:00 | | | | | |
| 1196 | 12 May 2018 | Yes | Moderate | — | Yes | 1 |
| | 22:25:00 | | | | | |
| 1196 | 13 May 2018 | No | — | — | No | — |
| | 21:04:00 | | | | | |
| 1196 | 14 May 2018 | Yes | Moderate | — | Yes | 1 |
| | 20:17:00 | | | | | |
| 1196 | 15 May 2018 | No | — | — | No | — |
| | 21:15:00 | | | | | |
| 1196 | 16 May 2018 | No | — | — | No | — |
| | 20:45:00 | | | | | |
| 1196 | 17 May 2018 | Yes | Moderate | — | Yes | 1 |
| | 21:01:00 | | | | | |
| 1196 | 18 May 2018 | No | — | — | No | — |
| | 22:38:00 | | | | | |
| 1196 | 19 May 2018 | No | — | — | No | — |
| | 22:02:00 | | | | | |
| 1196 | 20 May 2018 | Yes | Moderate | — | Yes | 1 |
| | 21:21:00 | | | | | |
| 1196 | 21 May 2018 | No | — | — | No | — |
| | 21:01:00 | | | | | |
| 1196 | 22 May 2018 | Yes | Mild | — | Yes | 1 |
| | 20:53:00 | | | | | |
| 1196 | 23 May 2018 | No | — | — | No | — |
| | 21:24:00 | | | | | |
| 1196 | 24 May 2018 | Yes | Moderate | — | Yes | 1 |
| | 21:10:00 | | | | | |
| 1196 | 25 May 2018 | No | — | — | No | — |
| | 21:00:00 | | | | | |
| 1196 | 26 May 2018 | No | — | — | No | — |
| | 22:06:00 | | | | | |
| 1196 | 27 May 2018 | No | — | — | No | — |
| | 22:14:00 | | | | | |
| 1196 | 28 May 2018 | Yes | Moderate | — | Yes | 1 |
| | 21:03:00 | | | | | |
| 1196 | 29 May 2018 | Yes | Moderate | — | Yes | 1 |
| | 20:30:00 | | | | | |
| 1196 | 30 May 2018 | No | — | — | No | — |
| | 20:37:00 | | | | | |
| 1196 | 31 May 2018 | Yes | Moderate | — | Yes | 1 |
| | 21:49:00 | | | | | |
| 1196 | 1 Jun. 2018 | No | — | — | No | — |
| | 22:00:00 | | | | | |
| 1196 | 3 Jun. 2018 | No | — | — | No | — |
| | 21:04:00 | | | | | |
| 1196 | 4 Jun. 2018 | No | — | — | No | — |
| | 21:08:00 | | | | | |
| 1196 | 5 Jun. 2018 | No | — | — | No | — |
| | 21:04:00 | | | | | |
| 1196 | 6 Jun. 2018 | Yes | Moderate | — | Yes | 1 |
| | 21:15:00 | | | | | |
| 1196 | 8 Jun. 2018 | No | — | — | No | — |
| | 20:59:00 | | | | | |
| 1196 | 9 Jun. 2018 | No | — | — | No | — |
| | 21:20:00 | | | | | |
| 1196 | 10 Jun. 2018 | No | — | — | No | — |
| | 22:57:00 | | | | | |
| 1196 | 11 Jun. 2018 | No | — | — | No | — |
| | 21:04:00 | | | | | |
| 1196 | 12 Jun. 2018 | Yes | Moderate | — | Yes | 1 |
| | 21:04:00 | | | | | |
| 1196 | 14 Jun. 2018 | No | — | — | No | — |
| | 21:15:00 | | | | | |
| 1196 | 16 Jun. 2018 | Yes | Moderate | — | Yes | 1 |
| | 22:06:00 | | | | | |
| 1196 | 17 Jun. 2018 | No | — | — | No | — |
| | 21:17:00 | | | | | |
| 1196 | 18 Jun. 2018 | Yes | Moderate | — | Yes | 1 |
| | 11:47:00 | | | | | |
| 1196 | 18 Jun. 2018 | No | — | — | No | — |
| | 21:12:00 | | | | | |
| 1196 | 19 Jun. 2018 | Yes | Moderate | — | Yes | 1 |
| | 20:31:00 | | | | | |
| 1196 | 20 Jun. 2018 | No | — | — | No | — |
| | 22:09:00 | | | | | |
| 1196 | 21 Jun. 2018 | No | — | — | No | — |
| | 22:11:00 | | | | | |
| 1196 | 22 Jun. 2018 | No | — | — | No | — |
| | 21:33:00 | | | | | |
| 1196 | 23 Jun. 2018 | No | — | — | No | — |
| | 21:45:00 | | | | | |
| 1196 | 24 Jun. 2018 | No | — | — | No | — |
| | 21:46:00 | | | | | |
| 1196 | 25 Jun. 2018 | Yes | Moderate | — | Yes | 1 |
| | 21:03:00 | | | | | |
| 1196 | 26 Jun. 2018 | No | — | — | No | — |
| | 21:20:00 | | | | | |
| 1196 | 27 Jun. 2018 | No | — | — | No | — |
| | 21:50:00 | | | | | |
| 1196 | 28 Jun. 2018 | Yes | Moderate | — | Yes | 1 |
| | 21:04:00 | | | | | |
| 1196 | 29 Jun. 2018 | No | — | — | No | — |
| | 21:19:00 | | | | | |
| 1196 | 30 Jun. 2018 | No | — | — | No | — |
| | 22:03:00 | | | | | |
| 1196 | 1 Jul. 2018 | Yes | Mild | — | Yes | 1 |
| | 21:21:00 | | | | | |
| 1196 | 2 Jul. 2018 | No | — | — | No | — |
| | 21:16:00 | | | | | |
| 1196 | 3 Jul. 2018 | No | — | — | No | — |
| | 21:46:00 | | | | | |
| 1196 | 4 Jul. 2018 | Yes | Moderate | — | Yes | 1 |
| | 21:48:00 | | | | | |
| 1196 | 7 Jul. 2018 | Yes | Severe | — | Yes | 1 |
| | 21:23:00 | | | | | |
| 1196 | 8 Jul. 2018 | No | — | — | No | — |
| | 21:02:00 | | | | | |
| 1196 | 9 Jul. 2018 | No | — | — | No | — |
| | 22:30:00 | | | | | |
| 1196 | 10 Jul. 2018 | No | — | — | No | — |
| | 21:30:00 | | | | | |
| 1196 | 11 Jul. 2018 | Yes | Moderate | — | Yes | 1 |
| | 21:17:00 | | | | | |
| 1196 | 12 Jul. 2018 | No | — | — | No | — |
| | 21:35:00 | | | | | |
| 1196 | 13 Jul. 2018 | No | — | — | No | — |
| | 22:28:00 | | | | | |
| 1196 | 15 Jul. 2018 | No | — | — | No | — |
| | 21:15:00 | | | | | |
| 1196 | 16 Jul. 2018 | Yes | Moderate | — | Yes | 1 |
| | 21:14:00 | | | | | |
| 1196 | 17 Jul. 2018 | No | — | — | No | — |
| | 21:12:00 | | | | | |
| 1196 | 18 Jul. 2018 | No | — | — | No | — |
| | 20:33:00 | | | | | |
| 1196 | 19 Jul. 2018 | No | — | — | No | — |
| | 21:33:00 | | | | | |
| 1196 | 20 Jul. 2018 | Yes | Severe | — | Yes | 1 |
| | 21:10:00 | | | | | |
| 1196 | 21 Jul. 2018 | No | — | — | No | — |
| | 21:31:00 | | | | | |
| 1196 | 22 Jul. 2018 | No | — | — | No | — |
| | 20:41:00 | | | | | |
| 1196 | 23 Jul. 2018 | No | — | — | No | — |
| | 11:48:00 | | | | | |
| 1196 | 23 Jul. 2018 | Yes | Moderate | — | Yes | 1 |
| | 12:48:00 | | | | | |

Subject 1196 is a 50 year-old Caucasian female with history of 2-8 migraine attacks (without aura) per month since the age of 25. Past medication used to treat her headache has been sumatriptan, eletriptan, ibuprofen, paracetamol, hydrocodone tritartrate, and methylprednisolone. Additionally, she has been receiving erenumab-aooe injections from 23 Jul. 2018 and Botox® injections from 26 May 2017 to 0 May 2018.

TABLE 3

| | | | | Take other | Take study | |
|---|---|---|---|---|---|---|
| | | | | medication | medication | |
| | | Have | | [Observation | [Treatment | Number of |
| Subject ID | Report Datetime | migraine | Severity | phase] | phase] | tablets |
| 2099 | 12 Apr. 2018 | Yes | Moderate | Yes | — | — |
| | 18:17:00 | | | | | |
| 2099 | 13 Apr. 2018 | Yes | Severe | Yes | — | — |
| | 18:16:00 | | | | | |
| 2099 | 14 Apr. 2018 | Yes | Moderate | Yes | — | — |
| | 18:03:00 | | | | | |
| 2099 | 15 Apr. 2018 | Yes | Mild | No | — | — |
| | 18:03:00 | | | | | |
| 2099 | 16 Apr. 2018 | Yes | Moderate | Yes | — | — |
| | 19:20:00 | | | | | |
| 2099 | 17 Apr. 2018 | No | — | — | — | — |
| | 19:27:00 | | | | | |
| 2099 | 18 Apr. 2018 | Yes | Severe | Yes | — | — |
| | 19:51:00 | | | | | |
| 2099 | 19 Apr. 2018 | Yes | Severe | Yes | — | — |
| | 19:05:00 | | | | | |
| 2099 | 20 Apr. 2018 | Yes | Severe | Yes | — | — |
| | 18:22:00 | | | | | |
| 2099 | 21 Apr. 2018 | Yes | Mild | Yes | — | — |
| | 18:45:00 | | | | | |
| 2099 | 22 Apr. 2018 | Yes | Moderate | Yes | — | — |
| | 18:13:00 | | | | | |
| 2099 | 23 Apr. 2018 | No | — | — | — | — |
| | 18:30:00 | | | | | |
| 2099 | 24 Apr. 2018 | Yes | Mild | Yes | — | — |
| | 21:45:00 | | | | | |
| 2099 | 25 Apr. 2018 | No | — | — | — | — |
| | 19:54:00 | | | | | |
| 2099 | 26 Apr. 2018 | No | — | — | — | — |
| | 22:32:00 | | | | | |
| 2099 | 27 Apr. 2018 | Yes | Mild | Yes | — | — |
| | 18:04:00 | | | | | |
| 2099 | 28 Apr. 2018 | No | — | — | — | — |
| | 18:05:00 | | | | | |
| 2099 | 1 May 2018 | Yes | Mild | No | — | — |
| | 18:00:00 | | | | | |
| 2099 | 2 May 2018 | Yes | Moderate | Yes | — | — |
| | 18:02:00 | | | | | |
| 2099 | 3 May 2018 | No | — | — | — | — |
| | 18:02:00 | | | | | |
| 2099 | 4 May 2018 | Yes | Moderate | Yes | — | — |
| | 18:28:00 | | | | | |
| 2099 | 5 May 2018 | No | — | — | — | — |
| | 18:02:00 | | | | | |
| 2099 | 6 May 2018 | Yes | Severe | Yes | — | — |
| | 18:01:00 | | | | | |
| 2099 | 7 May 2018 | No | — | — | — | — |
| | 19:21:00 | | | | | |
| 2099 | 8 May 2018 | Yes | Severe | Yes | — | — |
| | 19:18:00 | | | | | |
| 2099 | 9 May 2018 | Yes | Mild | Yes | — | — |
| | 19:55:00 | | | | | |
| 2099 | 10 May 2018 | No | — | — | No | — |
| | 23:31:00 | | | | | |
| 2099 | 11 May 2018 | Yes | Moderate | — | Yes | 1 |
| | 19:43:00 | | | | | |
| 2099 | 12 May 2018 | Yes | Severe | — | Yes | 1 |
| | 18:01:00 | | | | | |
| 2099 | 13 May 2018 | Yes | Mild | — | Yes | 1 |
| | 18:02:00 | | | | | |
| 2099 | 14 May 2018 | Yes | Mild | — | Yes | 1 |
| | 18:01:00 | | | | | |
| 2099 | 15 May 2018 | No | — | — | No | — |
| | 21:30:00 | | | | | |
| 2099 | 16 May 2018 | No | — | — | No | — |
| | 23:33:00 | | | | | |
| 2099 | 17 May 2018 | No | — | — | No | — |
| | 19:37:00 | | | | | |
| 2099 | 18 May 2018 | Yes | Mild | — | Yes | 1 |
| | 18:30:00 | | | | | |
| 2099 | 19 May 2018 | Yes | Mild | — | Yes | 1 |
| | 18:04:00 | | | | | |
| 2099 | 21 May 2018 | Yes | Mild | — | Yes | 1 |
| | 19:17:00 | | | | | |
| 2099 | 22 May 2018 | No | — | — | No | — |
| | 23:19:00 | | | | | |
| 2099 | 23 May 2018 | Yes | Severe | — | Yes | 1 |
| | 20:13:00 | | | | | |
| 2099 | 24 May 2018 | No | — | — | No | — |
| | 21:22:00 | | | | | |
| 2099 | 25 May 2018 | Yes | Moderate | — | Yes | 1 |
| | 18:16:00 | | | | | |
| 2099 | 26 May 2018 | Yes | Moderate | — | Yes | 1 |
| | 18:00:00 | | | | | |
| 2099 | 27 May 2018 | No | — | — | No | — |
| | 18:32:00 | | | | | |
| 2099 | 28 May 2018 | Yes | Moderate | — | Yes | 1 |
| | 18:45:00 | | | | | |
| 2099 | 29 May 2018 | No | — | — | No | — |
| | 20:59:00 | | | | | |
| 2099 | 30 May 2018 | Yes | Severe | — | Yes | 1 |
| | 19:36:00 | | | | | |
| 2099 | 31 May 2018 | No | — | — | No | — |
| | 18:42:00 | | | | | |
| 2099 | 3 Jun. 2018 | Yes | Moderate | — | Yes | 1 |
| | 18:59:00 | | | | | |
| 2099 | 4 Jun. 2018 | No | — | — | No | — |
| | 19:37:00 | | | | | |
| 2099 | 5 Jun. 2018 | No | — | — | No | — |
| | 19:53:00 | | | | | |
| 2099 | 6 Jun. 2018 | No | — | — | No | — |
| | 19:28:00 | | | | | |
| 2099 | 7 Jun. 2018 | Yes | Moderate | — | Yes | 1 |
| | 18:19:00 | | | | | |
| 2099 | 8 Jun. 2018 | No | — | — | No | — |
| | 18:19:00 | | | | | |
| 2099 | 9 Jun. 2018 | Yes | Moderate | — | Yes | 1 |
| | 18:04:00 | | | | | |
| 2099 | 10 Jun. 2018 | Yes | Mild | — | Yes | 1 |
| | 18:16:00 | | | | | |
| 2099 | 11 Jun. 2018 | No | — | — | No | — |
| | 19:21:00 | | | | | |
| 2099 | 13 Jun. 2018 | No | — | — | No | — |
| | 19:36:00 | | | | | |
| 2099 | 14 Jun. 2018 | Yes | Mild | — | Yes | 1 |
| | 19:41:00 | | | | | |
| 2099 | 15 Jun. 2018 | No | — | — | No | — |
| | 23:33:00 | | | | | |
| 2099 | 16 Jun. 2018 | No | — | — | No | — |
| | 18:00:00 | | | | | |
| 2099 | 17 Jun. 2018 | Yes | Severe | — | Yes | 1 |
| | 18:15:00 | | | | | |
| 2099 | 18 Jun. 2018 | No | — | — | No | — |
| | 20:57:00 | | | | | |
| 2099 | 19 Jun. 2018 | No | — | — | No | — |
| | 19:32:00 | | | | | |
| 2099 | 20 Jun. 2018 | No | — | — | No | — |
| | 18:32:00 | | | | | |
| 2099 | 21 Jun. 2018 | No | — | — | No | — |
| | 19:30:00 | | | | | |
| 2099 | 23 Jun. 2018 | No | — | — | No | — |
| | 18:03:00 | | | | | |
| 2099 | 24 Jun. 2018 | No | — | — | No | — |
| | 18:36:00 | | | | | |
| 2099 | 25 Jun. 2018 | No | — | — | No | — |
| | 18:32:00 | | | | | |
| 2099 | 26 Jun. 2018 | No | — | — | No | — |
| | 19:01:00 | | | | | |
| 2099 | 27 Jun. 2018 | No | — | — | No | — |
| | 18:12:00 | | | | | |
| 2099 | 29 Jun. 2018 | No | — | — | No | — |
| | 19:13:00 | | | | | |
| 2099 | 30 Jun. 2018 | Yes | Severe | — | Yes | 1 |
| | 23:42:00 | | | | | |
| 2099 | 1 Jul. 2018 | Yes | Moderate | — | Yes | 1 |
| | 18:01:00 | | | | | |
| 2099 | 3 Jul. 2018 | No | — | — | No | — |
| | 18:02:00 | | | | | |
| 2099 | 4 Jul. 2018 | Yes | Severe | — | Yes | 1 |
| | 22:28:00 | | | | | |
| 2099 | 5 Jul. 2018 | No | — | — | No | — |
| | 18:33:00 | | | | | |
| 2099 | 6 Jul. 2018 | No | — | — | No | — |
| | 18:11:00 | | | | | |
| 2099 | 7 Jul. 2018 | No | — | — | No | — |
| | 23:31:00 | | | | | |
| 2099 | 8 Jul. 2018 | No | — | — | No | — |
| | 18:20:00 | | | | | |
| 2099 | 9 Jul. 2018 | No | — | — | No | — |
| | 18:32:00 | | | | | |
| 2099 | 10 Jul. 2018 | Yes | Severe | — | Yes | 1 |
| | 19:55:00 | | | | | |
| 2099 | 11 Jul. 2018 | No | — | — | No | — |
| | 20:21:00 | | | | | |
| 2099 | 13 Jul. 2018 | No | — | — | No | — |
| | 22:08:00 | | | | | |
| 2099 | 15 Jul. 2018 | No | — | — | No | — |
| | 19:46:00 | | | | | |
| 2099 | 16 Jul. 2018 | Yes | Mild | — | Yes | 1 |
| | 18:16:00 | | | | | |
| 2099 | 17 Jul. 2018 | No | — | — | No | — |
| | 18:26:00 | | | | | |
| 2099 | 18 Jul. 2018 | No | — | — | No | — |
| | 21:06:00 | | | | | |
| 2099 | 19 Jul. 2018 | Yes | Severe | — | Yes | 1 |
| | 19:39:00 | | | | | |
| 2099 | 20 Jul. 2018 | Yes | Moderate | — | Yes | 1 |
| | 18:01:00 | | | | | |
| 2099 | 21 Jul. 2018 | No | — | — | No | — |
| | 18:16:00 | | | | | |
| 2099 | 22 Jul. 2018 | No | — | — | No | — |
| | 19:53:00 | | | | | |
| 2099 | 23 Jul. 2018 | Yes | Mild | — | Yes | 1 |
| | 19:14:00 | | | | | |
| 2099 | 24 Jul. 2018 | No | — | — | No | — |
| | 20:02:00 | | | | | |
| 2099 | 25 Jul. 2018 | Yes | Mild | — | Yes | 1 |
| | 20:55:00 | | | | | |
| 2099 | 26 Jul. 2018 | No | — | — | No | — |
| | 09:16:00 | | | | | |

Subject 2099 is a 53 year-old Caucasian male with history of 9-14 migraine attacks per month, who has been receiving erenumab-aooe injections.

TABLE 4

| | | | | Take other | Take study | |
|---|---|---|---|---|---|---|
| | | | | medication | medication | |
| | | Have | | [Observation | [Treatment | Number of |
| Subject ID | Report Datetime | migraine | Severity | phase] | phase] | tablets |
| 2120 | 17 Apr. 2018 | No | — | — | — | — |
| | 18:01:00 | | | | | |
| 2120 | 18 Apr. 2018 | Yes | Moderate | Yes | — | — |
| | 18:00:00 | | | | | |
| 2120 | 19 Apr. 2018 | No | — | — | — | — |
| | 18:00:00 | | | | | |
| 2120 | 20 Apr. 2018 | Yes | Moderate | Yes | — | — |
| | 18:02:00 | | | | | |
| 2120 | 21 Apr. 2018 | No | — | — | — | — |
| | 18:00:00 | | | | | |
| 2120 | 22 Apr. 2018 | Yes | Severe | Yes | — | — |
| | 18:00:00 | | | | | |
| 2120 | 23 Apr. 2018 | Yes | Moderate | Yes | — | — |
| | 18:02:00 | | | | | |
| 2120 | 24 Apr. 2018 | No | — | — | — | — |
| | 18:02:00 | | | | | |
| 2120 | 25 Apr. 2018 | Yes | Moderate | Yes | — | — |
| | 18:00:00 | | | | | |
| 2120 | 26 Apr. 2018 | No | — | — | — | — |
| | 18:02:00 | | | | | |
| 2120 | 27 Apr. 2018 | No | — | — | — | — |
| | 18:00:00 | | | | | |
| 2120 | 29 Apr. 2018 | Yes | Moderate | Yes | — | — |
| | 19:26:00 | | | | | |
| 2120 | 30 Apr. 2018 | No | — | — | — | — |
| | 18:33:00 | | | | | |
| 2120 | 1 May 2018 | Yes | Moderate | Yes | — | — |
| | 18:01:00 | | | | | |
| 2120 | 2 May 2018 | Yes | Moderate | Yes | — | — |
| | 18:01:00 | | | | | |
| 2120 | 3 May 2018 | No | — | — | — | — |
| | 18:00:00 | | | | | |
| 2120 | 4 May 2018 | No | — | — | — | — |
| | 18:00:00 | | | | | |
| 2120 | 5 May 2018 | No | — | — | — | — |
| | 18:00:00 | | | | | |
| 2120 | 6 May 2018 | Yes | Severe | Yes | — | — |
| | 18:00:00 | | | | | |
| 2120 | 7 May 2018 | No | — | — | — | — |
| | 18:02:00 | | | | | |
| 2120 | 8 May 2018 | No | — | — | — | — |
| | 18:01:00 | | | | | |
| 2120 | 9 May 2018 | No | — | — | — | — |
| | 18:02:00 | | | | | |
| 2120 | 10 May 2018 | Yes | Severe | Yes | — | — |
| | 18:00:00 | | | | | |
| 2120 | 11 May 2018 | Yes | Moderate | Yes | — | — |
| | 18:01:00 | | | | | |
| 2120 | 12 May 2018 | No | — | — | — | — |
| | 18:01:00 | | | | | |
| 2120 | 13 May 2018 | Yes | Mild | No | — | — |
| | 18:01:00 | | | | | |
| 2120 | 14 May 2018 | No | — | — | — | — |
| | 18:01:00 | | | | | |
| 2120 | 15 May 2018 | No | — | — | — | — |
| | 18:00:00 | | | | | |
| 2120 | 16 May 2018 | Yes | Mild | — | No | — |
| | 18:01:00 | | | | | |
| 2120 | 17 May 2018 | No | — | — | No | — |
| | 18:00:00 | | | | | |
| 2120 | 18 May 2018 | No | — | — | No | — |
| | 18:00:00 | | | | | |
| 2120 | 19 May 2018 | Yes | Moderate | — | No | — |
| | 18:01:00 | | | | | |
| 2120 | 20 May 2018 | No | — | — | No | — |
| | 18:00:00 | | | | | |
| 2120 | 21 May 2018 | Yes | Moderate | — | No | — |
| | 18:11:00 | | | | | |
| 2120 | 22 May 2018 | No | — | — | No | — |
| | 18:05:00 | | | | | |
| 2120 | 23 May 2018 | Yes | Moderate | — | Yes | 1 |
| | 18:03:00 | | | | | |
| 2120 | 24 May 2018 | No | — | — | No | — |
| | 18:02:00 | | | | | |
| 2120 | 25 May 2018 | Yes | Moderate | — | Yes | 1 |
| | 18:00:00 | | | | | |
| 2120 | 26 May 2018 | No | — | — | No | — |
| | 18:19:00 | | | | | |
| 2120 | 27 May 2018 | Yes | Moderate | — | Yes | 1 |
| | 18:00:00 | | | | | |
| 2120 | 28 May 2018 | Yes | Moderate | — | Yes | 1 |
| | 18:00:00 | | | | | |
| 2120 | 29 May 2018 | No | — | — | No | — |
| | 18:01:00 | | | | | |
| 2120 | 31 May 2018 | Yes | Moderate | — | Yes | 1 |
| | 18:00:00 | | | | | |
| 2120 | 1 Jun. 2018 | Yes | Moderate | — | Yes | 1 |
| | 18:22:00 | | | | | |
| 2120 | 2 Jun. 2018 | Yes | Mild | — | No | — |
| | 18:00:00 | | | | | |
| 2120 | 3 Jun. 2018 | No | — | — | No | — |
| | 18:01:00 | | | | | |
| 2120 | 4 Jun. 2018 | Yes | Moderate | — | Yes | 1 |
| | 18:00:00 | | | | | |
| 2120 | 5 Jun. 2018 | Yes | Moderate | — | Yes | 1 |
| | 18:00:00 | | | | | |
| 2120 | 6 Jun. 2018 | Yes | Mild | — | Yes | 1 |
| | 18:02:00 | | | | | |
| 2120 | 7 Jun. 2018 | Yes | Moderate | — | Yes | 1 |
| | 18:00:00 | | | | | |
| 2120 | 8 Jun. 2018 | No | — | — | No | — |
| | 18:00:00 | | | | | |
| 2120 | 10 Jun. 2018 | Yes | Moderate | — | Yes | 1 |
| | 18:01:00 | | | | | |
| 2120 | 11 Jun. 2018 | Yes | Mild | — | Yes | 1 |
| | 18:01:00 | | | | | |
| 2120 | 12 Jun. 2018 | Yes | Moderate | — | Yes | 1 |
| | 18:00:00 | | | | | |
| 2120 | 13 Jun. 2018 | Yes | Mild | — | Yes | 1 |
| | 18:03:00 | | | | | |
| 2120 | 14 Jun. 2018 | Yes | Moderate | — | Yes | 1 |
| | 18:05:00 | | | | | |
| 2120 | 15 Jun. 2018 | Yes | Mild | — | Yes | 1 |
| | 22:53:00 | | | | | |
| 2120 | 16 Jun. 2018 | Yes | Moderate | — | Yes | 1 |
| | 18:01:00 | | | | | |
| 2120 | 17 Jun. 2018 | No | — | — | No | — |
| | 18:00:00 | | | | | |
| 2120 | 18 Jun. 2018 | No | — | — | No | — |
| | 18:01:00 | | | | | |
| 2120 | 19 Jun. 2018 | Yes | Moderate | — | Yes | 1 |
| | 18:00:00 | | | | | |
| 2120 | 20 Jun. 2018 | No | — | — | No | — |
| | 18:01:00 | | | | | |
| 2120 | 21 Jun. 2018 | Yes | Moderate | — | Yes | 1 |
| | 18:05:00 | | | | | |
| 2120 | 22 Jun. 2018 | Yes | Moderate | — | Yes | 1 |
| | 18:01:00 | | | | | |
| 2120 | 23 Jun. 2018 | Yes | Severe | — | Yes | 1 |
| | 20:15:00 | | | | | |
| 2120 | 24 Jun. 2018 | Yes | Moderate | — | Yes | 1 |
| | 18:00:00 | | | | | |
| 2120 | 25 Jun. 2018 | Yes | Mild | — | Yes | 1 |
| | 18:00:00 | | | | | |
| 2120 | 26 Jun. 2018 | No | — | — | No | — |
| | 19:55:00 | | | | | |
| 2120 | 27 Jun. 2018 | Yes | Mild | — | Yes | 1 |
| | 18:00:00 | | | | | |
| 2120 | 28 Jun. 2018 | Yes | Moderate | — | Yes | 1 |
| | 18:00:00 | | | | | |
| 2120 | 29 Jun. 2018 | Yes | Moderate | — | Yes | 1 |
| | 18:02:00 | | | | | |
| 2120 | 30 Jun. 2018 | No | — | — | No | — |
| | 18:00:00 | | | | | |
| 2120 | 1 Jul. 2018 | Yes | Mild | — | Yes | 1 |
| | 18:34:00 | | | | | |
| 2120 | 2 Jul. 2018 | Yes | Moderate | — | Yes | 1 |
| | 18:00:00 | | | | | |
| 2120 | 3 Jul. 2018 | Yes | Severe | — | Yes | 1 |
| | 19:15:00 | | | | | |
| 2120 | 4 Jul. 2018 | Yes | Moderate | — | Yes | 1 |
| | 18:27:00 | | | | | |
| 2120 | 5 Jul. 2018 | Yes | Moderate | — | Yes | 1 |
| | 18:30:00 | | | | | |
| 2120 | 6 Jul. 2018 | No | — | — | No | — |
| | 18:00:00 | | | | | |
| 2120 | 7 Jul. 2018 | Yes | Moderate | — | Yes | 1 |
| | 18:15:00 | | | | | |
| 2120 | 8 Jul. 2018 | No | — | — | No | — |
| | 18:00:00 | | | | | |
| 2120 | 9 Jul. 2018 | Yes | Mild | — | Yes | 1 |
| | 20:51:00 | | | | | |
| 2120 | 10 Jul. 2018 | Yes | Moderate | — | Yes | 1 |
| | 18:17:00 | | | | | |
| 2120 | 11 Jul. 2018 | No | — | — | No | — |
| | 18:00:00 | | | | | |
| 2120 | 12 Jul. 2018 | Yes | Moderate | — | Yes | 1 |
| | 18:03:00 | | | | | |
| 2120 | 13 Jul. 2018 | No | — | — | No | — |
| | 18:51:00 | | | | | |
| 2120 | 14 Jul. 2018 | Yes | Severe | — | Yes | 1 |
| | 21:48:00 | | | | | |
| 2120 | 15 Jul. 2018 | No | — | — | No | — |
| | 21:06:00 | | | | | |
| 2120 | 16 Jul. 2018 | No | — | — | No | — |
| | 23:23:00 | | | | | |
| 2120 | 17 Jul. 2018 | Yes | Severe | — | Yes | 1 |
| | 18:00:00 | | | | | |
| 2120 | 18 Jul. 2018 | No | — | — | No | — |
| | 18:00:00 | | | | | |
| 2120 | 19 Jul. 2018 | No | — | — | No | — |
| | 18:00:00 | | | | | |
| 2120 | 21 Jul. 2018 | Yes | Mild | — | Yes | 1 |
| | 20:15:00 | | | | | |
| 2120 | 22 Jul. 2018 | Yes | Moderate | — | No | — |
| | 19:01:00 | | | | | |
| 2120 | 23 Jul. 2018 | Yes | Moderate | — | Yes | 1 |
| | 18:00:00 | | | | | |
| 2120 | 24 Jul. 2018 | Yes | Severe | — | Yes | 1 |
| | 18:05:00 | | | | | |
| 2120 | 25 Jul. 2018 | No | — | — | No | — |
| | 22:50:00 | | | | | |
| 2120 | 26 Jul. 2018 | No | — | — | No | — |
| | 18:01:00 | | | | | |
| 2120 | 27 Jul. 2018 | Yes | Moderate | — | Yes | 1 |
| | 18:00:00 | | | | | |
| 2120 | 28 Jul. 2018 | Yes | Moderate | — | Yes | 1 |
| | 18:45:00 | | | | | |
| 2120 | 29 Jul. 2018 | Yes | Moderate | — | Yes | 1 |
| | 20:51:00 | | | | | |
| 2120 | 30 Jul. 2018 | Yes | Mild | — | Yes | 1 |
| | 18:02:00 | | | | | |
| 2120 | 31 Jul. 2018 | Yes | Mild | — | Yes | 1 |
| | 18:01:00 | | | | | |
| 2120 | 1 Aug. 2018 | Yes | Mild | — | Yes | 1 |
| | 18:21:00 | | | | | |
| 2120 | 2 Aug. 2018 | Yes | Mild | — | Yes | 1 |
| | 18:01:00 | | | | | |
| 2120 | 3 Aug. 2018 | Yes | Mild | — | Yes | 1 |
| | 18:00:00 | | | | | |
| 2120 | 4 Aug. 2018 | Yes | Mild | — | Yes | 1 |
| | 18:16:00 | | | | | |
| 2120 | 6 Aug. 2018 | Yes | Mild | — | Yes | 1 |
| | 20:12:00 | | | | | |
| 2120 | 7 Aug. 2018 | No | — | — | No | — |
| | 18:01:00 | | | | | |
| 2120 | 8 Aug. 2018 | Yes | Severe | — | Yes | 1 |
| | 18:01:00 | | | | | |
| 2120 | 9 Aug. 2018 | Yes | Mild | — | Yes | 1 |
| | 21:11:00 | | | | | |
| 2120 | 11 Aug. 2018 | Yes | Mild | — | Yes | 1 |
| | 18:16:00 | | | | | |
| 2120 | 12 Aug. 2018 | Yes | Mild | — | Yes | 1 |
| | 18:47:00 | | | | | |
| 2120 | 13 Aug. 2018 | Yes | Mild | — | Yes | 1 |
| | 22:28:00 | | | | | |
| 2120 | 14 Aug. 2018 | No | — | — | No | — |
| | 18:00:00 | | | | | |
| 2120 | 15 Aug. 2018 | Yes | Moderate | — | Yes | 1 |
| | 18:01:00 | | | | | |
| 2120 | 16 Aug. 2018 | No | — | — | No | — |
| | 18:03:00 | | | | | |
| 2120 | 17 Aug. 2018 | Yes | Mild | — | Yes | 1 |
| | 18:02:00 | | | | | |
| 2120 | 18 Aug. 2018 | Yes | Mild | — | Yes | 1 |
| | 20:37:00 | | | | | |
| 2120 | 19 Aug. 2018 | Yes | Moderate | — | Yes | 1 |
| | 18:16:00 | | | | | |
| 2120 | 20 Aug. 2018 | Yes | Mild | — | Yes | 1 |
| | 18:21:00 | | | | | |
| 2120 | 21 Aug. 2018 | No | — | — | No | — |
| | 18:28:00 | | | | | |
| 2120 | 22 Aug. 2018 | Yes | Mild | — | Yes | 1 |
| | 19:40:00 | | | | | |
| 2120 | 23 Aug. 2018 | No | — | — | No | — |
| | 18:02:00 | | | | | |
| 2120 | 25 Aug. 2018 | Yes | Severe | — | Yes | 1 |
| | 18:02:00 | | | | | |
| 2120 | 26 Aug. 2018 | Yes | Mild | — | Yes | 1 |
| | 18:03:00 | | | | | |
| 2120 | 27 Aug. 2018 | No | — | — | No | — |
| | 18:00:00 | | | | | |
| 2120 | 28 Aug. 2018 | Yes | Moderate | — | Yes | 1 |
| | 18:01:00 | | | | | |
| 2120 | 29 Aug. 2018 | Yes | Mild | — | No | — |
| | 18:02:00 | | | | | |
| 2120 | 30 Aug. 2018 | Yes | Mild | — | Yes | 1 |
| | 18:15:00 | | | | | |
| 2120 | 31 Aug. 2018 | Yes | Mild | — | Yes | 1 |
| | 18:00:00 | | | | | |
| 2120 | 1 Sep. 2018 | Yes | Moderate | — | Yes | 1 |
| | 18:01:00 | | | | | |
| 2120 | 2 Sep. 2018 | No | — | — | No | — |
| | 19:52:00 | | | | | |
| 2120 | 3 Sep. 2018 | Yes | Moderate | — | Yes | 1 |
| | 22:59:00 | | | | | |
| 2120 | 4 Sep. 2018 | No | — | — | No | — |
| | 22:30:00 | | | | | |
| 2120 | 5 Sep. 2018 | Yes | Mild | — | Yes | 1 |
| | 23:18:00 | | | | | |
| 2120 | 7 Sep. 2018 | Yes | Moderate | — | Yes | 1 |
| | 23:18:00 | | | | | |
| 2120 | 8 Sep. 2018 | No | — | — | No | — |
| | 22:15:00 | | | | | |
| 2120 | 10 Sep. 2018 | Yes | Moderate | — | Yes | 1 |
| | 22:51:00 | | | | | |
| 2120 | 11 Sep. 2018 | Yes | Moderate | — | Yes | 1 |
| | 16:32:00 | | | | | |

Subject 2120 is a 62 year-old Caucasian male with history of 9-14 migraine attacks per month, who has been receiving erenumab-aooe injections.

TABLE 5

| | | | | Take other | Take study | |
|---|---|---|---|---|---|---|
| | | | | medication | medication | |
| | | Have | | [Observation | [Treatment | Number of |
| Subject ID | Report Datetime | migraine | Severity | phase] | phase] | tablets |
| 2637 | 3 Aug. 2018 | No | — | — | — | — |
| | 18:03:00 | | | | | |
| 2637 | 4 Aug. 2018 | No | — | — | — | — |
| | 18:07:00 | | | | | |
| 2637 | 5 Aug. 2018 | Yes | Severe | Yes | — | — |
| | 18:02:00 | | | | | |
| 2637 | 6 Aug. 2018 | No | — | — | — | — |
| | 18:01:00 | | | | | |
| 2637 | 7 Aug. 2018 | No | — | — | — | — |
| | 18:01:00 | | | | | |
| 2637 | 8 Aug. 2018 | Yes | Moderate | No | — | — |
| | 18:12:00 | | | | | |
| 2637 | 9 Aug. 2018 | No | — | — | — | — |
| | 18:01:00 | | | | | |
| 2637 | 10 Aug. 2018 | Yes | Moderate | No | — | — |
| | 18:02:00 | | | | | |
| 2637 | 11 Aug. 2018 | No | — | — | — | — |
| | 18:01:00 | | | | | |
| 2637 | 12 Aug. 2018 | No | — | — | — | — |
| | 18:01:00 | | | | | |
| 2637 | 13 Aug. 2018 | No | — | — | — | — |
| | 18:01:00 | | | | | |
| 2637 | 14 Aug. 2018 | No | — | — | — | — |
| | 18:01:00 | | | | | |
| 2637 | 15 Aug. 2018 | No | — | — | — | — |
| | 18:01:00 | | | | | |
| 2637 | 16 Aug. 2018 | Yes | Severe | Yes | — | — |
| | 18:01:00 | | | | | |
| 2637 | 17 Aug. 2018 | No | — | — | — | — |
| | 18:01:00 | | | | | |
| 2637 | 18 Aug. 2018 | Yes | Severe | Yes | — | — |
| | 18:01:00 | | | | | |
| 2637 | 19 Aug. 2018 | Yes | Severe | No | — | — |
| | 18:09:00 | | | | | |
| 2637 | 20 Aug. 2018 | No | — | — | — | — |
| | 18:00:00 | | | | | |
| 2637 | 21 Aug. 2018 | No | — | — | — | — |
| | 18:01:00 | | | | | |
| 2637 | 22 Aug. 2018 | No | — | — | — | — |
| | 18:00:00 | | | | | |
| 2637 | 23 Aug. 2018 | No | — | — | — | — |
| | 18:00:00 | | | | | |
| 2637 | 24 Aug. 2018 | No | — | — | — | — |
| | 18:01:00 | | | | | |
| 2637 | 25 Aug. 2018 | No | — | — | — | — |
| | 18:32:00 | | | | | |
| 2637 | 26 Aug. 2018 | Yes | Severe | No | — | — |
| | 19:25:00 | | | | | |
| 2637 | 27 Aug. 2018 | No | — | — | — | — |
| | 18:01:00 | | | | | |
| 2637 | 28 Aug. 2018 | No | — | — | — | — |
| | 18:01:00 | | | | | |
| 2637 | 29 Aug. 2018 | No | — | — | — | — |
| | 18:01:00 | | | | | |
| 2637 | 30 Aug. 2018 | No | — | — | — | — |
| | 18:01:00 | | | | | |
| 2637 | 31 Aug. 2018 | No | — | — | No | — |
| | 18:01:00 | | | | | |
| 2637 | 1 Sep. 2018 | No | — | — | No | — |
| | 18:03:00 | | | | | |
| 2637 | 2 Sep. 2018 | No | — | — | No | — |
| | 18:01:00 | | | | | |
| 2637 | 3 Sep. 2018 | No | — | — | No | — |
| | 19:44:00 | | | | | |
| 2637 | 4 Sep. 2018 | No | — | — | No | — |
| | 18:01:00 | | | | | |
| 2637 | 5 Sep. 2018 | No | — | — | No | — |
| | 18:00:00 | | | | | |
| 2637 | 6 Sep. 2018 | Yes | Severe | — | Yes | 1 |
| | 18:00:00 | | | | | |
| 2637 | 7 Sep. 2018 | No | — | — | No | — |
| | 18:03:00 | | | | | |
| 2637 | 8 Sep. 2018 | Yes | Moderate | — | Yes | 1 |
| | 22:11:00 | | | | | |
| 2637 | 9 Sep. 2018 | No | — | — | No | — |
| | 18:00:00 | | | | | |
| 2637 | 10 Sep. 2018 | Yes | Severe | — | Yes | 1 |
| | 18:00:00 | | | | | |
| 2637 | 11 Sep. 2018 | No | — | — | No | — |
| | 18:01:00 | | | | | |
| 2637 | 12 Sep. 2018 | Yes | Severe | — | Yes | 1 |
| | 18:01:00 | | | | | |
| 2637 | 13 Sep. 2018 | Yes | Severe | — | Yes | 1 |
| | 18:00:00 | | | | | |
| 2637 | 14 Sep. 2018 | Yes | Severe | — | Yes | 1 |
| | 12:10:00 | | | | | |

Subject 2637 is a 72 year-old Caucasian female who has been receiving erenumab-aooe injections.

TABLE 6

| | | | | Take other | Take study | |
|---|---|---|---|---|---|---|
| | | | | medication | medication | Number |
| Subject | | Have | | [Observation | [Treatment | of |
| ID | Report Datetime | migraine | Severity | phase] | phase] | tablets |
| 1990 | 27 Mar. 2018 | Yes | Severe | Yes | — | — |
| | 18:41:00 | | | | | |
| 1990 | 28 Mar. 2018 | Yes | Severe | Yes | — | — |
| | 18:19:00 | | | | | |
| 1990 | 29 Mar. 2018 | No | — | — | — | — |
| | 19:08:00 | | | | | |
| 1990 | 30 Mar. 2018 | Yes | Moderate | Yes | — | — |
| | 18:15:00 | | | | | |
| 1990 | 31 Mar. 2018 | No | — | — | — | — |
| | 18:00:00 | | | | | |
| 1990 | 1 Apr. 2018 | Yes | Mild | Yes | — | — |
| | 18:48:00 | | | | | |
| 1990 | 2 Apr. 2018 | Yes | Moderate | Yes | — | — |
| | 18:18:00 | | | | | |
| 1990 | 3 Apr. 2018 | Yes | Mild | Yes | — | — |
| | 18:16:00 | | | | | |
| 1990 | 4 Apr. 2018 | No | — | — | — | — |
| | 20:14:00 | | | | | |
| 1990 | 5 Apr. 2018 | No | — | — | — | — |
| | 18:01:00 | | | | | |
| 1990 | 6 Apr. 2018 | Yes | Moderate | Yes | — | — |
| | 18:03:00 | | | | | |
| 1990 | 7 Apr. 2018 | No | — | — | — | — |
| | 18:02:00 | | | | | |
| 1990 | 8 Apr. 2018 | Yes | Moderate | Yes | — | — |
| | 19:21:00 | | | | | |
| 1990 | 9 Apr. 2018 | No | — | — | — | — |
| | 18:00:00 | | | | | |
| 1990 | 10 Apr. 2018 | Yes | Moderate | Yes | — | — |
| | 21:16:00 | | | | | |
| 1990 | 11 Apr. 2018 | No | — | — | — | — |
| | 18:17:00 | | | | | |
| 1990 | 12 Apr. 2018 | Yes | Mild | Yes | — | — |
| | 18:47:00 | | | | | |
| 1990 | 14 Apr. 2018 | No | — | — | — | — |
| | 18:20:00 | | | | | |
| 1990 | 15 Apr. 2018 | Yes | Mild | Yes | — | — |
| | 18:32:00 | | | | | |
| 1990 | 16 Apr. 2018 | Yes | Moderate | Yes | — | — |
| | 18:21:00 | | | | | |
| 1990 | 17 Apr. 2018 | No | — | — | — | — |
| | 18:01:00 | | | | | |
| 1990 | 18 Apr. 2018 | Yes | Moderate | Yes | — | — |
| | 18:01:00 | | | | | |
| 1990 | 19 Apr. 2018 | No | — | — | — | — |
| | 18:06:00 | | | | | |
| 1990 | 20 Apr. 2018 | Yes | Moderate | Yes | — | — |
| | 18:02:00 | | | | | |
| 1990 | 21 Apr. 2018 | No | — | — | — | — |
| | 18:50:00 | | | | | |
| 1990 | 22 Apr. 2018 | No | — | — | — | — |
| | 18:02:00 | | | | | |
| 1990 | 24 Apr. 2018 | Yes | Severe | Yes | — | — |
| | 18:00:00 | | | | | |
| 1990 | 25 Apr. 2018 | No | — | — | No | — |
| | 18:05:00 | | | | | |
| 1990 | 26 Apr. 2018 | Yes | Moderate | — | Yes | 1 |
| | 22:05:00 | | | | | |
| 1990 | 27 Apr. 2018 | Yes | Moderate | — | Yes | 1 |
| | 18:05:00 | | | | | |
| 1990 | 28 Apr. 2018 | Yes | Severe | — | Yes | 1 |
| | 18:09:00 | | | | | |
| 1990 | 29 Apr. 2018 | No | — | — | No | — |
| | 18:15:00 | | | | | |
| 1990 | 30 Apr. 2018 | Yes | Moderate | — | Yes | 1 |
| | 18:47:00 | | | | | |
| 1990 | 1 May 2018 | No | — | — | No | — |
| | 18:01:00 | | | | | |
| 1990 | 3 May 2018 | No | — | — | No | — |
| | 18:19:00 | | | | | |
| 1990 | 4 May 2018 | Yes | Mild | — | Yes | 1 |
| | 18:45:00 | | | | | |
| 1990 | 6 May 2018 | Yes | Moderate | — | Yes | 1 |
| | 18:00:00 | | | | | |
| 1990 | 7 May 2018 | Yes | Moderate | — | Yes | 1 |
| | 18:00:00 | | | | | |
| 1990 | 8 May 2018 | Yes | Moderate | — | Yes | 1 |
| | 18:00:00 | | | | | |
| 1990 | 9 May 2018 | Yes | Moderate | — | Yes | 1 |
| | 18:05:00 | | | | | |
| 1990 | 10 May 2018 | No | — | — | No | — |
| | 18:02:00 | | | | | |
| 1990 | 13 May 2018 | Yes | Moderate | — | Yes | 1 |
| | 18:01:00 | | | | | |
| 1990 | 14 May 2018 | Yes | Moderate | — | Yes | 1 |
| | 18:04:00 | | | | | |
| 1990 | 15 May 2018 | No | — | — | No | — |
| | 18:03:00 | | | | | |
| 1990 | 16 May 2018 | No | — | — | No | — |
| | 18:08:00 | | | | | |
| 1990 | 17 May 2018 | No | — | — | No | — |
| | 18:01:00 | | | | | |
| 1990 | 18 May 2018 | No | — | — | No | — |
| | 18:15:00 | | | | | |
| 1990 | 19 May 2018 | Yes | Moderate | — | Yes | 1 |
| | 19:50:00 | | | | | |
| 1990 | 20 May 2018 | Yes | Mild | — | Yes | 1 |
| | 18:01:00 | | | | | |
| 1990 | 21 May 2018 | No | — | — | No | — |
| | 18:03:00 | | | | | |
| 1990 | 22 May 2018 | Yes | Moderate | — | Yes | 1 |
| | 18:01:00 | | | | | |
| 1990 | 23 May 2018 | Yes | Severe | — | Yes | 1 |
| | 18:00:00 | | | | | |
| 1990 | 24 May 2018 | Yes | Moderate | — | Yes | 1 |
| | 18:33:00 | | | | | |
| 1990 | 25 May 2018 | Yes | Severe | — | Yes | 1 |
| | 18:02:00 | | | | | |
| 1990 | 26 May 2018 | Yes | Severe | — | Yes | 1 |
| | 18:20:00 | | | | | |
| 1990 | 27 May 2018 | Yes | Severe | — | Yes | 1 |
| | 19:54:00 | | | | | |
| 1990 | 28 May 2018 | Yes | Moderate | — | Yes | 1 |
| | 18:01:00 | | | | | |
| 1990 | 29 May 2018 | Yes | Moderate | — | Yes | 1 |
| | 18:01:00 | | | | | |
| 1990 | 30 May 2018 | Yes | Moderate | — | Yes | 1 |
| | 18:00:00 | | | | | |
| 1990 | 31 May 2018 | No | — | — | No | — |
| | 18:18:00 | | | | | |
| 1990 | 1 Jun. 2018 | No | — | — | No | — |
| | 18:00:00 | | | | | |
| 1990 | 2 Jun. 2018 | No | — | — | No | — |
| | 18:05:00 | | | | | |
| 1990 | 3 Jun. 2018 | Yes | Mild | — | Yes | 1 |
| | 18:07:00 | | | | | |
| 1990 | 4 Jun. 2018 | No | — | — | No | — |
| | 18:00:00 | | | | | |
| 1990 | 5 Jun. 2018 | No | — | — | No | — |
| | 21:47:00 | | | | | |
| 1990 | 6 Jun. 2018 | Yes | Moderate | — | Yes | 1 |
| | 18:03:00 | | | | | |
| 1990 | 7 Jun. 2018 | No | — | — | No | — |
| | 18:05:00 | | | | | |
| 1990 | 8 Jun. 2018 | Yes | Moderate | — | Yes | 1 |
| | 18:00:00 | | | | | |
| 1990 | 10 Jun. 2018 | No | — | — | No | — |
| | 18:00:00 | | | | | |
| 1990 | 13 Jun. 2018 | Yes | Moderate | — | Yes | 1 |
| | 18:04:00 | | | | | |
| 1990 | 14 Jun. 2018 | Yes | Moderate | — | Yes | 1 |
| | 19:16:00 | | | | | |
| 1990 | 15 Jun. 2018 | Yes | Mild | — | Yes | 1 |
| | 18:06:00 | | | | | |
| 1990 | 16 Jun. 2018 | Yes | Mild | — | Yes | 1 |
| | 18:20:00 | | | | | |
| 1990 | 17 Jun. 2018 | Yes | Moderate | — | Yes | 1 |
| | 20:39:00 | | | | | |
| 1990 | 18 Jun. 2018 | Yes | Moderate | — | Yes | 1 |
| | 22:56:00 | | | | | |
| 1990 | 19 Jun. 2018 | Yes | Severe | — | Yes | 1 |
| | 20:39:00 | | | | | |
| 1990 | 20 Jun. 2018 | Yes | Moderate | — | Yes | 1 |
| | 18:46:00 | | | | | |
| 1990 | 21 Jun. 2018 | No | — | — | No | — |
| | 23:27:00 | | | | | |
| 1990 | 26 Jun. 2018 | Yes | Moderate | — | Yes | 1 |
| | 11:20:00 | | | | | |
| 1990 | 26 Jun. 2018 | Yes | Moderate | — | Yes | 1 |
| | 11:21:00 | | | | | |
| 1990 | 26 Jun. 2018 | Yes | Moderate | — | Yes | 1 |
| | 11:22:00 | | | | | |
| 1990 | 26 Jun. 2018 | Yes | Mild | — | Yes | 1 |
| | 18:36:00 | | | | | |
| 1990 | 27 Jun. 2018 | Yes | Mild | — | Yes | 1 |
| | 18:05:00 | | | | | |
| 1990 | 28 Jun. 2018 | No | — | — | No | — |
| | 18:01:00 | | | | | |
| 1990 | 29 Jun. 2018 | No | — | — | No | — |
| | 18:02:00 | | | | | |
| 1990 | 30 Jun. 2018 | Yes | Mild | — | Yes | 1 |
| | 18:00:00 | | | | | |
| 1990 | 1 Jul. 2018 | Yes | Mild | — | Yes | 1 |
| | 18:06:00 | | | | | |
| 1990 | 2 Jul. 2018 | Yes | Moderate | — | Yes | 1 |
| | 18:03:00 | | | | | |
| 1990 | 3 Jul. 2018 | Yes | Moderate | — | Yes | 1 |
| | 18:00:00 | | | | | |
| 1990 | 4 Jul. 2018 | Yes | Moderate | — | Yes | 1 |
| | 18:00:00 | | | | | |
| 1990 | 5 Jul. 2018 | Yes | Moderate | — | Yes | 1 |
| | 18:00:00 | | | | | |
| 1990 | 6 Jul. 2018 | Yes | Moderate | — | Yes | 1 |
| | 18:04:00 | | | | | |
| 1990 | 8 Jul. 2018 | Yes | Moderate | — | Yes | 1 |
| | 21:55:00 | | | | | |
| 1990 | 9 Jul. 2018 | No | — | — | No | — |
| | 18:17:00 | | | | | |
| 1990 | 10 Jul. 2018 | No | — | — | No | — |
| | 20:44:00 | | | | | |
| 1990 | 11 Jul. 2018 | Yes | Moderate | — | Yes | 1 |
| | 18:02:00 | | | | | |
| 1990 | 31 Jul. 2018 | No | — | — | No | — |
| | 12:57:00 | | | | | |

Subject 1990 is a 44 year-old Caucasian female with history of 9-14 migraine attacks per month, who has been receiving erenumab-aooe injections.

Representative observations from subjects that experienced breakthrough are noted below.

Subject A

Subject A is a 36 year old female with a longstanding history of migraine that started at age 17. Her primary migraine type was Migraine with Aura and she reported typically experiencing 11 moderate to severe migraines per month. Her standard medications for the acute treatment of migraine were sumatriptan, Toradol, methadone, Zofran, and Benadryl. She received her first dose of rimegepant on 29 May 2018.

She started treatment with erenumab 140 mg monthly on 3 Aug. 2018. Subsequent to starting treatment with erenumab, she continued to experience migraines. She successfully used rimegepant 75 mg for the acute treatment of these breakthrough migraines. Rimegepant 75 mg was used as needed for acute treatment of migraine on 4 Aug. 2018, 5 Aug. 2018, 8 Aug. 2018, 18 Aug. 2018, and 23 Aug. 2018, 26 Aug. 2018, 27 Aug. 2018, 31 Aug. 2018, 2 Sep. 2018, and 4 Sep. 2018. Acute treatment with rimegepant was effective and she did not require any other acute treatment for migraine on the days that she took the rimegepant. While receiving rimegepant concomitantly with erenumab, she experienced no adverse events.

Subject B

Subject B is a 44 year-old female with a longstanding history of migraine since age 22. She reported her primary migraine type was Migraine without Aura and typically has 8 moderate to severe migraines per month. Her standard medications for migraine were sumatriptan, ibuprofen, and Excedrin Migraine. She received her first dose of rimegepant on 22 Nov. 2017.

She started treatment with erenumab 70 mg monthly on 29 Jun. 2018. Subsequent to starting treatment with erenumab, she continued to experience migraines. She successfully used rimegepant 75 mg for the acute treatment of these breakthrough migraines on 7 Jul. 2018, 8 Jul. 2018, 13 Jul. 2018, 18 Jul. 2018, and 25 Jul. 2018. Acute treatment with rimegepant was effective and she did not require any other acute treatment for migraine on the days that she took the rimegepant. In response to the Preference of Medication question, “How does the study medication compare to the previous medicine(s) your doctor prescribed for your Migraine headache pain?, “the subject responded, “Much better, I prefer this medication.” In response to the Satisfaction with Medication (SM) question, “How satisfied are you with the study medication in treating your migraines?,” the subject responded, “Very satisfied.” While receiving rimegepant concomitantly with erenumab, she experienced no adverse events.

Subject C

Subject C is a 71 year-old female with a long-standing history of migraine for 59 years. She was treated with fremanezumab for six months at a dose of 225 mg per month. She enrolled in BHV-3000-201: Open Label Safety Study in Acute Treatment of Migraine (ClinicalTrials.gov Identifier: NCT 03266588), as described in Example 2. While being treated with fremanezumab, she experienced three migraine breakthroughs that were successfully treated using 75 mg of rimegepant. Successful treatment with rimegepant is defined as clinical and patient reported treatment of migraine without the need for additional acute treatments or rescue meds during that attack.

In another aspect, the underlying treatment may include treatment with a neurotoxic protein, such as Botox.

Representative observations from a subject treated in accordance with the study described in Example 2, that had been treated with abotulinum (Botox®), is described.

Subject D

Subject D is an 18 year-old Caucasian female with history of 9-10 migraine attacks per month since early adolescence. Past medications used to treat her headache have been triptans, calcium channel blockers, metoprolol, topiramate, amitriptyline and oral contraceptives. Additionally, she has been receiving Botox® injections every three months as prophylactic treatment over the prior year. Prior to treatment with Botox®, she would experience >20 migraines per month and after starting prophylactic treatment she would experience 9 migraines per month. Symptom onset with migraine include pain, photophobia, and nausea. If untreated her headaches would become moderate to severe and disabling. When treating her migraines with Zomig® her migraines would decrease in intensity but she would not attain freedom from pain within 24 hrs and would need to take rescue medications including Tylenol and non-steroidal anti-inflammatory medications. Her headaches would persist for days and she would regularly experience rebound headaches. She would have to try to work through persistent pain and experienced muscle pain from triptan use. She also reported a hypersensitivity to sensory stimulation. Her grades “tanked due to migraines” and it had a negative impact on her life.

Subject D enrolled in a long-term safety trial of the oral calcitonin gene peptide antagonist rimegepant which has demonstrated efficacy in two well controlled clinical trials. As part of study participation, Subject D was given a daily supply of rimegepant and allowed to treat her headaches with a single daily dose of rimegepant on an as needed basis. Within one week of entering the study, Subject D experienced the onset of her typical migraine headache. She self-administered rimegepant 75 mg and noted pain relief within a half an hour and her headache did not worsen past mild intensity. Her headache fully resolved within 1 hour. She did not experience persistence of her typical nausea and hypersensitivity to sensations. She was able to function appropriately and engage in her studies after talking treatment, something that Subject D stated she would not typically be able to do when a headache hit. Subject D noted that she remained headache free for the ensuing days and did not require any rescue medications or additional doses of rimegepant. She did not report any adverse effects from treatment.

Subject D also reported that when she receives her typical monthly administration of Botox® the Botox® induces an acute migraine headache that often lasts more than a day; however, she noted that after she received her Botox® she took rimegepant and her Botox® induced headache completely resolved within an hour of taking rimegepant.

The parent of Subject D reported “this is first time in two years that she is back to normal. The stress on the family has consumed lives and can't tell you how much of a relief this is. She has taken 4 or 5 doses of rimegepant this last month and there is a noticeable reduction in the number of headache days that she experienced . . . we believe that there is a preventive effect to taking rimegepant intermittently.”

### Example 5

### Prophylactic Medication Use

In a multicenter, double-blind, phase 3 trial, adults with at least a 1-year history of migraine and two to eight migraine attacks of moderate or severe intensity per month to receive rimegepant orally at a dose of 75 mg or matching placebo for the treatment of a single migraine attack were randomly assigned. The primary end points were freedom from pain and freedom from the most bothersome symptom (other than pain) identified by the patient, both of which were assessed 2 hours after the dose of rimegepant or placebo was administered. Further details of the study can be found at ClinicalTrials.gov number, NCT03237845, the details of which are incorporated by reference herein as is set out in full.

The methods used to measure efficacy end points are described below.

Pain intensity was measured on a four-point ordinal scale (0=none, 1=mild, 2=moderate, 3=severe). Pain freedom was assessed using the number of evaluable patients who reported no pain at two hours postdose. Freedom from the most bothersome symptom was assessed using the number of evaluable patients who reported the absence of their most bothersome symptom at two hours postdose. The most bothersome symptom was measured using a binary scale (0=absent, 1=present). Among secondary efficacy end points, freedom from photophobia, phonophobia, and nausea were assessed by tabulating the number of patients who reported the absence of these symptoms at two hours postdose in the subset of patients who experienced them at baseline. Pain relief at two hours postdose was assessed using the number of evaluable patients reporting moderate or severe pain intensity (two or three on the four-point Likert scale) at baseline and then reporting pain intensity of none or mild (zero or one) at two hours postdose. The probability of requiring rescue medication was assessed using the number of patients who took rescue medication within 24 hours of the administration of study medication. Sustained pain freedom from two to 24 hours and from two to 48 hours postdose was assessed using the number of patients who did not experience any headache pain through the time periods of interest. Sustained pain relief from two to 24 hours and from two to 48 hours postdose was assessed using the number of patients who did not use any rescue medications and did not experience moderate or severe headache pain through the time periods of interest. Pain relapse was assessed using the number of patients who were pain-free at two hours postdose and then had headache pain of any intensity within 48 hours of taking study medication. The proportion of patients able to function normally at two hours postdose was assessed using the number of patients who self-reported normal function on a four-point numeric rating scale (normal function, mild impairment, severe impairment, required bedrest).

Results from the study are shown on Tables 7 to 12.

TABLE 7
Pain Freedom at Two Hours Postdose: Multiple Imputation

| | | | | Risk |
|---|---|---|---|---|
| | | | | Difference |
| | | Rimegepant | Placebo | Rimegepant |
| | Statistic | (N = 537) | (N = 535) | vs Placebo |
| Overall | | | | |
| No Pain | n/N | 105/537 | 64/535 | |
| | Common Risk | 20.2% | 12.4% | 7.82 |
| | ASE | 1.76 | 1.45 | 2.25 |
| | 95% CI | 16.8-23.7 | 9.6-15.3 | 3.4-12.2 |
| | P-value | | | 0.0005 |
| Prophylactic | | | | |
| Medication | | | | |
| Use, Yes | | | | |
| No Pain | n/N | 14/89 | 8/89 | |
| | Stratum Risk | 16.7% | 9.4% | 7.36 |
| | ASE | 4.06 | 3.16 | 5.15 |
| | 95% CI | 8.8-24.7 | 3.2-15.6 | −2.7-17.5 |
| | P-value | | | 0.1530 |
| Prophylactic | | | | |
| Medication | | | | |
| Use, No | | | | |
| No Pain | n/N | 91/448 | 56/446 | |
| | Stratum Risk | 20.9% | 13.0% | 7.91 |
| | ASE | 1.96 | 1.62 | 2.52 |
| | 95% CI | 17.1-24.8 | 9.8-16.2 | 3.0-12.9 |
| | P-value | | | 0.0017 |

Note:
Percentages are based on the number of mITT subjects randomized to each treatment group.
Risk and Risk Difference are calculated using Cochran-Mantel-Haenszel weights, stratified by use of prophylactic medication for the Overall section.
Within each stratum, the risk and risk differences are calculated using a Cochran-Mantel-Haenszel test.
Asymptotic standard errors and 95% CI are presented.
Multiple imputation methods are used to impute missing data at two hours post dose using the copy from reference approach.
The fully conditional specification (FCS) method is used with a generalized logit distribution.
Subjects who used rescue medication at or prior to two hours post dose are imputed as failures.

TABLE 8
Freedom From Most Bothersome Symptom at
Two Hours Postdose: Multiple Imputation

| | | | | Risk |
|---|---|---|---|---|
| | | | | Difference |
| | | Rimegepant | Placebo | Rimegepant |
| | Statistic | (N = 537) | (N = 535) | vs Placebo |
| Overall | | | | |
| Free from MBS | n/N | 202/537 | 135/535 | |
| | Common Risk | 41.2% | 28.4% | 12.82 |
| | ASE | 2.21 | 1.99 | 2.93 |
| | 95% CI | 36.8-45.5 | 24.5-32.3 | 7.1-18.6 |
| | P-value | | | <.0001 |
| Prophylactic | | | | |
| Medication | | | | |
| Use, Yes | | | | |
| Free from MBS | n/N | 31/89 | 13/89 | |
| | Stratum Risk | 40.3% | 17.3% | 23.03 |
| | ASE | 5.58 | 4.17 | 6.93 |
| | 95% CI | 29.4-51.3 | 9.1-25.5 | 9.4-36.6 |
| | P-value | | | 0.0009 |
| Prophylactic | | | | |
| Medication | | | | |
| Use, No | | | | |
| Free from MBS | n/N | 171/448 | 122/446 | |
| | Stratum Risk | 41.4% | 30.6% | 10.79 |
| | ASE | 2.43 | 2.23 | 3.28 |
| | 95% CI | 36.6-46.1 | 26.2-34.9 | 4.4-17.2 |
| | P-value | | | 0.0010 |

Note:
Percentages are based on the number of mITT subjects randomized to each treatment group.
Risk and Risk Difference are calculated using Cochran-Mantel-Haenszel weights, stratified by use of prophylactic medication for the Overall section.
Within each stratum, the risk and risk differences are calculated using a Cochran-Mantel-Haenszel test.
Asymptotic standard errors and 95% CI are presented.
Multiple imputation methods are used to impute missing data at two hours post dose using the copy from reference approach.
The fully conditional specification (FCS) method is used with a generalized logit distribution.
Subjects who used rescue medication at or prior to two hours post dose are imputed as failures.
Subjects who failed to report a most bothersome symptom at study migraine onset, prior to taking study medication, are imputed as failures.

TABLE 9
Pain Freedom at Two Hours Postdose: Varying Success Rate Imputations

| | | Rimegepant | | | |
|---|---|---|---|---|---|
| Placebo | | 0% | 10% | 20% | 30% |
| 0% | Common Risk Difference | 7.59 | 8.15 | 8.52 | 9.08 |
| | Asymptotic 95% CI | 3.3-11.9 | 3.8-12.5 | 4.1-12.9 | 4.7-13.5 |
| | Uncorrected P-value | 0.0006 | 0.0003 | 0.0001 | <.0001 |
| 10% | Common Risk Difference | 7.21 | 7.77 | 8.15 | 8.71 |
| | Asymptotic 95% CI | 2.9-11.6 | 3.4-12.2 | 3.7-12.6 | 4.3-13.1 |
| | Uncorrected P-value | 0.0012 | 0.0005 | 0.0003 | 0.0001 |
| 20% | Common Risk Difference | 6.84 | 7.40 | 7.77 | 8.33 |
| | Asymptotic 95% CI | 2.5-11.2 | 3.0-11.8 | 3.3-12.2 | 3.9-12.8 |
| | Uncorrected P-value | 0.0022 | 0.0010 | 0.0006 | 0.0002 |
| 30% | Common Risk Difference | 6.47 | 7.03 | 7.40 | 7.96 |
| | Asymptotic 95% CI | 2.1-10.9 | 2.6-11.5 | 2.9-11.8 | 3.5-12.4 |
| | Uncorrected P-value | 0.0040 | 0.0019 | 0.0011 | 0.0005 |

Note:
Row percentages represent the hypothetical imputed success rate for Placebo subjects with a missing value at two hours post dose.
Column percentages represent the hypothetical imputed success rate rimegepant subjects with a missing value at two hours post dose.
Risk Difference are calculated using Cochran-Mantel-Haenszel weights, stratified by use of prophylactic medication.
Asymptotic 95% confidence intervals and uncorrected p-values are presented.
Subjects who used rescue medication at or prior to two hours post dose are imputed as failures.

TABLE 10
Freedom From Most Bothersome Symptom at Two Hours
Postdose: Varying Success Rate Imputations

| | | Rimegepant | | | |
|---|---|---|---|---|---|
| Placebo | | 0% | 10% | 20% | 30% |
| 0% | Common Risk Difference | 12.38 | 13.12 | 13.87 | 14.61 |
| | Asymptotic 95% CI | 6.9-17.9 | 7.6-18.6 | 8.4-19.4 | 9.1-20.1 |
| | Uncorrected P-value | <.0001 | <.0001 | <.0001 | <.0001 |
| 10% | Common Risk Difference | 11.82 | 12.56 | 13.31 | 14.05 |
| | Asymptotic 95% CI | 6.3-17.3 | 7.0-18.1 | 7.8-18.8 | 8.5-19.6 |
| | Uncorrected P-value | <.0001 | <.0001 | <.0001 | <.0001 |
| 20% | Common Risk Difference | 11.26 | 12.00 | 12.75 | 13.49 |
| | Asymptotic 95% CI | 5.7-16.8 | 6.5-17.5 | 7.2-18.3 | 7.9-19.1 |
| | Uncorrected P-value | <.0001 | <.0001 | <.0001 | <.0001 |
| 30% | Common Risk Difference | 10.51 | 11.25 | 12.00 | 12.74 |
| | Asymptotic 95% CI | 5.0-16.1 | 5.7-16.8 | 6.4-17.6 | 7.2-18.3 |
| | Uncorrected P-value | 0.0002 | <.0001 | <.0001 | <.0001 |

Note:
Row percentages represent the hypothetical imputed success rate for Placebo subjects with a missing value at two hours post dose.
Column percentages represent the hypothetical imputed success rate rimegepant subjects with a missing value at two hours post dose.
Risk Difference are calculated using Cochran-Mantel-Haenszel weights, stratified by use of prophylactic medication.
Asymptotic 95% confidence intervals and uncorrected p-values are presented.
Subjects who used rescue medication at or prior to two hours post dose are imputed as failures.
Subjects who failed to report a most bothersome symptom at study migraine onset, prior to taking study medication, are imputed as failures.

TABLE 11
Kaplan-Meier Time to First Report of Absence of the Most Bothersome Symptom up to Eight Hours Postdose

| | Rimegepant | | | | | | Placebo | | | | | |
|---|---|---|---|---|---|---|---|---|---|---|---|---|
| | (N = 537) | | | | | | (N = 535) | | | | | |
| Time | N. | N. | N. | | | | N. | N. | N. | N. | | |
| Interval | Risk | Event | Censored | Survival | Lower | Upper | Risk | Event | Censored | Survival | Lower | Upper |
| Minutes | [1] | [2] | [3] | [4] | 95% CI | 95% CI | [1] | [2] | [3] | [4] | 95% CI | 95% CI |
| 0 | 537 | 0 | 0 | 1.00 | | | 535 | 0 | 0 | 1.00 | | |
| 1-30 | 537 | 40 | 3 | 0.93 | 0.90 | 0.94 | 535 | 48 | 7 | 0.91 | 0.88 | 0.93 |
| 31-60 | 494 | 82 | 0 | 0.77 | 0.73 | 0.80 | 480 | 46 | 1 | 0.82 | 0.79 | 0.85 |
| 61-90 | 412 | 53 | 1 | 0.67 | 0.63 | 0.71 | 433 | 40 | 0 | 0.75 | 0.71 | 0.78 |
| 91-120 | 358 | 46 | 2 | 0.59 | 0.54 | 0.63 | 393 | 26 | 0 | 0.70 | 0.66 | 0.73 |
| 121-180 | 310 | 57 | 26 | 0.47 | 0.43 | 0.51 | 367 | 42 | 38 | 0.61 | 0.57 | 0.65 |
| 181-240 | 227 | 36 | 16 | 0.39 | 0.35 | 0.43 | 287 | 28 | 31 | 0.55 | 0.50 | 0.59 |
| 241-360 | 175 | 35 | 14 | 0.31 | 0.27 | 0.35 | 228 | 38 | 26 | 0.45 | 0.40 | 0.49 |
| 361-495 | 126 | 36 | 3 | 0.22 | 0.18 | 0.26 | 164 | 34 | 13 | 0.35 | 0.30 | 0.39 |
| >496 | 87 | 0 | 87 | 0.22 | 0.18 | 0.26 | 117 | 0 | 117 | 0.35 | 0.30 | 0.39 |

CI, confidence interval
[1] The number of subjects at risk is the number of subjects who did not have a score of absent at the start of the specified interval and who had not been censored prior to the interval.
[2] The number of subjects with an event is the number of subjects who reported an absence of their most bothersome symptom for the first time during the specified interval.
[3] The number of subjects censored is the number of subjects who were lost to follow-up during the specified interval. Subjects who did not report absence of their most bothersome symptom by 495 minutes postdose were censored at 496 minutes. Subjects who used rescue medication prior to 8 hours were censored at the time of rescue medication.
[4] Survival estimates were calculated using the Kaplan-Meier Product Limit method.

TABLE 12
Kaplan-Meier Time to Pain Freedom up to Eight Hours Postdose

| | Rimegepant | | | | | | Placebo | | | | | |
|---|---|---|---|---|---|---|---|---|---|---|---|---|
| | (N = 537) | | | | | | (N = 535) | | | | | |
| Time | N. | N. | N. | | | | N. | N. | N. | N. | | |
| Interval | Risk | Event | Censored | Survival | Lower | Upper | Risk | Event | Censored | Survival | Lower | Upper |
| Minutes | [1] | [2] | [3] | [4] | 95% CI | 95% CI | [1] | [2] | [3] | [4] | 95% CI | 95% CI |
| 0 | 537 | 0 | 0 | 1.00 | | | 535 | 0 | 0 | 1.00 | | |
| 1-30 | 537 | 6 | 3 | 0.99 | 0.98 | 0.99 | 535 | 5 | 7 | 0.99 | 0.98 | 1.00 |
| 31-60 | 528 | 36 | 0 | 0.92 | 0.90 | 0.94 | 523 | 19 | 1 | 0.95 | 0.93 | 0.97 |
| 61-90 | 492 | 25 | 1 | 0.87 | 0.84 | 0.90 | 503 | 23 | 0 | 0.91 | 0.88 | 0.93 |
| 91-120 | 466 | 39 | 3 | 0.80 | 0.76 | 0.83 | 480 | 21 | 0 | 0.87 | 0.84 | 0.90 |
| 121-180 | 424 | 64 | 30 | 0.67 | 0.63 | 0.71 | 459 | 35 | 44 | 0.80 | 0.76 | 0.83 |
| 181-240 | 330 | 48 | 22 | 0.57 | 0.53 | 0.61 | 380 | 16 | 36 | 0.76 | 0.72 | 0.80 |
| 241-360 | 260 | 45 | 19 | 0.46 | 0.42 | 0.51 | 328 | 34 | 38 | 0.68 | 0.63 | 0.72 |
| 361-495 | 196 | 51 | 6 | 0.34 | 0.30 | 0.39 | 256 | 53 | 16 | 0.53 | 0.48 | 0.58 |
| >496 | 139 | 0 | 139 | 0.34 | 0.30 | 0.39 | 187 | 0 | 187 | 0.53 | 0.48 | 0.58 |

CI, confidence interval
[1] The number of subjects at risk is the number of subjects who had a pain score of moderate or severe just prior to the start of the specified interval and who had not been censored prior to the interval.
[2] The number of subjects with an event is the number of subjects who had their pain score decrease to no pain for the first time during the specified interval.
[3] The number of subjects censored is the number of subjects who took rescue medication or were lost to follow-up during the specified interval. Subjects who did not have a pain score of none by 495 minutes postdose were censored at 496 minutes.
[4] Survival estimates were calculated using the Kaplan-Meier Product Limit method.

### Example 6

### Treatment Benefit of Small Molecule CGRP Receptor Antagonist Plus Monoclonal Antibody in Migraine Therapy

The following example is summarized below.

Objective

To provide the first clinical report that 2 calcitonin gene-related peptide (CGRP) therapies—a small molecule CGRP receptor antagonist and an anti-CGRP receptor antibody—can be used concomitantly to treat refractory migraine.

Methods

Case reports of 2 patients participating in a long-term safety study of rimegepant 75 mg oral tablets for acute treatment (NCT03266588). After FDA approval of erenumab, both patients started subcutaneous erenumab monthly as allowed per protocol.

Results

Patients were women aged 44 and 36 years with ≥2 decades of self-reported suboptimal response to multiple migraine medications. Patient 1 used rimegepant for 6 months and then started erenumab 70 mg subcutaneous monthly. Despite a response to preventive treatment with erenumab, she experienced substantial relief treating 7 of 7 acute attacks with rimegepant and eliminated regular, frequent use of ibuprofen and a caffeinated analgesic. Patient 2 used rimegepant for 60 days before starting erenumab 140 mg subcutaneously monthly. While on erenumab, 9 of 9 attacks treated with rimegepant responded. She stopped near-daily use of injectable ketorolac and diphenhydramine. While using rimegepant alone or together with erenumab, patients reported no adverse events.

### CONCLUSIONS

Rimegepant 75 mg may be effective for acute treatment during concomitant erenumab preventive administration. The mechanism underlying the benefits of concomitant use of a small molecule CGRP receptor antagonist and an anti-CGRP receptor antibody is unclear and requires further study.

Methods

This example summarizes the cases of 2 patients with migraine who were participating in a long-term safety study of rimegepant 75 mg oral tablets for acute treatment (ClinicalTrials.gov Identifier: NCT03266588). While use of investigational biological agents was prohibited by the protocol, after erenumab approval by the US Food and Drug Administration (May 2018), both patients started subcutaneous monthly preventive therapy. The long-term safety study protocol was approved by independent institutional review boards and/or ethics committees at each trial center. No statistical analyses were performed on the results reported herein; they are based on patient report and investigator observation.

Patient 1

The first patient is a 44 year-old Caucasian woman with a history of migraine without aura since 1995. Prior to enrollment in a trial of rimegepant, she reported an average of 8 attacks with pain of moderate to severe intensity per month during the preceding 3 months. She treated acutely with sumatriptan 100 mg oral tablets or a fixed combination of acetaminophen, acetylsalicylic acid, and caffeine. Ibuprofen was used as needed for dysmenorrhea and migraine.

During a 30-day lead-in phase of the clinical trial, the patient used sumatriptan to treat 10 migraine attacks of moderate to severe pain intensity. After the lead-in phase, she entered the treatment phase of the long-term safety trial and received rimegepant 75 mg as needed, up to once daily, for the acute treatment of migraine. Within 1 week, she discontinued ibuprofen for migraine, and she stopped the caffeine-containing analgesic 5 weeks after entering into treatment with rimegepant 75 mg.

Though her acute attacks responded well to treatment with rimegepant 75 mg, attacks were frequent and after 6 months in the rimegepant long-term safety study, she was started on erenumab 70 mg subcutaneous monthly as a preventive therapy. After starting erenumab, her monthly migraine days (MMDs) declined by 46% over the first 4 weeks from 13 to 7 MMDs of any pain intensity, but she continued to experience migraine attacks. Over the subsequent month, she treated 7 breakthrough migraine attacks that occurred while on erenumab with rimegepant 75 mg oral tablet. Attacks were relieved each time. No other acute migraine medications were needed to resolve the rimegepant-treated attacks. While receiving rimegepant alone, she experienced 1 adverse event of streptococcal pharyngitis that was considered by the investigator to be unrelated to rimegepant. While receiving rimegepant alone or concomitantly with erenumab, she experienced no adverse events related to treatment. At the end-of-study visit, she reported that she was very satisfied with rimegepant and rated it “much better” than previous treatments.

Patient 2

The second patient is a 36 year-old Caucasian woman with a 19-year history of migraine without aura. She reported an average of 11 MHDs with pain of moderate to severe intensity. Her treatment history involved subcutaneous sumatriptan, intranasal zolmitriptan, and oral tablets of rizatriptan, eletriptan, naratriptan, and almotriptan, all of which were suboptimal (e.g., relief took too long, did not last, was inconsistent); she also had a 6-year history of treatment with an implanted occipital nerve stimulator (ONS). At enrollment, her migraine treatments included oral sumatriptan 100 mg, intramuscular (IM) ketorolac tromethamine 30 mg, IM diphenhydramine 100 mg, oral methadone 80 mg, oral ondansetron 8 mg, oral zonisamide 250 mg, and ONS. Prior to enrollment, she stopped using methadone, a prohibited medication for the trial.

During a 30-day run-in to the long-term safety trial, the patient experienced 22 attacks of moderate to severe pain intensity. Upon entry into the treatment phase, she received 30 tablets of rimegepant 75 mg and was instructed to take rimegepant 75 mg up to once per calendar day, as needed, for the acute treatment of migraine attacks of any pain intensity. In the first 30 days of treatment, she used 16 doses of rimegepant; in the second 30 days, she used 11 doses of rimegepant and stopped ondansetron, ketorolac, and diphenhydramine. Because of high headache frequency, she was subsequently started on a monthly dose of erenumab 140 mg. Over the first 30 days after starting erenumab, she experienced 9 attacks, all of which were treated successfully with rimegepant. While using rimegepant alone or together with erenumab, she experienced no adverse events.

Discussion

Rimegepant 75 mg oral tablet and erenumab 70 mg and 140 mg subcutaneous injection have demonstrated efficacy in separate randomized, controlled clinical trials for acute and preventive treatment of migraine, respectively. The response to erenumab in these patients appears typical. However, with histories of long-term polypharmacy with acute medications, both patients were at risk of failing preventive treatment. While the initiation of erenumab reduced MHDs, the onset of treatment with rimegepant enabled the first patient to end 22 years of acute treatment with a caffeine-containing combination analgesic. The second patient eliminated near-daily use of 2 injectable medications: an IM non-steroidal anti-inflammatory drug and an IM anti-nauseant. In the long-term, the reduction of attack frequency and the elimination of regular, frequent use of multiple acute medications are likely to be of substantial clinical import to these patients.

The profile of benefit seen in clinical trials and experiences with rimegepant and erenumab tend to be similar to those described herein and suggests that both compounds will have a meaningful role in the migraine armamentarium. The benefits of their concomitant use may involve additive effects and may be generalizable to other combinations of anti-CGRP agents with distinct molecular targets. Because it is a small case series, this study provides Class IV evidence that combining rimegepant with erenumab may provide effective and safe treatment of patients with a history of refractory migraine.

Quite surprisingly, it has been discovered that the CGRP antagonists of the present invention, e.g., rimegepant, can be used as a prophylactic treatment for migraine.

### Example 7

### Treatment Benefit of Small Molecule CGRP Receptor Antagonist Plus Botulinum Toxin in Migraine Therapy

Certain subjects in the study described in Example 2 were undergoing treatment with botulinum toxin. Records of migraine headache breakthrough are shown in Tables 13-20 below. Subjects that experienced breakthrough, administered rimegepant. Quite surprisingly, subjects that administered rimegepant obtained relief from the migraine headache without the need to take other medications such as triptans or NSAIDS.

TABLE 13

| | | Did you | | Take other | Take study | |
|---|---|---|---|---|---|---|
| | | have a | | med | medication | Number |
| | | migraine | | [Observation | [Treatment | of |
| Subject | Date/Time of Finding | headache? | Severity | phase] | phase] | tablets |
| 1935 | 2018 Mar. 15T22:37:48 | YES | MODERATE | YES | | |
| 1935 | 2018 Mar. 16T19:06:55 | NO | | | | |
| 1935 | 2018 Mar. 17T20:06:25 | NO | | | | |
| 1935 | 2018 Mar. 18T23:18:58 | NO | | | | |
| 1935 | 2018 Mar. 19T19:31:38 | NO | | | | |
| 1935 | 2018 Mar. 20T18:55:59 | NO | | | | |
| 1935 | 2018 Mar. 21T18:59:13 | NO | | | | |
| 1935 | 2018 Mar. 22T20:02:24 | NO | | | | |
| 1935 | 2018 Mar. 23T22:39:29 | NO | | | | |
| 1935 | 2018 Mar. 24T20:36:42 | YES | MILD | YES | | |
| 1935 | 2018 Mar. 25T20:51:44 | YES | MILD | NO | | |
| 1935 | 2018 Mar. 26T20:00:28 | YES | MODERATE | YES | | |
| 1935 | 2018 Mar. 27T18:23:27 | YES | SEVERE | YES | | |
| 1935 | 2018 Mar. 28T23:25:43 | NO | | | | |
| 1935 | 2018 Mar. 29T21:56:39 | NO | | | | |
| 1935 | 2018 Mar. 30T20:48:56 | NO | | | | |
| 1935 | 2018 Mar. 31T21:33:22 | NO | | | | |
| 1935 | 2018 Apr. 1T21:09:16 | NO | | | | |
| 1935 | 2018 Apr. 2T21:01:50 | NO | | | | |
| 1935 | 2018 Apr. 3T22:30:39 | YES | MODERATE | YES | | |
| 1935 | 2018 Apr. 4T23:28:12 | NO | | | | |
| 1935 | 2018 Apr. 5T21:53:32 | YES | MODERATE | YES | | |
| 1935 | 2018 Apr. 6T23:45:42 | NO | | | | |
| 1935 | 2018 Apr. 7T23:09:40 | NO | | | | |
| 1935 | 2018 Apr. 8T20:34:39 | YES | MILD | NO | | |
| 1935 | 2018 Apr. 9T20:45:46 | YES | SEVERE | YES | | |
| 1935 | 2018 Apr. 10T20:06:42 | YES | MILD | NO | | |
| 1935 | 2018 Apr. 11T20:02:31 | YES | MILD | NO | | |
| 1935 | 2018 Apr. 12T20:34:30 | YES | SEVERE | YES | | |
| 1935 | 2018 Apr. 13T20:02:37 | YES | MILD | | NO | |
| 1935 | 2018 Apr. 14T20:16:00 | YES | MILD | | NO | |
| 1935 | 2018 Apr. 15T21:18:44 | NO | | | | |
| 1935 | 2018 Apr. 16T20:32:30 | YES | MILD | | NO | |
| 1935 | 2018 Apr. 17T22:10:25 | YES | MILD | | NO | |
| 1935 | 2018 Apr. 18T19:06:34 | YES | SEVERE | | NO | |
| 1935 | 2018 Apr. 19T22:05:06 | YES | MODERATE | | NO | |
| 1935 | 2018 Apr. 20T20:19:45 | YES | SEVERE | | NO | |
| 1935 | 2018 Apr. 21T20:19:29 | YES | MILD | | NO | |
| 1935 | 2018 Apr. 22T22:01:21 | NO | | | | |
| 1935 | 2018 Apr. 23T20:23:40 | YES | MILD | | NO | |
| 1935 | 2018 Apr. 24T19:31:49 | NO | | | | |
| 1935 | 2018 Apr. 25T20:01:52 | YES | MODERATE | | NO | |
| 1935 | 2018 Apr. 26T20:50:42 | YES | MODERATE | | NO | |
| 1935 | 2018 Apr. 28T18:58:10 | NO | | | | |
| 1935 | 2018 Apr. 29T21:10:48 | YES | MILD | | NO | |
| 1935 | 2018 Apr. 30T21:54:28 | NO | | | | |
| 1935 | 2018 May 1T19:25:32 | YES | MODERATE | | YES | 1 |
| 1935 | 2018 May 2T19:08:05 | NO | | | | |
| 1935 | 2018 May 3T20:05:42 | YES | MILD | | NO | |
| 1935 | 2018 May 5T18:49:21 | NO | | | | |
| 1935 | 2018 May 6T20:08:52 | YES | MILD | | NO | |
| 1935 | 2018 May 7T20:17:57 | YES | MODERATE | | YES | 1 |
| 1935 | 2018 May 8T21:26:17 | NO | | | | |
| 1935 | 2018 May 9T20:23:08 | YES | MODERATE | | YES | 1 |
| 1935 | 2018 May 10T21:45:01 | YES | MODERATE | NO | YES | 1 |
| 1935 | 2018 May 11T20:01:57 | YES | MILD | | YES | 1 |
| 1935 | 2018 May 12T21:58:30 | YES | MODERATE | | YES | 1 |
| 1935 | 2018 May 13T20:49:54 | YES | MODERATE | | YES | 1 |
| 1935 | 2018 May 14T20:03:37 | NO | | | | |
| 1935 | 2018 May 15T20:28:54 | NO | | | NO | |
| 1935 | 2018 May 16T20:25:36 | YES | SEVERE | | YES | 1 |
| 1935 | 2018 May 17T20:06:43 | YES | SEVERE | | YES | 1 |
| 1935 | 2018 May 19T18:35:41 | NO | | | NO | |
| 1935 | 2018 May 20T20:01:43 | NO | | | NO | |
| 1935 | 2018 May 21T22:09:28 | NO | | | NO | |
| 1935 | 2018 May 22T20:50:49 | NO | | | NO | |
| 1935 | 2018 May 23T22:35:56 | YES | SEVERE | | YES | 1 |
| 1935 | 2018 May 24T22:56:29 | YES | MODERATE | | YES | 1 |
| 1935 | 2018 May 25T23:50:17 | YES | SEVERE | | YES | 1 |
| 1935 | 2018 May 26T22:11:40 | NO | | | NO | |
| 1935 | 2018 May 27T23:34:09 | NO | | | NO | |
| 1935 | 2018 May 28T21:18:56 | YES | MODERATE | | YES | 1 |
| 1935 | 2018 May 29T22:36:56 | YES | MODERATE | | YES | 1 |
| 1935 | 2018 May 31T23:37:39 | YES | MODERATE | | YES | 1 |
| 1935 | 2018 Jun. 1T23:21:01 | NO | | | NO | |
| 1935 | 2018 Jun. 2T20:01:54 | NO | | | NO | |
| 1935 | 2018 Jun. 3T20:47:24 | YES | MODERATE | | YES | 1 |
| 1935 | 2018 Jun. 4T20:31:17 | YES | MODERATE | | YES | 1 |
| 1935 | 2018 Jun. 5T20:35:34 | YES | MODERATE | | YES | 1 |
| 1935 | 2018 Jun. 7T20:17:48 | YES | SEVERE | | YES | 1 |
| 1935 | 2018 Jun. 8T19:30:17 | NO | | | NO | |
| 1935 | 2018 Jun. 9T19:22:03 | NO | | | NO | |
| 1935 | 2018 Jun. 10T20:04:23 | YES | MODERATE | | YES | 1 |
| 1935 | 2018 Jun. 11T23:32:00 | NO | | | NO | |
| 1935 | 2018 Jun. 12T20:01:32 | NO | | | NO | |
| 1935 | 2018 Jun. 13T20:17:31 | NO | | | NO | |
| 1935 | 2018 Jun. 14T20:31:59 | NO | | | NO | |
| 1935 | 2018 Jun. 15T18:38:46 | YES | MODERATE | | YES | 1 |
| 1935 | 2018 Jun. 16T19:36:30 | NO | | | NO | |
| 1935 | 2018 Jun. 17T19:29:52 | NO | | | NO | |
| 1935 | 2018 Jun. 18T20:02:05 | NO | | | NO | |
| 1935 | 2018 Jun. 19T19:56:26 | YES | SEVERE | | YES | 1 |
| 1935 | 2018 Jun. 20T19:58:51 | YES | SEVERE | | YES | 1 |
| 1935 | 2018 Jun. 21T20:59:58 | NO | | | NO | |
| 1935 | 2018 Jun. 22T19:31:23 | NO | | | NO | |
| 1935 | 2018 Jun. 23 | YES | SEVERE | | YES | 1 |
| 1935 | 2018 Jun. 24T23:01:14 | NO | | | NO | |
| 1935 | 2018 Jun. 25T22:13:36 | NO | | | NO | |
| 1935 | 2018 Jun. 26T20:32:52 | YES | MODERATE | | YES | 1 |
| 1935 | 2018 Jun. 27T20:16:50 | NO | | | NO | |
| 1935 | 2018 Jun. 28T22:29:09 | NO | | | NO | |
| 1935 | 2018 Jun. 29T22:39:19 | NO | | | NO | |
| 1935 | 2018 Jun. 30T20:04:30 | YES | MODERATE | | YES | 1 |
| 1935 | 2018 Jul. 1T21:53:45 | YES | MODERATE | | YES | 1 |
| 1935 | 2018 Jul. 2T22:21:32 | NO | | | NO | |
| 1935 | 2018 Jul. 3T22:43:41 | NO | | | NO | |
| 1935 | 2018 Jul. 5T20:19:32 | NO | | | NO | |
| 1935 | 2018 Jul. 6T23:41:28 | NO | | | NO | |
| 1935 | 2018 Jul. 7T18:15:08 | NO | | | NO | |
| 1935 | 2018 Jul. 8T20:32:15 | NO | | | NO | |
| 1935 | 2018 Jul. 9T20:35:41 | YES | MODERATE | | YES | 1 |
| 1935 | 2018 Jul. 10T20:46:20 | NO | | | NO | |
| 1935 | 2018 Jul. 11T19:48:04 | YES | MODERATE | | YES | 1 |
| 1935 | 2018 Jul. 12T21:39:54 | NO | | | NO | |
| 1935 | 2018 Jul. 13T22:18:43 | NO | | | NO | |
| 1935 | 2018 Jul. 14T23:57:48 | YES | MODERATE | | YES | 1 |
| 1935 | 2018 Jul. 15T20:47:41 | YES | MODERATE | | YES | 1 |
| 1935 | 2018 Jul. 16T23:09:46 | NO | | | NO | |
| 1935 | 2018 Jul. 17T22:56:26 | YES | MODERATE | | YES | 1 |
| 1935 | 2018 Jul. 18T21:36:35 | YES | MODERATE | | YES | 1 |
| 1935 | 2018 Jul. 19T20:02:47 | YES | MODERATE | | YES | 1 |
| 1935 | 2018 Jul. 20 | YES | SEVERE | | YES | 1 |
| 1935 | 2018 Jul. 21T20:01:09 | YES | MODERATE | | YES | 1 |
| 1935 | 2018 Jul. 22T22:19:42 | NO | | | NO | |
| 1935 | 2018 Jul. 23T22:18:22 | YES | MODERATE | | YES | 1 |
| 1935 | 2018 Jul. 24T23:44:15 | YES | MODERATE | | YES | 1 |
| 1935 | 2018 Jul. 25T22:13:13 | NO | | | NO | |
| 1935 | 2018 Jul. 26T21:05:46 | YES | MODERATE | | YES | 1 |
| 1935 | 2018 Jul. 27T21:47:12 | NO | | | NO | |
| 1935 | 2018 Jul. 28T21:43:26 | YES | MODERATE | | YES | 1 |
| 1935 | 2018 Jul. 29T20:34:31 | NO | | | NO | |
| 1935 | 2018 Jul. 30T21:28:22 | NO | | | NO | |
| 1935 | 2018 Jul. 31T20:17:42 | NO | | | NO | |
| 1935 | 2018 Aug. 1T22:52:31 | NO | | | NO | |
| 1935 | 2018 Aug. 2T21:24:00 | YES | MODERATE | | YES | 1 |
| 1935 | 2018 Aug. 3T21:04:52 | NO | | | NO | |
| 1935 | 2018 Aug. 4T19:58:39 | YES | MODERATE | | YES | 1 |
| 1935 | 2018 Aug. 5T20:02:28 | YES | SEVERE | | YES | 1 |
| 1935 | 2018 Aug. 6T20:01:00 | YES | MODERATE | | YES | 1 |
| 1935 | 2018 Aug. 7T21:50:17 | NO | | | NO | |
| 1935 | 2018 Aug. 8T22:03:49 | NO | | | NO | |
| 1935 | 2018 Aug. 9T20:30:58 | NO | | | NO | |
| 1935 | 2018 Aug. 10T20:04:59 | YES | SEVERE | | YES | 1 |
| 1935 | 2018 Aug. 11T23:00:26 | YES | MODERATE | | YES | 1 |
| 1935 | 2018 Aug. 12T20:00:58 | NO | | | NO | |
| 1935 | 2018 Aug. 13T21:39:35 | YES | MODERATE | | YES | 1 |
| 1935 | 2018 Aug. 14T23:39:32 | NO | | | NO | |
| 1935 | 2018 Aug. 15T22:33:33 | NO | | | NO | |
| 1935 | 2018 Aug. 16T22:11:26 | YES | SEVERE | | YES | 1 |
| 1935 | 2018 Aug. 17T23:14:12 | YES | SEVERE | | YES | 1 |
| 1935 | 2018 Aug. 18T23:42:38 | NO | | | NO | |
| 1935 | 2018 Aug. 19T23:21:40 | YES | MODERATE | | YES | 1 |
| 1935 | 2018 Aug. 20T20:33:25 | YES | MODERATE | | YES | 1 |
| 1935 | 2018 Aug. 21T21:57:06 | NO | | | NO | |
| 1935 | 2018 Aug. 22T22:02:30 | NO | | | NO | |
| 1935 | 2018 Aug. 23T22:10:08 | YES | SEVERE | | YES | 1 |
| 1935 | 2018 Aug. 24T20:32:49 | YES | SEVERE | | YES | 1 |
| 1935 | 2018 Aug. 25T21:38:53 | NO | | | NO | |
| 1935 | 2018 Aug. 26T22:09:25 | YES | MODERATE | | YES | 1 |
| 1935 | 2018 Aug. 27 | YES | MODERATE | | YES | 1 |
| 1935 | 2018 Aug. 28T20:12:12 | NO | | | NO | |
| 1935 | 2018 Aug. 29T20:54:12 | YES | SEVERE | | YES | 1 |
| 1935 | 2018 Aug. 30T21:01:42 | NO | | | NO | |
| 1935 | 2018 Aug. 31T20:45:47 | NO | | | NO | |
| 1935 | 2018 Sep. 1T20:54:46 | NO | | | NO | |
| 1935 | 2018 Sep. 2T20:15:08 | NO | | | NO | |
| 1935 | 2018 Sep. 3T20:20:30 | NO | | | NO | |
| 1935 | 2018 Sep. 4T20:57:59 | YES | MODERATE | | YES | 1 |
| 1935 | 2018 Sep. 5T23:33:00 | NO | | | NO | |
| 1935 | 2018 Sep. 6 | YES | MODERATE | | YES | 1 |
| 1935 | 2018 Sep. 7T19:17:11 | YES | MODERATE | | YES | 1 |
| 1935 | 2018 Sep. 8T22:01:26 | YES | MODERATE | | YES | 1 |
| 1935 | 2018 Sep. 9T20:00:31 | NO | | | NO | |
| 1935 | 2018 Sep. 10T23:40:54 | NO | | | NO | |
| 1935 | 2018 Sep. 11T19:09:23 | YES | MODERATE | | YES | 1 |
| 1935 | 2018 Sep. 12T20:01:43 | NO | | | NO | |
| 1935 | 2018 Sep. 13T20:01:15 | NO | | | NO | |
| 1935 | 2018 Sep. 14T22:53:03 | YES | MODERATE | | YES | 1 |
| 1935 | 2018 Sep. 15T20:01:19 | NO | | | NO | |
| 1935 | 2018 Sep. 16T21:23:01 | NO | | | NO | |
| 1935 | 2018 Sep. 17T20:04:55 | YES | MODERATE | | YES | 1 |
| 1935 | 2018 Sep. 18T20:43:54 | YES | MODERATE | | YES | 1 |
| 1935 | 2018 Sep. 19T23:01:41 | NO | | | NO | |
| 1935 | 2018 Sep. 20T20:04:05 | NO | | | NO | |
| 1935 | 2018 Sep. 21T22:17:04 | YES | MODERATE | | YES | 1 |
| 1935 | 2018 Sep. 22T22:42:14 | NO | | | NO | |
| 1935 | 2018 Sep. 23T21:52:52 | YES | MODERATE | | YES | 1 |
| 1935 | 2018 Sep. 24T22:09:35 | NO | | | NO | |
| 1935 | 2018 Sep. 25T20:21:39 | YES | MODERATE | | YES | 1 |
| 1935 | 2018 Sep. 26T20:19:01 | YES | MODERATE | | YES | 1 |
| 1935 | 2018 Sep. 27T23:02:30 | NO | | | NO | |
| 1935 | 2018 Sep. 28T23:46:04 | NO | | | NO | |
| 1935 | 2018 Sep. 29T20:23:12 | NO | | | NO | |
| 1935 | 2018 Sep. 30T23:17:06 | NO | | | NO | |
| 1935 | 2018 Oct. 1T21:56:59 | NO | | | NO | |
| 1935 | 2018 Oct. 2T21:21:12 | YES | SEVERE | | YES | 1 |
| 1935 | 2018 Oct. 3T19:51:28 | NO | | | NO | |
| 1935 | 2018 Oct. 4T20:51:10 | YES | MODERATE | | YES | 1 |
| 1935 | 2018 Oct. 5T22:56:06 | NO | | | NO | |
| 1935 | 2018 Oct. 6T20:15:52 | NO | | | NO | |
| 1935 | 2018 Oct. 7T21:47:07 | NO | | | NO | |
| 1935 | 2018 Oct. 8T20:03:04 | YES | MODERATE | | YES | 1 |
| 1935 | 2018 Oct. 9T20:05:05 | YES | MODERATE | | YES | 1 |
| 1935 | 2018 Oct. 10T20:05:58 | YES | MODERATE | | YES | 1 |
| 1935 | 2018 Oct. 11T20:01:07 | NO | | | NO | |
| 1935 | 2018 Oct. 12T22:29:16 | YES | MODERATE | | YES | 1 |
| 1935 | 2018 Oct. 13T20:04:06 | NO | | | NO | |
| 1935 | 2018 Oct. 14T22:00:13 | NO | | | NO | |
| 1935 | 2018 Oct. 15T21:53:20 | YES | SEVERE | | YES | 1 |
| 1935 | 2018 Oct. 16T22:28:21 | YES | SEVERE | | YES | 1 |
| 1935 | 2018 Oct. 17T22:10:56 | NO | | | NO | |
| 1935 | 2018 Oct. 18T19:30:03 | NO | | | NO | |
| 1935 | 2018 Oct. 19T23:43:59 | YES | MODERATE | | YES | 1 |
| 1935 | 2018 Oct. 20T20:05:00 | YES | MODERATE | | YES | 1 |
| 1935 | 2018 Oct. 21T22:04:48 | NO | | | NO | |
| 1935 | 2018 Oct. 22T22:09:39 | YES | SEVERE | | YES | 1 |
| 1935 | 2018 Oct. 23 | YES | MODERATE | | YES | 1 |
| 1935 | 2018 Oct. 24T23:06:02 | YES | SEVERE | | YES | 1 |
| 1935 | 2018 Oct. 25T19:43:36 | NO | | | NO | |
| 1935 | 2018 Oct. 26T21:46:35 | YES | MODERATE | | YES | 1 |
| 1935 | 2018 Oct. 27T20:30:17 | NO | | | NO | |
| 1935 | 2018 Oct. 28T20:57:33 | NO | | | NO | |
| 1935 | 2018 Oct. 29T22:31:59 | NO | | | NO | |
| 1935 | 2018 Oct. 30T20:45:47 | NO | | | NO | |
| 1935 | 2018 Oct. 31T20:29:30 | NO | | | NO | |
| 1935 | 2018 Nov. 1T20:28:17 | YES | MODERATE | | YES | 1 |
| 1935 | 2018 Nov. 2T21:43:10 | YES | MODERATE | | YES | 1 |
| 1935 | 2018 Nov. 3T20:00:29 | NO | | | NO | |
| 1935 | 2018 Nov. 4T20:00:44 | YES | MODERATE | | YES | 1 |
| 1935 | 2018 Nov. 5T20:15:23 | YES | MODERATE | | YES | 1 |
| 1935 | 2018 Nov. 6T20:30:41 | NO | | | NO | |
| 1935 | 2018 Nov. 7T20:45:54 | NO | | | NO | |
| 1935 | 2018 Nov. 8T19:49:38 | NO | | | NO | |
| 1935 | 2018 Nov. 9T23:13:55 | YES | MODERATE | | YES | 1 |
| 1935 | 2018 Nov. 10T19:37:05 | YES | MODERATE | | YES | 1 |
| 1935 | 2018 Nov. 11T20:04:13 | NO | | | NO | |
| 1935 | 2018 Nov. 12T20:01:42 | YES | MODERATE | | YES | 1 |
| 1935 | 2018 Nov. 13T20:16:51 | NO | | | NO | |
| 1935 | 2018 Nov. 14T20:00:46 | NO | | | NO | |
| 1935 | 2018 Nov. 15T21:29:46 | YES | MODERATE | | YES | 1 |
| 1935 | 2018 Nov. 16T20:41:53 | YES | SEVERE | | YES | 1 |
| 1935 | 2018 Nov. 17T20:07:10 | NO | | | NO | |
| 1935 | 2018 Nov. 18T20:49:50 | NO | | | NO | |
| 1935 | 2018 Nov. 19T20:46:18 | YES | MODERATE | | YES | 1 |
| 1935 | 2018 Nov. 20T20:02:36 | YES | MODERATE | | YES | 1 |
| 1935 | 2018 Nov. 21T20:00:44 | YES | MODERATE | | YES | 1 |
| 1935 | 2018 Nov. 22T22:29:26 | YES | MODERATE | | YES | 1 |
| 1935 | 2018 Nov. 23T20:01:44 | NO | | | NO | |
| 1935 | 2018 Nov. 24 | YES | MODERATE | | YES | 1 |
| 1935 | 2018 Nov. 25T22:38:34 | NO | | | NO | |
| 1935 | 2018 Nov. 26T20:18:17 | YES | MODERATE | | YES | 1 |
| 1935 | 2018 Nov. 27T20:33:59 | YES | MODERATE | | YES | 1 |
| 1935 | 2018 Nov. 28T20:14:04 | YES | MODERATE | | YES | 1 |
| 1935 | 2018 Nov. 29T20:42:36 | NO | | | NO | |
| 1935 | 2018 Nov. 30T23:15:36 | NO | | | NO | |
| 1935 | 2018 Dec. 1T19:27:15 | NO | | | NO | |
| 1935 | 2018 Dec. 2T21:47:42 | NO | | | NO | |
| 1935 | 2018 Dec. 3T20:02:22 | YES | MODERATE | | YES | 1 |
| 1935 | 2018 Dec. 4T22:12:38 | YES | SEVERE | | YES | 1 |
| 1935 | 2018 Dec. 5T21:48:03 | YES | SEVERE | | YES | 1 |
| 1935 | 2018 Dec. 6T20:32:59 | NO | | | NO | |
| 1935 | 2018 Dec. 7T21:39:46 | YES | MODERATE | | YES | 1 |
| 1935 | 2018 Dec. 8T19:50:15 | NO | | | NO | |
| 1935 | 2018 Dec. 9T22:18:51 | YES | MODERATE | | YES | 1 |
| 1935 | 2018 Dec. 10T19:35:19 | NO | | | NO | |
| 1935 | 2018 Dec. 11T21:48:39 | YES | MODERATE | | YES | 1 |
| 1935 | 2018 Dec. 12T22:36:15 | NO | | | NO | |
| 1935 | 2018 Dec. 13T20:47:25 | NO | | | NO | |
| 1935 | 2018 Dec. 14T23:34:08 | YES | MODERATE | | YES | 1 |
| 1935 | 2018 Dec. 15T20:23:40 | NO | | | NO | |
| 1935 | 2018 Dec. 16T20:32:50 | NO | | | NO | |
| 1935 | 2018 Dec. 17T22:47:48 | YES | MODERATE | | YES | 1 |
| 1935 | 2018 Dec. 18T21:26:13 | YES | MODERATE | | YES | 1 |
| 1935 | 2018 Dec. 19T20:21:19 | NO | | | NO | |
| 1935 | 2018 Dec. 20T20:50:36 | YES | MODERATE | | YES | 1 |
| 1935 | 2018 Dec. 21T21:11:56 | NO | | | NO | |
| 1935 | 2018 Dec. 22T21:04:58 | NO | | | NO | |
| 1935 | 2018 Dec. 23T20:46:03 | YES | MODERATE | | YES | 1 |
| 1935 | 2018 Dec. 24T20:06:02 | YES | MODERATE | | YES | 1 |
| 1935 | 2018 Dec. 25T22:58:30 | NO | | | NO | |
| 1935 | 2018 Dec. 26T20:05:53 | NO | | | NO | |
| 1935 | 2018 Dec. 27T22:57:02 | NO | | | NO | |
| 1935 | 2018 Dec. 28T20:08:12 | YES | MODERATE | | YES | 1 |
| 1935 | 2018 Dec. 29T20:08:00 | YES | MODERATE | | YES | 1 |
| 1935 | 2018 Dec. 30T22:14:37 | NO | | | NO | |
| 1935 | 2018 Dec. 31T19:44:05 | NO | | | NO | |
| 1935 | 2019 Jan. 1T21:56:26 | NO | | | NO | |
| 1935 | 2019 Jan. 2T22:21:52 | NO | | | NO | |
| 1935 | 2019 Jan. 3T20:55:10 | NO | | | NO | |
| 1935 | 2019 Jan. 4T20:15:53 | NO | | | NO | |
| 1935 | 2019 Jan. 5T23:31:12 | NO | | | NO | |
| 1935 | 2019 Jan. 6T22:43:35 | NO | | | NO | |
| 1935 | 2019 Jan. 7T23:09:31 | NO | | | NO | |
| 1935 | 2019 Jan. 8T23:22:38 | NO | | | NO | |
| 1935 | 2019 Jan. 9T22:43:49 | NO | | | NO | |
| 1935 | 2019 Jan. 10T19:58:17 | NO | | | NO | |
| 1935 | 2019 Jan. 11T20:04:30 | NO | | | NO | |
| 1935 | 2019 Jan. 12T20:00:16 | NO | | | NO | |
| 1935 | 2019 Jan. 13T21:02:19 | YES | MODERATE | | YES | 1 |
| 1935 | 2019 Jan. 14T21:34:25 | YES | MODERATE | | YES | 1 |
| 1935 | 2019 Jan. 15T19:59:27 | YES | MODERATE | | YES | 1 |
| 1935 | 2019 Jan. 16T20:19:40 | NO | | | NO | |
| 1935 | 2019 Jan. 17T18:31:20 | NO | | | NO | |
| 1935 | 2019 Jan. 18T23:01:11 | NO | | | NO | |
| 1935 | 2019 Jan. 19T20:16:54 | NO | | | NO | |
| 1935 | 2019 Jan. 20T23:32:46 | NO | | | NO | |
| 1935 | 2019 Jan. 21T20:51:15 | YES | MODERATE | | YES | 1 |
| 1935 | 2019 Jan. 22T20:12:57 | YES | MODERATE | | YES | 1 |
| 1935 | 2019 Jan. 23T20:15:35 | YES | MODERATE | | YES | 1 |
| 1935 | 2019 Jan. 24T20:01:35 | NO | | | NO | |
| 1935 | 2019 Jan. 25T23:16:36 | YES | MODERATE | | YES | 1 |
| 1935 | 2019 Jan. 26T20:31:41 | NO | | | NO | |
| 1935 | 2019 Jan. 27T20:09:52 | YES | MODERATE | | YES | 1 |
| 1935 | 2019 Jan. 28T20:30:50 | YES | MODERATE | | YES | 1 |
| 1935 | 2019 Jan. 29T20:05:26 | YES | MODERATE | | YES | 1 |
| 1935 | 2019 Jan. 30T20:31:02 | YES | MODERATE | | YES | 1 |
| 1935 | 2019 Jan. 31T20:16:12 | NO | | | NO | |
| 1935 | 2019 Feb. 1T20:33:23 | YES | MODERATE | | YES | 1 |
| 1935 | 2019 Feb. 2T21:20:35 | NO | | | NO | |
| 1935 | 2019 Feb. 3T19:47:42 | NO | | | NO | |
| 1935 | 2019 Feb. 4T20:19:45 | NO | | | NO | |
| 1935 | 2019 Feb. 5T20:01:45 | YES | SEVERE | | YES | 1 |
| 1935 | 2019 Feb. 6T20:59:01 | NO | | | NO | |
| 1935 | 2019 Feb. 7T22:49:07 | YES | MODERATE | | YES | 1 |
| 1935 | 2019 Feb. 8T20:32:59 | YES | SEVERE | | YES | 1 |
| 1935 | 2019 Feb. 9T20:15:30 | YES | MODERATE | | YES | 1 |
| 1935 | 2019 Feb. 10T20:36:22 | YES | MODERATE | | YES | 1 |
| 1935 | 2019 Feb. 11T20:33:36 | YES | MODERATE | | YES | 1 |
| 1935 | 2019 Feb. 12T19:41:36 | YES | MODERATE | | YES | 1 |
| 1935 | 2019 Feb. 13T20:02:18 | NO | | | NO | |
| 1935 | 2019 Feb. 14T20:19:38 | NO | | | NO | |
| 1935 | 2019 Feb. 15T22:35:38 | YES | MODERATE | | YES | 1 |
| 1935 | 2019 Feb. 16T20:54:20 | YES | MODERATE | | YES | 1 |
| 1935 | 2019 Feb. 17T20:04:26 | NO | | | NO | |
| 1935 | 2019 Feb. 18T20:47:50 | YES | MODERATE | | YES | 1 |
| 1935 | 2019 Feb. 19T20:02:01 | YES | MODERATE | | YES | 1 |
| 1935 | 2019 Feb. 20T20:01:36 | YES | MODERATE | | YES | 1 |
| 1935 | 2019 Feb. 21T20:31:08 | YES | MODERATE | | YES | 1 |
| 1935 | 2019 Feb. 22T20:00:49 | YES | MODERATE | | YES | 1 |
| 1935 | 2019 Feb. 23T20:01:54 | NO | | | NO | |
| 1935 | 2019 Feb. 25T21:01:16 | YES | MODERATE | | YES | 1 |
| 1935 | 2019 Feb. 26T20:16:40 | YES | MODERATE | | YES | 1 |
| 1935 | 2019 Feb. 27T22:13:16 | YES | MODERATE | | YES | 1 |
| 1935 | 2019 Feb. 28T20:24:45 | YES | MODERATE | | YES | 1 |
| 1935 | 2019 Mar. 1T20:03:08 | YES | MODERATE | | YES | 1 |
| 1935 | 2019 Mar. 2T20:02:35 | YES | SEVERE | | YES | 1 |
| 1935 | 2019 Mar. 4T20:39:13 | YES | MODERATE | | YES | 1 |
| 1935 | 2019 Mar. 5T20:05:47 | YES | MODERATE | | YES | 1 |
| 1935 | 2019 Mar. 6T20:01:41 | NO | | | NO | |
| 1935 | 2019 Mar. 7T23:43:37 | YES | MODERATE | | YES | 1 |
| 1935 | 2019 Mar. 8T19:38:03 | YES | MODERATE | | YES | 1 |
| 1935 | 2019 Mar. 9T20:37:09 | YES | MODERATE | | YES | 1 |
| 1935 | 2019 Mar. 10T20:32:25 | NO | | | NO | |
| 1935 | 2019 Mar. 11T21:41:06 | YES | MODERATE | | YES | 1 |
| 1935 | 2019 Mar. 12T20:18:32 | NO | | | NO | |
| 1935 | 2019 Mar. 13T20:00:28 | YES | MODERATE | | YES | 1 |
| 1935 | 2019 Mar. 14T20:51:03 | NO | | | NO | |
| 1935 | 2019 Mar. 15T20:32:40 | YES | MODERATE | | YES | 1 |
| 1935 | 2019 Mar. 16T22:20:53 | NO | | | NO | |
| 1935 | 2019 Mar. 17T20:45:34 | YES | MODERATE | | YES | 1 |
| 1935 | 2019 Mar. 18T20:46:59 | YES | MODERATE | | YES | 1 |
| 1935 | 2019 Mar. 19T20:18:46 | YES | MODERATE | | YES | 1 |
| 1935 | 2019 Mar. 20T20:02:24 | YES | MODERATE | | YES | 1 |
| 1935 | 2019 Mar. 21T20:18:43 | YES | MODERATE | | YES | 1 |
| 1935 | 2019 Mar. 22T22:34:01 | YES | MODERATE | | YES | 1 |
| 1935 | 2019 Mar. 23T20:00:50 | YES | MODERATE | | YES | 1 |
| 1935 | 2019 Mar. 24T23:01:15 | YES | MODERATE | | YES | 1 |
| 1935 | 2019 Mar. 25T20:04:26 | YES | MODERATE | | YES | 1 |
| 1935 | 2019 Mar. 26T20:05:17 | YES | MODERATE | | YES | 1 |
| 1935 | 2019 Mar. 27T22:07:18 | NO | | | NO | |
| 1935 | 2019 Mar. 28T20:43:44 | YES | MODERATE | | YES | 1 |
| 1935 | 2019 Mar. 30T20:30:56 | YES | MODERATE | | YES | 1 |
| 1935 | 2019 Mar. 31T20:00:22 | NO | | | NO | |
| 1935 | 2019 Apr. 1T20:45:44 | YES | MODERATE | | YES | 1 |
| 1935 | 2019 Apr. 2T19:06:35 | YES | MODERATE | | YES | 1 |
| 1935 | 2019 Apr. 3T20:29:20 | NO | | | NO | |
| 1935 | 2019 Apr. 4T20:44:20 | NO | | | NO | |
| 1935 | 2019 Apr. 5T20:48:11 | NO | | | NO | |
| 1935 | 2019 Apr. 6T20:05:36 | YES | SEVERE | | YES | 1 |
| 1935 | 2019 Apr. 7T20:03:09 | NO | | | NO | |
| 1935 | 2019 Apr. 8T20:02:32 | YES | MODERATE | | YES | 1 |
| 1935 | 2019 Apr. 9T20:01:37 | YES | MODERATE | | YES | 1 |
| 1935 | 2019 Apr. 10T20:11:58 | NO | | | NO | |
| 1935 | 2019 Apr. 11T20:49:59 | YES | MODERATE | | YES | 1 |
| 1935 | 2019 Apr. 12T20:00:45 | YES | MODERATE | | YES | 1 |
| 1935 | 2019 Apr. 13T20:49:40 | YES | MODERATE | | YES | 1 |
| 1935 | 2019 Apr. 14T20:09:42 | YES | MODERATE | | YES | 1 |
| 1935 | 2019 Apr. 16T20:48:34 | YES | SEVERE | | YES | 1 |
| 1935 | 2019 Apr. 17T20:00:39 | YES | MODERATE | | YES | 1 |
| 1935 | 2019 Apr. 18T15:24:55 | YES | MODERATE | | YES | 1 |

Subject 1935 is a 31 year-old Caucasian female with history of 9-14 migraine attacks (without aura) per month since the age of 25. Past medications used to treat her headache have been rizatriptan and naproxen. Additionally, she has been receiving Botox® injections every three months from 16 Jan. 2017.

TABLE 14

| | | Did you | | Take other | Take study | |
|---|---|---|---|---|---|---|
| | | have a | | med | medication | Number |
| | | migraine | | [Observation | [Treatment | of |
| Subject | Date/Time of Finding | headache? | Severity | phase] | phase] | tablets |
| 2053 | 2018 Apr. 5T18:31:49 | NO | | | | |
| 2053 | 2018 Apr. 6T18:06:31 | NO | | | | |
| 2053 | 2018 Apr. 7T18:02:25 | NO | | | | |
| 2053 | 2018 Apr. 8T18:01:41 | NO | | | | |
| 2053 | 2018 Apr. 9T20:24:14 | YES | MODERATE | YES | | |
| 2053 | 2018 Apr. 10T18:01:55 | YES | MILD | NO | | |
| 2053 | 2018 Apr. 11T18:03:56 | NO | | | | |
| 2053 | 2018 Apr. 12T18:01:51 | NO | | | | |
| 2053 | 2018 Apr. 13T18:04:44 | NO | | | | |
| 2053 | 2018 Apr. 14T18:00:55 | NO | | | | |
| 2053 | 2018 Apr. 15T18:01:12 | NO | | | | |
| 2053 | 2018 Apr. 16T18:01:11 | YES | MODERATE | YES | | |
| 2053 | 2018 Apr. 17T18:02:22 | YES | MILD | NO | | |
| 2053 | 2018 Apr. 18T18:00:43 | NO | | | | |
| 2053 | 2018 Apr. 19T18:00:37 | NO | | | | |
| 2053 | 2018 Apr. 20T18:01:31 | YES | MILD | NO | | |
| 2053 | 2018 Apr. 21T18:03:11 | NO | | | | |
| 2053 | 2018 Apr. 22T18:01:36 | YES | SEVERE | YES | | |
| 2053 | 2018 Apr. 23T18:08:25 | YES | MILD | NO | | |
| 2053 | 2018 Apr. 24T18:05:05 | YES | MILD | NO | | |
| 2053 | 2018 Apr. 25T18:01:10 | NO | | | | |
| 2053 | 2018 Apr. 26T18:00:44 | YES | MILD | NO | | |
| 2053 | 2018 Apr. 28T18:00:40 | NO | | | | |
| 2053 | 2018 Apr. 29T19:22:20 | NO | | | | |
| 2053 | 2018 Apr. 30T18:00:50 | YES | SEVERE | YES | | |
| 2053 | 2018 May 1T18:02:56 | NO | | | | |
| 2053 | 2018 May 2T18:00:44 | NO | | | | |
| 2053 | 2018 May 3T18:30:50 | YES | MILD | NO | | |
| 2053 | 2018 May 4T19:45:37 | YES | SEVERE | YES | | |
| 2053 | 2018 May 5T18:32:22 | NO | | | NO | |
| 2053 | 2018 May 6T18:00:53 | NO | | | NO | |
| 2053 | 2018 May 7T18:00:38 | NO | | | NO | |
| 2053 | 2018 May 8T18:17:50 | YES | SEVERE | | YES | 1 |
| 2053 | 2018 May 9T18:16:32 | NO | | | NO | |
| 2053 | 2018 May 10T19:46:47 | NO | | | NO | |
| 2053 | 2018 May 11T20:41:36 | NO | | | NO | |
| 2053 | 2018 May 13T18:07:17 | NO | | | NO | |
| 2053 | 2018 May 14T18:00:59 | YES | SEVERE | | YES | 1 |
| 2053 | 2018 May 15T18:01:42 | YES | MILD | | NO | |
| 2053 | 2018 May 16T18:17:36 | NO | | | NO | |
| 2053 | 2018 May 17T18:03:41 | NO | | | NO | |
| 2053 | 2018 May 18T18:16:51 | YES | SEVERE | | YES | 1 |
| 2053 | 2018 May 19T18:18:29 | NO | | | NO | |
| 2053 | 2018 May 20T18:00:28 | NO | | | NO | |
| 2053 | 2018 May 21T18:33:51 | YES | SEVERE | | YES | 1 |
| 2053 | 2018 May 22T18:02:25 | NO | | | NO | |
| 2053 | 2018 May 23T18:38:55 | NO | | | NO | |
| 2053 | 2018 May 24T18:06:51 | NO | | | NO | |
| 2053 | 2018 May 25T18:01:55 | NO | | | NO | |
| 2053 | 2018 May 26T18:00:32 | NO | | | NO | |
| 2053 | 2018 May 27T21:22:51 | NO | | | NO | |
| 2053 | 2018 May 28T18:02:47 | NO | | | NO | |
| 2053 | 2018 May 29T18:01:05 | YES | SEVERE | | YES | 1 |
| 2053 | 2018 May 30T18:01:36 | NO | | | NO | |
| 2053 | 2018 May 31T18:18:00 | NO | | | NO | |
| 2053 | 2018 Jun. 1T18:04:57 | NO | | | NO | |
| 2053 | 2018 Jun. 2T18:01:38 | NO | | | NO | |
| 2053 | 2018 Jun. 3T18:00:29 | YES | SEVERE | | YES | 1 |
| 2053 | 2018 Jun. 4T18:04:39 | YES | SEVERE | | YES | 1 |
| 2053 | 2018 Jun. 5T18:01:38 | NO | | | NO | |
| 2053 | 2018 Jun. 6T18:00:45 | YES | MILD | | NO | |
| 2053 | 2018 Jun. 7T18:00:37 | NO | | | NO | |
| 2053 | 2018 Jun. 8T18:13:20 | NO | | | NO | |
| 2053 | 2018 Jun. 9T18:00:37 | NO | | | NO | |
| 2053 | 2018 Jun. 10T18:06:03 | NO | | | NO | |
| 2053 | 2018 Jun. 11T18:10:45 | YES | SEVERE | | YES | 1 |
| 2053 | 2018 Jun. 12T18:05:18 | YES | MILD | | NO | |
| 2053 | 2018 Jun. 13T18:05:02 | NO | | | NO | |
| 2053 | 2018 Jun. 14T18:22:06 | NO | | | NO | |
| 2053 | 2018 Jun. 15T18:00:46 | NO | | | NO | |
| 2053 | 2018 Jun. 16T18:15:47 | NO | | | NO | |
| 2053 | 2018 Jun. 17T18:01:05 | YES | SEVERE | | YES | 1 |
| 2053 | 2018 Jun. 18T18:00:43 | YES | SEVERE | | YES | 1 |
| 2053 | 2018 Jun. 19T18:00:44 | NO | | | NO | |
| 2053 | 2018 Jun. 20T18:15:46 | NO | | | NO | |
| 2053 | 2018 Jun. 21T18:02:05 | NO | | | NO | |
| 2053 | 2018 Jun. 22T18:04:15 | NO | | | NO | |
| 2053 | 2018 Jun. 23T18:02:45 | NO | | | NO | |
| 2053 | 2018 Jun. 24T18:03:29 | YES | SEVERE | | YES | 1 |
| 2053 | 2018 Jun. 25T18:03:01 | NO | | | NO | |
| 2053 | 2018 Jun. 26T18:20:39 | NO | | | NO | |
| 2053 | 2018 Jun. 27T18:06:31 | NO | | | NO | |
| 2053 | 2018 Jun. 28T18:00:48 | YES | SEVERE | | YES | 1 |
| 2053 | 2018 Jun. 29T19:23:16 | NO | | | NO | |
| 2053 | 2018 Jun. 30T19:08:56 | YES | SEVERE | | YES | 1 |
| 2053 | 2018 Jul. 1T18:28:35 | NO | | | NO | |
| 2053 | 2018 Jul. 2T18:05:34 | YES | SEVERE | | YES | 1 |
| 2053 | 2018 Jul. 3T18:05:38 | NO | | | NO | |
| 2053 | 2018 Jul. 4T18:00:44 | NO | | | NO | |
| 2053 | 2018 Jul. 5T18:35:31 | NO | | | NO | |
| 2053 | 2018 Jul. 6T18:00:49 | YES | MODERATE | | YES | 1 |
| 2053 | 2018 Jul. 7T18:03:32 | NO | | | NO | |
| 2053 | 2018 Jul. 8T18:15:44 | NO | | | NO | |
| 2053 | 2018 Jul. 9T18:07:25 | NO | | | NO | |
| 2053 | 2018 Jul. 10T18:03:24 | NO | | | NO | |
| 2053 | 2018 Jul. 11T18:05:18 | NO | | | NO | |
| 2053 | 2018 Jul. 12T18:15:30 | NO | | | NO | |
| 2053 | 2018 Jul. 13T18:03:16 | NO | | | NO | |
| 2053 | 2018 Jul. 14T18:20:14 | NO | | | NO | |
| 2053 | 2018 Jul. 15T21:04:48 | NO | | | NO | |
| 2053 | 2018 Jul. 16T18:02:41 | NO | | | NO | |
| 2053 | 2018 Jul. 17T18:31:57 | NO | | | NO | |
| 2053 | 2018 Jul. 18T18:30:41 | NO | | | NO | |
| 2053 | 2018 Jul. 19T18:00:27 | NO | | | NO | |
| 2053 | 2018 Jul. 20T18:05:53 | NO | | | NO | |
| 2053 | 2018 Jul. 21T18:30:46 | YES | SEVERE | | YES | 1 |
| 2053 | 2018 Jul. 22T18:03:43 | NO | | | NO | |
| 2053 | 2018 Jul. 23T18:00:44 | NO | | | NO | |
| 2053 | 2018 Jul. 24T18:02:35 | NO | | | NO | |
| 2053 | 2018 Jul. 25T18:04:44 | NO | | | NO | |
| 2053 | 2018 Jul. 26T18:45:07 | YES | SEVERE | | YES | 1 |
| 2053 | 2018 Jul. 27T18:15:41 | NO | | | NO | |
| 2053 | 2018 Jul. 28T18:54:53 | NO | | | NO | |
| 2053 | 2018 Jul. 29T18:20:43 | NO | | | NO | |
| 2053 | 2018 Jul. 30T18:01:53 | YES | SEVERE | | YES | 1 |
| 2053 | 2018 Jul. 31T18:04:14 | NO | | | NO | |
| 2053 | 2018 Aug. 1T18:08:22 | YES | SEVERE | | YES | 1 |
| 2053 | 2018 Aug. 2T18:11:50 | NO | | | NO | |
| 2053 | 2018 Aug. 3T18:09:40 | NO | | | NO | |
| 2053 | 2018 Aug. 4T18:00:56 | NO | | | NO | |
| 2053 | 2018 Aug. 5T18:00:32 | NO | | | NO | |
| 2053 | 2018 Aug. 6T18:00:39 | NO | | | NO | |
| 2053 | 2018 Aug. 7T18:02:02 | YES | SEVERE | | YES | 1 |
| 2053 | 2018 Aug. 8T18:04:12 | NO | | | NO | |
| 2053 | 2018 Aug. 9T18:55:47 | NO | | | NO | |
| 2053 | 2018 Aug. 10T18:15:40 | NO | | | NO | |
| 2053 | 2018 Aug. 11T18:56:42 | NO | | | NO | |
| 2053 | 2018 Aug. 12T18:31:47 | YES | SEVERE | | YES | 1 |
| 2053 | 2018 Aug. 13T18:43:18 | NO | | | NO | |
| 2053 | 2018 Aug. 14T18:05:37 | NO | | | NO | |
| 2053 | 2018 Aug. 15T18:07:14 | NO | | | NO | |
| 2053 | 2018 Aug. 16T18:01:55 | NO | | | NO | |
| 2053 | 2018 Aug. 17T18:00:16 | NO | | | NO | |
| 2053 | 2018 Aug. 18T18:00:46 | NO | | | NO | |
| 2053 | 2018 Aug. 19T18:00:41 | NO | | | NO | |
| 2053 | 2018 Aug. 20T18:46:19 | YES | SEVERE | | YES | 1 |
| 2053 | 2018 Aug. 21T21:04:08 | NO | | | NO | |
| 2053 | 2018 Aug. 22T18:00:52 | NO | | | NO | |
| 2053 | 2018 Aug. 23T18:00:43 | YES | SEVERE | | YES | 1 |
| 2053 | 2018 Aug. 24T18:02:39 | NO | | | NO | |
| 2053 | 2018 Aug. 25T23:57:50 | NO | | | NO | |
| 2053 | 2018 Aug. 26T18:15:13 | YES | SEVERE | | YES | 1 |
| 2053 | 2018 Aug. 27T18:01:27 | NO | | | NO | |
| 2053 | 2018 Aug. 28T18:01:54 | YES | SEVERE | | YES | 1 |
| 2053 | 2018 Aug. 29T18:19:41 | NO | | | NO | |
| 2053 | 2018 Aug. 30T18:06:49 | NO | | | NO | |
| 2053 | 2018 Aug. 31T18:00:34 | NO | | | NO | |
| 2053 | 2018 Sep. 1T18:01:50 | NO | | | NO | |
| 2053 | 2018 Sep. 2T18:00:50 | YES | SEVERE | | YES | 1 |
| 2053 | 2018 Sep. 3T22:47:48 | NO | | | NO | |
| 2053 | 2018 Sep. 4T23:23:12 | NO | | | NO | |
| 2053 | 2018 Sep. 5T18:00:29 | NO | | | NO | |
| 2053 | 2018 Sep. 6T22:29:55 | NO | | | NO | |
| 2053 | 2018 Sep. 7T18:18:14 | NO | | | NO | |
| 2053 | 2018 Sep. 8T18:01:11 | NO | | | NO | |
| 2053 | 2018 Sep. 9T18:02:46 | NO | | | NO | |
| 2053 | 2018 Sep. 10T18:48:08 | NO | | | NO | |
| 2053 | 2018 Sep. 11T19:13:37 | YES | SEVERE | | YES | 1 |
| 2053 | 2018 Sep. 12T18:26:00 | NO | | | NO | |
| 2053 | 2018 Sep. 13T18:01:34 | NO | | | NO | |
| 2053 | 2018 Sep. 14T18:16:29 | NO | | | NO | |
| 2053 | 2018 Sep. 15T18:02:25 | YES | SEVERE | | YES | 1 |
| 2053 | 2018 Sep. 16T18:01:40 | NO | | | NO | |
| 2053 | 2018 Sep. 17T18:00:44 | NO | | | NO | |
| 2053 | 2018 Sep. 18T18:01:38 | NO | | | NO | |
| 2053 | 2018 Sep. 19T18:01:07 | NO | | | NO | |
| 2053 | 2018 Sep. 20T18:04:18 | NO | | | NO | |
| 2053 | 2018 Sep. 21T18:00:40 | YES | SEVERE | | YES | 1 |
| 2053 | 2018 Sep. 22T18:01:39 | NO | | | NO | |
| 2053 | 2018 Sep. 23T18:01:03 | YES | SEVERE | | YES | 1 |
| 2053 | 2018 Sep. 24T18:00:33 | NO | | | NO | |
| 2053 | 2018 Sep. 25T18:05:09 | NO | | | NO | |
| 2053 | 2018 Sep. 26T18:00:09 | NO | | | NO | |
| 2053 | 2018 Sep. 27T18:00:41 | NO | | | NO | |
| 2053 | 2018 Sep. 28T18:01:36 | YES | SEVERE | | YES | 1 |
| 2053 | 2018 Sep. 29T18:00:36 | NO | | | NO | |
| 2053 | 2018 Sep. 30T18:02:05 | NO | | | NO | |
| 2053 | 2018 Oct. 1T18:01:05 | YES | SEVERE | | YES | 1 |
| 2053 | 2018 Oct. 2T18:01:12 | NO | | | NO | |
| 2053 | 2018 Oct. 3T18:05:49 | NO | | | NO | |
| 2053 | 2018 Oct. 4T18:02:30 | YES | SEVERE | | YES | 1 |
| 2053 | 2018 Oct. 5T18:00:39 | NO | | | NO | |
| 2053 | 2018 Oct. 6T18:01:05 | NO | | | NO | |
| 2053 | 2018 Oct. 7T18:01:26 | YES | SEVERE | | YES | 1 |
| 2053 | 2018 Oct. 8T18:00:38 | YES | SEVERE | | YES | 1 |
| 2053 | 2018 Oct. 9T18:03:52 | NO | | | NO | |
| 2053 | 2018 Oct. 10T18:01:16 | NO | | | NO | |
| 2053 | 2018 Oct. 11T18:00:42 | NO | | | NO | |
| 2053 | 2018 Oct. 12T18:00:33 | NO | | | NO | |
| 2053 | 2018 Oct. 13T18:00:33 | YES | SEVERE | | YES | 1 |
| 2053 | 2018 Oct. 14T18:03:51 | NO | | | NO | |
| 2053 | 2018 Oct. 15T18:02:05 | NO | | | NO | |
| 2053 | 2018 Oct. 16T18:00:33 | NO | | | NO | |
| 2053 | 2018 Oct. 17T18:04:47 | NO | | | NO | |
| 2053 | 2018 Oct. 18T18:21:23 | YES | SEVERE | | YES | 1 |
| 2053 | 2018 Oct. 19T18:03:52 | NO | | | NO | |
| 2053 | 2018 Oct. 20T18:04:32 | NO | | | NO | |
| 2053 | 2018 Oct. 21T18:01:22 | YES | SEVERE | | YES | 1 |
| 2053 | 2018 Oct. 22T18:02:24 | NO | | | NO | |
| 2053 | 2018 Oct. 23T18:01:59 | YES | SEVERE | | YES | 1 |
| 2053 | 2018 Oct. 24T18:01:36 | NO | | | NO | |
| 2053 | 2018 Oct. 25T18:04:38 | NO | | | NO | |
| 2053 | 2018 Oct. 26T18:04:31 | NO | | | NO | |
| 2053 | 2018 Oct. 27T18:04:16 | NO | | | NO | |
| 2053 | 2018 Oct. 28T18:03:40 | YES | SEVERE | | YES | 1 |
| 2053 | 2018 Oct. 29T18:02:22 | NO | | | NO | |
| 2053 | 2018 Oct. 30T18:00:33 | NO | | | NO | |
| 2053 | 2018 Oct. 31T18:00:44 | YES | MODERATE | | YES | 1 |
| 2053 | 2018 Nov. 1T18:00:49 | NO | | | NO | |
| 2053 | 2018 Nov. 2T18:00:29 | NO | | | NO | |
| 2053 | 2018 Nov. 3T18:03:30 | NO | | | NO | |
| 2053 | 2018 Nov. 4T18:02:37 | NO | | | NO | |
| 2053 | 2018 Nov. 5T18:03:44 | NO | | | NO | |
| 2053 | 2018 Nov. 6T18:02:34 | YES | MODERATE | | YES | 1 |
| 2053 | 2018 Nov. 7T18:00:44 | NO | | | NO | |
| 2053 | 2018 Nov. 8T18:00:34 | NO | | | NO | |
| 2053 | 2018 Nov. 9T18:01:58 | NO | | | NO | |
| 2053 | 2018 Nov. 10T18:00:25 | NO | | | NO | |
| 2053 | 2018 Nov. 11T18:01:25 | NO | | | NO | |
| 2053 | 2018 Nov. 12T18:00:35 | NO | | | NO | |
| 2053 | 2018 Nov. 13T18:29:51 | NO | | | NO | |
| 2053 | 2018 Nov. 14T18:04:33 | YES | MODERATE | | YES | 1 |
| 2053 | 2018 Nov. 15T18:00:32 | NO | | | NO | |
| 2053 | 2018 Nov. 16T18:01:15 | NO | | | NO | |
| 2053 | 2018 Nov. 17T18:01:09 | NO | | | NO | |
| 2053 | 2018 Nov. 18T18:05:53 | NO | | | NO | |
| 2053 | 2018 Nov. 19T18:00:38 | NO | | | NO | |
| 2053 | 2018 Nov. 20T18:01:45 | NO | | | NO | |
| 2053 | 2018 Nov. 21T18:15:36 | YES | SEVERE | | YES | 1 |
| 2053 | 2018 Nov. 22T21:27:05 | NO | | | NO | |
| 2053 | 2018 Nov. 23T18:03:14 | NO | | | NO | |
| 2053 | 2018 Nov. 24T22:37:56 | NO | | | NO | |
| 2053 | 2018 Nov. 25T18:04:18 | YES | MODERATE | | YES | 1 |
| 2053 | 2018 Nov. 26T18:02:42 | NO | | | NO | |
| 2053 | 2018 Nov. 27T18:03:40 | YES | MODERATE | | YES | 1 |
| 2053 | 2018 Nov. 28T18:06:15 | NO | | | NO | |
| 2053 | 2018 Nov. 29T18:00:39 | NO | | | NO | |
| 2053 | 2018 Nov. 30T18:00:58 | NO | | | NO | |
| 2053 | 2018 Dec. 1T18:01:07 | NO | | | NO | |
| 2053 | 2018 Dec. 2T18:01:30 | NO | | | NO | |
| 2053 | 2018 Dec. 3T18:00:27 | YES | MODERATE | | YES | 1 |
| 2053 | 2018 Dec. 4T18:00:29 | NO | | | NO | |
| 2053 | 2018 Dec. 5T18:00:20 | NO | | | NO | |
| 2053 | 2018 Dec. 6T18:19:08 | NO | | | NO | |
| 2053 | 2018 Dec. 7T18:13:00 | NO | | | NO | |
| 2053 | 2018 Dec. 8T18:05:12 | YES | SEVERE | | YES | 1 |
| 2053 | 2018 Dec. 9T18:00:21 | NO | | | NO | |
| 2053 | 2018 Dec. 10T18:00:56 | YES | SEVERE | | YES | 1 |
| 2053 | 2018 Dec. 11T18:02:35 | NO | | | NO | |
| 2053 | 2018 Dec. 12T18:10:56 | NO | | | NO | |
| 2053 | 2018 Dec. 13T18:46:35 | NO | | | NO | |
| 2053 | 2018 Dec. 15T22:56:57 | NO | | | NO | |
| 2053 | 2018 Dec. 16T18:49:55 | NO | | | NO | |
| 2053 | 2018 Dec. 17T18:00:49 | NO | | | NO | |
| 2053 | 2018 Dec. 18T23:23:23 | NO | | | NO | |
| 2053 | 2018 Dec. 19T18:02:41 | YES | SEVERE | | YES | 1 |
| 2053 | 2018 Dec. 20T18:00:41 | NO | | | NO | |
| 2053 | 2018 Dec. 21T20:49:00 | YES | SEVERE | | YES | 1 |
| 2053 | 2018 Dec. 22T18:02:16 | NO | | | NO | |
| 2053 | 2018 Dec. 23T19:06:34 | NO | | | NO | |
| 2053 | 2018 Dec. 24T18:17:43 | YES | SEVERE | | YES | 1 |
| 2053 | 2018 Dec. 25T18:04:58 | NO | | | NO | |
| 2053 | 2018 Dec. 26T18:02:26 | NO | | | NO | |
| 2053 | 2018 Dec. 27T18:00:44 | NO | | | NO | |
| 2053 | 2018 Dec. 28T18:09:46 | NO | | | NO | |
| 2053 | 2018 Dec. 29T18:00:32 | YES | SEVERE | | YES | 1 |
| 2053 | 2018 Dec. 30T18:00:41 | NO | | | NO | |
| 2053 | 2018 Dec. 31T18:04:05 | YES | SEVERE | | YES | 1 |
| 2053 | 2019 Jan. 1T18:00:45 | NO | | | NO | |
| 2053 | 2019 Jan. 2T18:01:35 | NO | | | NO | |
| 2053 | 2019 Jan. 3T18:03:04 | YES | SEVERE | | YES | 1 |
| 2053 | 2019 Jan. 4T18:00:43 | NO | | | NO | |
| 2053 | 2019 Jan. 5T18:03:43 | NO | | | NO | |
| 2053 | 2019 Jan. 6T18:04:41 | YES | SEVERE | | YES | 1 |
| 2053 | 2019 Jan. 7T18:00:37 | NO | | | NO | |
| 2053 | 2019 Jan. 8T18:00:33 | NO | | | NO | |
| 2053 | 2019 Jan. 9T18:01:23 | NO | | | NO | |
| 2053 | 2019 Jan. 10T18:00:28 | NO | | | NO | |
| 2053 | 2019 Jan. 11T18:00:32 | NO | | | NO | |
| 2053 | 2019 Jan. 12T18:01:32 | NO | | | NO | |
| 2053 | 2019 Jan. 13T18:00:34 | NO | | | NO | |
| 2053 | 2019 Jan. 14T18:04:25 | YES | SEVERE | | YES | 1 |
| 2053 | 2019 Jan. 15T18:04:13 | NO | | | NO | |
| 2053 | 2019 Jan. 16T18:00:56 | NO | | | NO | |
| 2053 | 2019 Jan. 17T18:01:48 | YES | SEVERE | | YES | 1 |
| 2053 | 2019 Jan. 18T18:04:37 | NO | | | NO | |
| 2053 | 2019 Jan. 19T20:46:51 | NO | | | NO | |
| 2053 | 2019 Jan. 20T18:00:25 | NO | | | NO | |
| 2053 | 2019 Jan. 21T18:00:29 | YES | SEVERE | | YES | 1 |
| 2053 | 2019 Jan. 22T18:01:00 | YES | SEVERE | | YES | 1 |
| 2053 | 2019 Jan. 23T18:18:33 | NO | | | NO | |
| 2053 | 2019 Jan. 24T18:00:09 | NO | | | NO | |
| 2053 | 2019 Jan. 25T18:03:27 | NO | | | NO | |
| 2053 | 2019 Jan. 26T18:00:29 | NO | | | NO | |
| 2053 | 2019 Jan. 27T18:00:37 | YES | SEVERE | | YES | 1 |
| 2053 | 2019 Jan. 28T18:09:05 | NO | | | NO | |
| 2053 | 2019 Jan. 29T18:01:59 | NO | | | NO | |
| 2053 | 2019 Jan. 30T18:01:40 | NO | | | NO | |
| 2053 | 2019 Jan. 31T18:00:39 | NO | | | NO | |
| 2053 | 2019 Feb. 1T18:04:36 | NO | | | NO | |
| 2053 | 2019 Feb. 2T18:01:04 | NO | | | NO | |
| 2053 | 2019 Feb. 3T18:02:00 | NO | | | NO | |
| 2053 | 2019 Feb. 4T18:00:34 | NO | | | NO | |
| 2053 | 2019 Feb. 5T18:01:43 | NO | | | NO | |
| 2053 | 2019 Feb. 6T18:00:35 | NO | | | NO | |
| 2053 | 2019 Feb. 7T18:00:31 | NO | | | NO | |
| 2053 | 2019 Feb. 8T18:00:44 | NO | | | NO | |
| 2053 | 2019 Feb. 9T18:00:46 | YES | SEVERE | | YES | 1 |
| 2053 | 2019 Feb. 10T18:04:57 | YES | MODERATE | | YES | 1 |
| 2053 | 2019 Feb. 11T18:00:37 | NO | | | NO | |
| 2053 | 2019 Feb. 12T18:01:49 | NO | | | NO | |
| 2053 | 2019 Feb. 13T18:00:36 | NO | | | NO | |
| 2053 | 2019 Feb. 14T18:05:31 | NO | | | NO | |
| 2053 | 2019 Feb. 15T18:03:54 | NO | | | NO | |
| 2053 | 2019 Feb. 16T18:00:28 | NO | | | NO | |
| 2053 | 2019 Feb. 17T18:23:40 | YES | MODERATE | | YES | 1 |
| 2053 | 2019 Feb. 18T18:00:29 | NO | | | NO | |
| 2053 | 2019 Feb. 19T18:16:42 | NO | | | NO | |
| 2053 | 2019 Feb. 20T18:02:13 | NO | | | NO | |
| 2053 | 2019 Feb. 21T18:00:31 | YES | SEVERE | | YES | 1 |
| 2053 | 2019 Feb. 22T18:02:17 | NO | | | NO | |
| 2053 | 2019 Feb. 23T18:11:00 | YES | SEVERE | | YES | 1 |
| 2053 | 2019 Feb. 24T18:00:41 | NO | | | NO | |
| 2053 | 2019 Feb. 25T18:15:31 | NO | | | NO | |
| 2053 | 2019 Feb. 26T19:02:42 | YES | SEVERE | | YES | 1 |
| 2053 | 2019 Feb. 27T18:02:34 | NO | | | NO | |
| 2053 | 2019 Feb. 28T18:12:11 | NO | | | NO | |
| 2053 | 2019 Mar. 1T18:02:25 | NO | | | NO | |
| 2053 | 2019 Mar. 3T18:07:49 | NO | | | NO | |
| 2053 | 2019 Mar. 4T18:01:03 | NO | | | NO | |
| 2053 | 2019 Mar. 5T18:05:36 | YES | MODERATE | | YES | 1 |
| 2053 | 2019 Mar. 6T20:34:21 | NO | | | NO | |
| 2053 | 2019 Mar. 7T18:00:36 | NO | | | NO | |
| 2053 | 2019 Mar. 8T18:27:18 | YES | SEVERE | | YES | 1 |
| 2053 | 2019 Mar. 9T18:00:31 | NO | | | NO | |
| 2053 | 2019 Mar. 10T18:17:30 | NO | | | NO | |
| 2053 | 2019 Mar. 11T18:00:04 | YES | SEVERE | | YES | 1 |
| 2053 | 2019 Mar. 12T18:00:45 | YES | SEVERE | | YES | 1 |
| 2053 | 2019 Mar. 13T18:16:38 | YES | SEVERE | | YES | 1 |
| 2053 | 2019 Mar. 14T18:15:29 | NO | | | NO | |
| 2053 | 2019 Mar. 15T18:15:45 | YES | SEVERE | | YES | 1 |
| 2053 | 2019 Mar. 16T18:00:56 | NO | | | NO | |
| 2053 | 2019 Mar. 17T18:00:54 | NO | | | NO | |

Subject 2053 is a 50 year-old Caucasian female with history of 9-14 migraine attacks (without aura) per month since the age of 28. Past medication used to treat her headache has been rizatriptan. Additionally, she has been receiving Botox® injections every three months from 5 Apr. 2018.

TABLE 15

| | | Did you | | Take other | Take study | |
|---|---|---|---|---|---|---|
| | | have a | | med | medication | Number |
| | | migraine | | [Observation | [Treatment | of |
| Subject | Date/Time of Finding | headache? | Severity | phase] | phase] | tablets |
| 2356 | 2018 May 14T18:02:56 | NO | | | | |
| 2356 | 2018 May 15T19:47:37 | NO | | | | |
| 2356 | 2018 May 16T18:02:44 | NO | | | | |
| 2356 | 2018 May 17T18:25:02 | YES | MILD | YES | | |
| 2356 | 2018 May 18T18:02:10 | YES | MODERATE | YES | | |
| 2356 | 2018 May 20T18:00:32 | YES | MODERATE | YES | | |
| 2356 | 2018 May 21T18:30:44 | YES | SEVERE | YES | | |
| 2356 | 2018 May 22T23:29:20 | YES | SEVERE | YES | | |
| 2356 | 2018 May 23T18:05:44 | YES | MODERATE | YES | | |
| 2356 | 2018 May 24T18:02:39 | NO | | | | |
| 2356 | 2018 May 25T20:04:22 | NO | | | | |
| 2356 | 2018 May 26T20:00:04 | NO | | | | |
| 2356 | 2018 May 27T18:15:37 | NO | | | | |
| 2356 | 2018 May 28T18:01:55 | NO | | | | |
| 2356 | 2018 May 29T18:06:37 | YES | SEVERE | YES | | |
| 2356 | 2018 May 30T20:12:13 | YES | SEVERE | YES | | |
| 2356 | 2018 May 31T18:01:40 | YES | MILD | YES | | |
| 2356 | 2018 Jun. 1T18:03:04 | YES | MILD | YES | | |
| 2356 | 2018 Jun. 2T18:32:02 | YES | MILD | YES | | |
| 2356 | 2018 Jun. 3T20:59:50 | YES | MILD | YES | | |
| 2356 | 2018 Jun. 4T18:08:23 | NO | | | | |
| 2356 | 2018 Jun. 5T18:28:44 | YES | MILD | YES | | |
| 2356 | 2018 Jun. 6T18:02:07 | YES | MODERATE | YES | | |
| 2356 | 2018 Jun. 7T18:10:44 | YES | SEVERE | YES | | |
| 2356 | 2018 Jun. 8T23:51:45 | YES | SEVERE | YES | | |
| 2356 | 2018 Jun. 9T18:34:28 | YES | MILD | YES | | |
| 2356 | 2018 Jun. 10T22:40:17 | NO | | | | |
| 2356 | 2018 Jun. 11T18:00:34 | YES | MILD | NO | | |
| 2356 | 2018 Jun. 12T22:51:22 | NO | | | | |
| 2356 | 2018 Jun. 13T18:02:56 | NO | | | | |
| 2356 | 2018 Jun. 14T21:37:44 | NO | | | | |
| 2356 | 2018 Jun. 15T18:34:09 | NO | | | | |
| 2356 | 2018 Jun. 16T21:08:53 | NO | | | | |
| 2356 | 2018 Jun. 17T22:37:17 | NO | | | | |
| 2356 | 2018 Jun. 18T18:06:56 | NO | | | | |
| 2356 | 2018 Jun. 19T18:00:40 | YES | MODERATE | NO | | |
| 2356 | 2018 Jun. 20T18:04:54 | YES | MILD | NO | | |
| 2356 | 2018 Jun. 21 | YES | MILD | | YES | 1 |
| 2356 | 2018 Jun. 22T21:42:36 | NO | | | | |
| 2356 | 2018 Jun. 23 | YES | MODERATE | | YES | 1 |
| 2356 | 2018 Jun. 24T18:49:49 | NO | | | | |
| 2356 | 2018 Jun. 25T18:05:48 | NO | | | NO | |
| 2356 | 2018 Jun. 26T18:17:42 | YES | MILD | | YES | 1 |
| 2356 | 2018 Jun. 27T18:21:01 | YES | MILD | | YES | 1 |
| 2356 | 2018 Jun. 28T18:21:05 | YES | MILD | | NO | |
| 2356 | 2018 Jun. 29T18:00:31 | NO | | | NO | |
| 2356 | 2018 Jun. 30T18:16:22 | YES | MILD | | YES | 1 |
| 2356 | 2018 Jul. 1T18:01:29 | NO | | | NO | |
| 2356 | 2018 Jul. 2T18:01:56 | YES | MODERATE | | YES | 1 |
| 2356 | 2018 Jul. 3T18:15:04 | YES | MODERATE | | YES | 1 |
| 2356 | 2018 Jul. 4T20:00:44 | YES | MODERATE | | NO | |
| 2356 | 2018 Jul. 5T18:04:39 | NO | | | NO | |
| 2356 | 2018 Jul. 6T18:01:24 | YES | MILD | | YES | 1 |
| 2356 | 2018 Jul. 7T18:58:50 | NO | | | NO | |
| 2356 | 2018 Jul. 8T21:41:34 | NO | | | NO | |
| 2356 | 2018 Jul. 9T18:01:41 | YES | MILD | | YES | 1 |
| 2356 | 2018 Jul. 10T18:00:20 | NO | | | NO | |
| 2356 | 2018 Jul. 11T18:00:31 | NO | | | NO | |
| 2356 | 2018 Jul. 12T18:04:18 | YES | MILD | | YES | 1 |
| 2356 | 2018 Jul. 13T18:00:42 | NO | | | NO | |
| 2356 | 2018 Jul. 14T18:04:41 | YES | MILD | | YES | 1 |
| 2356 | 2018 Jul. 15T18:21:18 | NO | | | NO | |
| 2356 | 2018 Jul. 16T20:03:33 | YES | MILD | | YES | 1 |
| 2356 | 2018 Jul. 17T18:05:08 | YES | MODERATE | | YES | 1 |
| 2356 | 2018 Jul. 18T18:17:15 | NO | | | NO | |
| 2356 | 2018 Jul. 19T18:01:42 | YES | MILD | | YES | 1 |
| 2356 | 2018 Jul. 21T18:04:24 | YES | MILD | | YES | 1 |
| 2356 | 2018 Jul. 22T18:42:50 | NO | | | NO | |
| 2356 | 2018 Jul. 23T18:00:54 | YES | MILD | | YES | 1 |
| 2356 | 2018 Jul. 24T18:02:57 | YES | MODERATE | | YES | 1 |
| 2356 | 2018 Jul. 25T21:44:17 | YES | MODERATE | | YES | 1 |
| 2356 | 2018 Jul. 26T18:01:31 | NO | | | NO | |
| 2356 | 2018 Jul. 27T18:00:31 | YES | MODERATE | | YES | 1 |
| 2356 | 2018 Jul. 28T21:39:01 | YES | MILD | | NO | |
| 2356 | 2018 Jul. 29T18:02:26 | YES | MODERATE | | YES | 1 |
| 2356 | 2018 Jul. 30T20:40:53 | YES | MODERATE | | YES | 1 |
| 2356 | 2018 Jul. 31T18:08:16 | YES | MILD | | YES | 1 |
| 2356 | 2018 Aug. 2T19:10:09 | YES | MODERATE | | YES | 1 |
| 2356 | 2018 Aug. 3T20:44:29 | NO | | | NO | |
| 2356 | 2018 Aug. 4T21:54:16 | NO | | | NO | |
| 2356 | 2018 Aug. 5T18:16:03 | YES | MODERATE | | YES | 1 |
| 2356 | 2018 Aug. 6T19:42:00 | YES | MODERATE | | YES | 1 |
| 2356 | 2018 Aug. 7T18:33:22 | YES | MILD | | YES | 1 |
| 2356 | 2018 Aug. 8T18:03:17 | YES | MILD | | YES | 1 |
| 2356 | 2018 Aug. 9T18:02:10 | NO | | | NO | |
| 2356 | 2018 Aug. 10T19:11:15 | YES | MODERATE | | YES | 1 |
| 2356 | 2018 Aug. 12T18:45:22 | YES | MILD | | YES | 1 |
| 2356 | 2018 Aug. 14T18:15:32 | YES | MODERATE | | YES | 1 |
| 2356 | 2018 Aug. 15T20:19:41 | YES | MODERATE | | YES | 1 |
| 2356 | 2018 Aug. 16T18:47:45 | NO | | | NO | |
| 2356 | 2018 Aug. 17T18:00:59 | YES | MILD | | YES | 1 |
| 2356 | 2018 Aug. 18T20:16:02 | NO | | | NO | |
| 2356 | 2018 Aug. 19T18:18:25 | NO | | | NO | |
| 2356 | 2018 Aug. 20T19:38:01 | YES | MILD | | YES | 1 |
| 2356 | 2018 Aug. 21T18:49:37 | YES | MILD | | YES | 1 |
| 2356 | 2018 Aug. 22T18:18:43 | YES | MILD | | YES | 1 |
| 2356 | 2018 Aug. 23T18:01:37 | YES | MODERATE | | YES | 1 |
| 2356 | 2018 Aug. 24T18:07:40 | YES | MODERATE | | YES | 1 |
| 2356 | 2018 Aug. 25T18:14:06 | YES | SEVERE | | YES | 1 |
| 2356 | 2018 Aug. 26T18:20:26 | NO | | | NO | |
| 2356 | 2018 Aug. 27T20:36:51 | YES | MODERATE | | YES | 1 |
| 2356 | 2018 Aug. 28T18:32:12 | YES | MODERATE | | YES | 1 |
| 2356 | 2018 Aug. 29T18:00:30 | YES | MODERATE | | YES | 1 |
| 2356 | 2018 Aug. 30T18:00:44 | YES | MODERATE | | YES | 1 |
| 2356 | 2018 Aug. 31T18:01:42 | YES | MILD | | YES | 1 |
| 2356 | 2018 Sep. 1T20:54:45 | NO | | | NO | |
| 2356 | 2018 Sep. 2T19:37:50 | YES | MILD | | YES | 1 |
| 2356 | 2018 Sep. 3T18:52:43 | NO | | | NO | |
| 2356 | 2018 Sep. 4T18:02:49 | YES | MODERATE | | YES | 1 |
| 2356 | 2018 Sep. 5T18:18:02 | NO | | | NO | |
| 2356 | 2018 Sep. 6T18:00:58 | YES | SEVERE | | YES | 1 |
| 2356 | 2018 Sep. 7T18:52:28 | YES | MILD | | YES | 1 |
| 2356 | 2018 Sep. 8T21:21:03 | NO | | | NO | |
| 2356 | 2018 Sep. 9T18:01:26 | NO | | | NO | |
| 2356 | 2018 Sep. 10T20:17:00 | YES | MODERATE | | YES | 1 |
| 2356 | 2018 Sep. 11T18:47:46 | YES | MILD | | YES | 1 |
| 2356 | 2018 Sep. 12T18:01:21 | YES | MILD | | YES | 1 |
| 2356 | 2018 Sep. 14T21:17:42 | NO | | | NO | |
| 2356 | 2018 Sep. 15T20:25:13 | NO | | | NO | |
| 2356 | 2018 Sep. 16T18:05:44 | YES | MODERATE | | YES | 1 |
| 2356 | 2018 Sep. 17T18:13:32 | YES | MILD | | YES | 1 |
| 2356 | 2018 Sep. 18T18:46:27 | NO | | | NO | |
| 2356 | 2018 Sep. 19T18:16:33 | YES | MODERATE | | YES | 1 |
| 2356 | 2018 Sep. 20T22:53:14 | YES | MILD | | YES | 1 |
| 2356 | 2018 Sep. 21T18:00:53 | YES | MODERATE | | YES | 1 |
| 2356 | 2018 Sep. 22T18:32:09 | YES | MILD | | YES | 1 |
| 2356 | 2018 Sep. 23T18:03:27 | NO | | | NO | |
| 2356 | 2018 Sep. 24T18:15:44 | YES | MILD | | YES | 1 |
| 2356 | 2018 Sep. 25T18:33:48 | YES | SEVERE | | YES | 1 |
| 2356 | 2018 Sep. 26T18:30:07 | NO | | | NO | |
| 2356 | 2018 Sep. 27T18:00:19 | YES | MODERATE | | YES | 1 |
| 2356 | 2018 Sep. 29T19:15:20 | NO | | | NO | |
| 2356 | 2018 Sep. 30T20:58:56 | NO | | | NO | |
| 2356 | 2018 Oct. 1T18:00:47 | NO | | | NO | |
| 2356 | 2018 Oct. 2T20:24:35 | YES | MODERATE | | YES | 1 |
| 2356 | 2018 Oct. 3T18:31:48 | NO | | | NO | |
| 2356 | 2018 Oct. 4T18:18:50 | YES | MILD | | YES | 1 |
| 2356 | 2018 Oct. 5T18:05:42 | NO | | | NO | |
| 2356 | 2018 Oct. 6T18:01:41 | YES | MODERATE | | YES | 1 |
| 2356 | 2018 Oct. 7T18:08:26 | NO | | | NO | |
| 2356 | 2018 Oct. 8T18:00:47 | YES | MODERATE | | YES | 1 |
| 2356 | 2018 Oct. 9T18:02:39 | NO | | | NO | |
| 2356 | 2018 Oct. 10T18:45:22 | NO | | | NO | |
| 2356 | 2018 Oct. 11T18:16:30 | NO | | | NO | |
| 2356 | 2018 Oct. 12T20:24:46 | YES | SEVERE | | YES | 1 |
| 2356 | 2018 Oct. 13T18:04:53 | NO | | | NO | |
| 2356 | 2018 Oct. 14T18:03:05 | YES | MODERATE | | YES | 1 |
| 2356 | 2018 Oct. 15T18:32:44 | NO | | | NO | |
| 2356 | 2018 Oct. 16T18:02:00 | YES | MODERATE | | YES | 1 |
| 2356 | 2018 Oct. 17T18:01:26 | NO | | | NO | |
| 2356 | 2018 Oct. 18 | YES | MILD | | YES | 1 |
| 2356 | 2018 Oct. 19T18:16:18 | NO | | | NO | |
| 2356 | 2018 Oct. 20T20:37:30 | NO | | | NO | |
| 2356 | 2018 Oct. 21T18:31:17 | NO | | | NO | |
| 2356 | 2018 Oct. 22T18:05:20 | NO | | | NO | |
| 2356 | 2018 Oct. 23T18:02:47 | NO | | | NO | |
| 2356 | 2018 Oct. 24T18:00:27 | NO | | | NO | |
| 2356 | 2018 Oct. 25T18:02:05 | NO | | | NO | |
| 2356 | 2018 Oct. 26T18:00:34 | NO | | | NO | |
| 2356 | 2018 Oct. 27T18:01:28 | NO | | | NO | |
| 2356 | 2018 Oct. 28T18:02:38 | NO | | | NO | |
| 2356 | 2018 Oct. 29T18:45:23 | NO | | | NO | |
| 2356 | 2018 Oct. 30T18:01:13 | YES | MODERATE | | YES | 1 |
| 2356 | 2018 Oct. 31T18:33:28 | NO | | | NO | |
| 2356 | 2018 Nov. 1T21:30:50 | YES | MODERATE | | YES | 1 |
| 2356 | 2018 Nov. 2T18:00:47 | YES | MODERATE | | YES | 1 |
| 2356 | 2018 Nov. 3T18:02:04 | NO | | | NO | |
| 2356 | 2018 Nov. 4T18:01:38 | YES | MODERATE | | YES | 1 |
| 2356 | 2018 Nov. 5T18:01:34 | YES | MILD | | YES | 1 |
| 2356 | 2018 Nov. 6T18:00:39 | YES | MODERATE | | YES | 1 |
| 2356 | 2018 Nov. 7T18:02:38 | YES | SEVERE | | YES | 1 |
| 2356 | 2018 Nov. 8T18:15:27 | NO | | | NO | |
| 2356 | 2018 Nov. 9T18:00:27 | YES | SEVERE | | YES | 1 |
| 2356 | 2018 Nov. 10T20:22:40 | YES | MODERATE | | YES | 1 |
| 2356 | 2018 Nov. 11T18:01:35 | YES | MILD | | NO | |
| 2356 | 2018 Nov. 12T18:00:22 | YES | MODERATE | | NO | |
| 2356 | 2018 Nov. 13T18:01:52 | YES | MODERATE | | YES | 1 |
| 2356 | 2018 Nov. 14T21:25:56 | YES | SEVERE | | YES | 1 |
| 2356 | 2018 Nov. 15T18:55:52 | YES | SEVERE | | YES | 1 |
| 2356 | 2018 Nov. 16T18:00:56 | NO | | | NO | |
| 2356 | 2018 Nov. 17T21:12:41 | NO | | | NO | |
| 2356 | 2018 Nov. 18T18:30:43 | YES | MILD | | YES | 1 |
| 2356 | 2018 Nov. 19T18:02:07 | NO | | | NO | |
| 2356 | 2018 Nov. 20T18:00:42 | NO | | | NO | |
| 2356 | 2018 Nov. 21T18:07:44 | NO | | | NO | |
| 2356 | 2018 Nov. 22T18:33:28 | NO | | | NO | |
| 2356 | 2018 Nov. 23T18:02:05 | NO | | | NO | |
| 2356 | 2018 Nov. 24T18:30:52 | NO | | | NO | |
| 2356 | 2018 Nov. 25T18:00:27 | YES | MILD | | YES | 1 |
| 2356 | 2018 Nov. 26T18:18:26 | YES | MILD | | NO | |
| 2356 | 2018 Nov. 27T18:00:34 | YES | MODERATE | | YES | 1 |
| 2356 | 2018 Nov. 28T18:03:11 | YES | SEVERE | | YES | 1 |
| 2356 | 2018 Nov. 29T18:00:26 | NO | | | NO | |
| 2356 | 2018 Nov. 30T21:43:44 | NO | | | NO | |
| 2356 | 2018 Dec. 1T18:00:32 | NO | | | NO | |
| 2356 | 2018 Dec. 2T18:00:26 | YES | MODERATE | | YES | 1 |
| 2356 | 2018 Dec. 3T18:02:40 | NO | | | NO | |
| 2356 | 2018 Dec. 4T18:00:14 | NO | | | NO | |
| 2356 | 2018 Dec. 5T19:26:35 | YES | MILD | | YES | 1 |
| 2356 | 2018 Dec. 6T18:01:32 | NO | | | NO | |
| 2356 | 2018 Dec. 7T18:01:59 | NO | | | NO | |
| 2356 | 2018 Dec. 8T23:10:30 | NO | | | NO | |
| 2356 | 2018 Dec. 9T18:03:45 | NO | | | NO | |
| 2356 | 2018 Dec. 10T18:32:16 | YES | MILD | | YES | 1 |
| 2356 | 2018 Dec. 11T19:45:15 | YES | MILD | | YES | 1 |
| 2356 | 2018 Dec. 12T18:16:36 | NO | | | NO | |
| 2356 | 2018 Dec. 13 | YES | MODERATE | | YES | 1 |
| 2356 | 2018 Dec. 14T18:17:41 | NO | | | NO | |
| 2356 | 2018 Dec. 15T22:42:03 | NO | | | NO | |
| 2356 | 2018 Dec. 16T18:00:04 | YES | MILD | | YES | 1 |
| 2356 | 2018 Dec. 17T18:00:30 | YES | MILD | | YES | 1 |
| 2356 | 2018 Dec. 18T18:17:39 | NO | | | NO | |
| 2356 | 2018 Dec. 19T18:00:36 | YES | MILD | | YES | 1 |
| 2356 | 2018 Dec. 20T18:01:46 | NO | | | NO | |
| 2356 | 2018 Dec. 21T18:05:04 | NO | | | NO | |
| 2356 | 2018 Dec. 22T18:01:21 | NO | | | NO | |
| 2356 | 2018 Dec. 23T18:00:54 | NO | | | NO | |
| 2356 | 2018 Dec. 24T20:19:37 | NO | | | NO | |
| 2356 | 2018 Dec. 25T20:52:55 | YES | MODERATE | | YES | 1 |
| 2356 | 2018 Dec. 26T22:10:45 | YES | MILD | | YES | 1 |
| 2356 | 2018 Dec. 27T18:01:02 | YES | MILD | | YES | 1 |
| 2356 | 2018 Dec. 28T21:22:29 | NO | | | NO | |
| 2356 | 2018 Dec. 29T18:53:46 | NO | | | NO | |
| 2356 | 2018 Dec. 30T18:01:29 | NO | | | NO | |
| 2356 | 2018 Dec. 31T21:33:45 | YES | MODERATE | | YES | 1 |
| 2356 | 2019 Jan. 1T18:02:27 | NO | | | NO | |
| 2356 | 2019 Jan. 2T19:51:24 | NO | | | NO | |
| 2356 | 2019 Jan. 3T20:08:59 | YES | MILD | | YES | 1 |
| 2356 | 2019 Jan. 4 | YES | MODERATE | | YES | 1 |
| 2356 | 2019 Jan. 5T18:03:32 | YES | MODERATE | | NO | |
| 2356 | 2019 Jan. 6T18:02:37 | YES | MODERATE | | YES | 1 |
| 2356 | 2019 Jan. 7T18:00:31 | NO | | | NO | |
| 2356 | 2019 Jan. 8T18:03:02 | YES | MODERATE | | YES | 1 |
| 2356 | 2019 Jan. 9T18:00:55 | NO | | | NO | |
| 2356 | 2019 Jan. 10T18:15:23 | NO | | | NO | |
| 2356 | 2019 Jan. 11T18:01:27 | YES | MODERATE | | YES | 1 |
| 2356 | 2019 Jan. 12T18:02:48 | NO | | | NO | |
| 2356 | 2019 Jan. 13T18:00:32 | NO | | | NO | |
| 2356 | 2019 Jan. 14T18:00:31 | NO | | | NO | |
| 2356 | 2019 Jan. 15T18:00:31 | NO | | | NO | |
| 2356 | 2019 Jan. 16T18:00:35 | YES | MODERATE | | YES | 1 |
| 2356 | 2019 Jan. 17T18:16:32 | NO | | | NO | |
| 2356 | 2019 Jan. 18T18:02:04 | YES | MODERATE | | YES | 1 |
| 2356 | 2019 Jan. 19T18:03:52 | YES | MILD | | YES | 1 |
| 2356 | 2019 Jan. 20T20:40:34 | NO | | | NO | |
| 2356 | 2019 Jan. 21T18:01:48 | YES | MODERATE | | YES | 1 |
| 2356 | 2019 Jan. 22T18:31:31 | YES | MODERATE | | YES | 1 |
| 2356 | 2019 Jan. 24T18:03:26 | YES | MODERATE | | YES | 1 |
| 2356 | 2019 Jan. 25T20:53:10 | NO | | | NO | |
| 2356 | 2019 Jan. 26T23:00:01 | YES | MILD | | YES | 1 |
| 2356 | 2019 Jan. 27T18:00:50 | NO | | | NO | |
| 2356 | 2019 Jan. 28T18:02:15 | NO | | | NO | |
| 2356 | 2019 Jan. 29T18:03:37 | NO | | | NO | |
| 2356 | 2019 Jan. 31T18:00:28 | YES | MODERATE | | YES | 1 |
| 2356 | 2019 Feb. 1T18:02:26 | NO | | | NO | |
| 2356 | 2019 Feb. 2T18:00:20 | NO | | | NO | |
| 2356 | 2019 Feb. 3T18:01:17 | NO | | | NO | |
| 2356 | 2019 Feb. 4T18:00:33 | NO | | | NO | |
| 2356 | 2019 Feb. 5T18:01:17 | NO | | | NO | |
| 2356 | 2019 Feb. 6T19:20:27 | NO | | | NO | |
| 2356 | 2019 Feb. 7T18:02:46 | NO | | | NO | |
| 2356 | 2019 Feb. 8T20:59:04 | NO | | | NO | |
| 2356 | 2019 Feb. 10T18:00:46 | NO | | | NO | |
| 2356 | 2019 Feb. 11T18:01:18 | YES | MILD | | YES | 1 |
| 2356 | 2019 Feb. 12 | YES | MILD | | YES | 1 |
| 2356 | 2019 Feb. 13T20:03:42 | NO | | | NO | |
| 2356 | 2019 Feb. 14T19:03:59 | YES | MILD | | YES | 1 |
| 2356 | 2019 Feb. 15T18:00:34 | YES | MODERATE | | NO | |
| 2356 | 2019 Feb. 16T18:03:05 | YES | MODERATE | | YES | 1 |
| 2356 | 2019 Feb. 17T18:00:35 | YES | MILD | | NO | |
| 2356 | 2019 Feb. 18T18:00:49 | YES | MILD | | YES | 1 |
| 2356 | 2019 Feb. 19T18:04:20 | NO | | | NO | |
| 2356 | 2019 Feb. 20T18:01:14 | YES | MODERATE | | YES | 1 |
| 2356 | 2019 Feb. 21T18:02:20 | YES | MODERATE | | YES | 1 |
| 2356 | 2019 Feb. 22T18:00:32 | NO | | | NO | |
| 2356 | 2019 Feb. 23T18:02:34 | YES | MODERATE | | YES | 1 |
| 2356 | 2019 Feb. 24T18:00:37 | YES | MILD | | NO | |
| 2356 | 2019 Feb. 25T18:01:34 | YES | MODERATE | | YES | 1 |
| 2356 | 2019 Feb. 26T18:00:54 | NO | | | NO | |
| 2356 | 2019 Feb. 27T18:00:23 | YES | MODERATE | | YES | 1 |
| 2356 | 2019 Feb. 28T20:37:22 | NO | | | NO | |
| 2356 | 2019 Mar. 1T18:04:17 | NO | | | NO | |
| 2356 | 2019 Mar. 2T20:49:41 | YES | MILD | | YES | 1 |
| 2356 | 2019 Mar. 3T18:00:40 | YES | SEVERE | | YES | 1 |
| 2356 | 2019 Mar. 4T18:00:31 | NO | | | NO | |
| 2356 | 2019 Mar. 5T18:00:25 | YES | MODERATE | | YES | 1 |
| 2356 | 2019 Mar. 6T20:15:42 | NO | | | NO | |
| 2356 | 2019 Mar. 7T18:00:43 | YES | SEVERE | | YES | 1 |
| 2356 | 2019 Mar. 8T19:46:52 | YES | MILD | | NO | |
| 2356 | 2019 Mar. 9T18:01:34 | YES | SEVERE | | YES | 1 |
| 2356 | 2019 Mar. 10T18:46:31 | NO | | | NO | |
| 2356 | 2019 Mar. 11T18:01:10 | YES | SEVERE | | YES | 1 |
| 2356 | 2019 Mar. 12T18:01:23 | YES | MODERATE | | YES | 1 |
| 2356 | 2019 Mar. 13T18:01:55 | YES | MILD | | NO | |
| 2356 | 2019 Mar. 14T21:11:33 | YES | MODERATE | | NO | |
| 2356 | 2019 Mar. 15T18:01:23 | YES | MILD | | YES | 1 |
| 2356 | 2019 Mar. 16T18:00:56 | NO | | | NO | |
| 2356 | 2019 Mar. 17T18:34:42 | YES | SEVERE | | YES | 1 |
| 2356 | 2019 Mar. 18T18:00:27 | YES | MILD | | YES | 1 |
| 2356 | 2019 Mar. 19T18:03:39 | NO | | | NO | |
| 2356 | 2019 Mar. 20T18:13:40 | YES | MODERATE | | YES | 1 |
| 2356 | 2019 Mar. 21T18:46:31 | YES | MODERATE | | YES | 1 |
| 2356 | 2019 Mar. 22T18:45:32 | YES | MILD | | YES | 1 |
| 2356 | 2019 Mar. 23T23:13:14 | NO | | | NO | |
| 2356 | 2019 Mar. 24T18:03:05 | NO | | | NO | |
| 2356 | 2019 Mar. 25 | YES | MODERATE | | YES | 1 |
| 2356 | 2019 Mar. 26T18:00:54 | NO | | | NO | |
| 2356 | 2019 Mar. 27T21:17:13 | NO | | | NO | |
| 2356 | 2019 Mar. 28T18:01:08 | NO | | | NO | |
| 2356 | 2019 Mar. 29T18:00:36 | YES | MILD | | YES | 1 |
| 2356 | 2019 Mar. 30T18:15:24 | NO | | | NO | |
| 2356 | 2019 Mar. 31T18:01:31 | YES | MODERATE | | YES | 1 |
| 2356 | 2019 Apr. 1T18:02:37 | YES | MILD | | NO | |
| 2356 | 2019 Apr. 2T18:00:37 | YES | MILD | | YES | 1 |
| 2356 | 2019 Apr. 3T18:00:43 | YES | MODERATE | | YES | 1 |
| 2356 | 2019 Apr. 4T18:01:07 | YES | MODERATE | | YES | 1 |
| 2356 | 2019 Apr. 5T18:00:40 | NO | | | NO | |
| 2356 | 2019 Apr. 6T18:00:38 | NO | | | NO | |
| 2356 | 2019 Apr. 7T21:28:48 | NO | | | NO | |
| 2356 | 2019 Apr. 8T18:00:47 | YES | MODERATE | | YES | 1 |
| 2356 | 2019 Apr. 9T18:00:28 | YES | SEVERE | | YES | 1 |
| 2356 | 2019 Apr. 10T18:02:33 | YES | MILD | | NO | |
| 2356 | 2019 Apr. 11T18:00:38 | NO | | | NO | |
| 2356 | 2019 Apr. 12T18:01:05 | YES | MILD | | YES | 1 |
| 2356 | 2019 Apr. 13T18:00:44 | YES | MODERATE | | YES | 1 |
| 2356 | 2019 Apr. 14T18:00:55 | YES | MILD | | YES | 1 |
| 2356 | 2019 Apr. 15T18:00:35 | YES | SEVERE | | YES | 1 |
| 2356 | 2019 Apr. 16T18:02:24 | YES | SEVERE | | NO | |
| 2356 | 2019 Apr. 17T18:00:39 | YES | SEVERE | | YES | 1 |
| 2356 | 2019 Apr. 18T18:00:43 | YES | MILD | | YES | 1 |
| 2356 | 2019 Apr. 19T18:01:32 | NO | | | NO | |
| 2356 | 2019 Apr. 20T18:00:25 | YES | MODERATE | | YES | 1 |
| 2356 | 2019 Apr. 21T18:00:42 | NO | | | NO | |
| 2356 | 2019 Apr. 22T18:30:34 | NO | | | NO | |
| 2356 | 2019 Apr. 23T20:51:03 | YES | MODERATE | | NO | |
| 2356 | 2019 Apr. 24T20:56:08 | YES | MODERATE | | YES | 1 |
| 2356 | 2019 Apr. 25T18:00:29 | YES | MODERATE | | YES | 1 |
| 2356 | 2019 Apr. 26T18:00:44 | NO | | | NO | |
| 2356 | 2019 Apr. 27T18:00:10 | YES | MODERATE | | YES | 1 |
| 2356 | 2019 Apr. 28T21:54:20 | NO | | | NO | |
| 2356 | 2019 Apr. 29T21:40:41 | YES | MILD | | YES | 1 |
| 2356 | 2019 Apr. 30T18:17:56 | YES | MILD | | YES | 1 |
| 2356 | 2019 May 1T22:19:41 | YES | MODERATE | | YES | 1 |
| 2356 | 2019 May 2T20:03:36 | YES | MILD | | YES | 1 |
| 2356 | 2019 May 3T18:00:48 | YES | MILD | | YES | 1 |
| 2356 | 2019 May 4T18:00:51 | NO | | | NO | |
| 2356 | 2019 May 5T18:01:22 | YES | MILD | | YES | 1 |
| 2356 | 2019 May 7T18:15:27 | YES | MILD | | YES | 1 |
| 2356 | 2019 May 8T18:13:02 | YES | MODERATE | | YES | 1 |
| 2356 | 2019 May 9T18:01:16 | YES | MILD | | YES | 1 |
| 2356 | 2019 May 10T18:00:49 | YES | MILD | | NO | |
| 2356 | 2019 May 11T18:01:42 | YES | MODERATE | | YES | 1 |
| 2356 | 2019 May 12T18:00:44 | YES | MODERATE | | NO | |
| 2356 | 2019 May 13T18:03:28 | YES | MILD | | YES | 1 |
| 2356 | 2019 May 14T18:33:27 | YES | MILD | | YES | 1 |
| 2356 | 2019 May 15T18:00:32 | YES | MODERATE | | YES | 1 |
| 2356 | 2019 May 16T18:20:38 | YES | MODERATE | | YES | 1 |
| 2356 | 2019 May 17T18:05:33 | YES | MODERATE | | YES | 1 |
| 2356 | 2019 May 18T19:40:40 | YES | MODERATE | | YES | 1 |
| 2356 | 2019 May 19T18:15:25 | YES | SEVERE | | YES | 1 |
| 2356 | 2019 May 20T18:00:27 | YES | MODERATE | | YES | 1 |
| 2356 | 2019 May 21T18:20:30 | YES | MODERATE | | YES | 1 |
| 2356 | 2019 May 22T18:16:44 | YES | SEVERE | | NO | |
| 2356 | 2019 May 23T18:30:52 | YES | MODERATE | | YES | 1 |
| 2356 | 2019 May 24 | YES | MODERATE | | YES | 1 |
| 2356 | 2019 May 25T18:00:46 | NO | | | NO | |
| 2356 | 2019 May 26T19:14:24 | YES | MODERATE | | YES | 1 |
| 2356 | 2019 May 27T18:17:36 | YES | MODERATE | | YES | 1 |
| 2356 | 2019 May 28T18:31:23 | NO | | | NO | |
| 2356 | 2019 May 30T18:00:49 | NO | | | NO | |
| 2356 | 2019 May 31T20:02:15 | YES | MILD | | YES | 1 |
| 2356 | 2019 Jun. 1T18:01:27 | YES | MILD | | YES | 1 |
| 2356 | 2019 Jun. 2T18:02:22 | YES | MILD | | YES | 1 |
| 2356 | 2019 Jun. 3T18:00:32 | YES | MILD | | YES | 1 |
| 2356 | 2019 Jun. 4T18:00:35 | NO | | | NO | |
| 2356 | 2019 Jun. 5T18:18:32 | NO | | | NO | |
| 2356 | 2019 Jun. 6T18:04:29 | YES | MILD | | YES | 1 |
| 2356 | 2019 Jun. 8T20:32:12 | YES | MILD | | YES | 1 |
| 2356 | 2019 Jun. 9T18:00:46 | NO | | | NO | |
| 2356 | 2019 Jun. 10 | YES | MILD | | YES | 1 |
| 2356 | 2019 Jun. 11T21:01:47 | YES | MILD | | YES | 1 |

Subject 2356 is a 43 year-old Caucasian female with history of 9-14 migraine attacks (with typical aura) per month since the age of 10. Past medication used to treat her headache has been frovatriptan, naproxen sodium, promethazine, paracetamol, ketorolac tromethamine, butalbital, and caffeine. Additionally, she has been receiving Botox® injections every three months from 14 May 2018.

TABLE 16

| | | Did you | | Take other | Take study | |
|---|---|---|---|---|---|---|
| | | have a | | med | medication | Number |
| | | migraine | | [Observation | [Treatment | of |
| Subject | Date/Time of Finding | headache? | Severity | phase] | phase] | tablets |
| 2420 | 2018 May 17T22:59:27 | YES | SEVERE | YES | | |
| 2420 | 2018 May 18T19:18:37 | YES | MODERATE | NO | | |
| 2420 | 2018 May 19T21:56:21 | YES | MODERATE | YES | | |
| 2420 | 2018 May 20T20:51:55 | YES | MODERATE | YES | | |
| 2420 | 2018 May 21T21:06:53 | NO | | | | |
| 2420 | 2018 May 22T21:25:50 | YES | MODERATE | YES | | |
| 2420 | 2018 May 23T19:06:38 | NO | | | | |
| 2420 | 2018 May 24T23:53:02 | YES | MODERATE | YES | | |
| 2420 | 2018 May 25T22:47:43 | YES | MILD | NO | | |
| 2420 | 2018 May 26T23:23:42 | YES | MILD | NO | | |
| 2420 | 2018 May 27 | YES | SEVERE | | NO | |
| 2420 | 2018 May 28T22:01:29 | YES | SEVERE | YES | | |
| 2420 | 2018 May 29T22:39:51 | NO | | | | |
| 2420 | 2018 May 30T20:21:28 | NO | | | | |
| 2420 | 2018 May 31T21:57:27 | YES | MODERATE | YES | | |
| 2420 | 2018 Jun. 1T21:44:51 | YES | MILD | NO | | |
| 2420 | 2018 Jun. 2T19:53:59 | YES | MILD | NO | | |
| 2420 | 2018 Jun. 3T22:34:51 | YES | MODERATE | YES | | |
| 2420 | 2018 Jun. 4T23:22:59 | YES | MILD | NO | | |
| 2420 | 2018 Jun. 7T21:45:06 | NO | | | | |
| 2420 | 2018 Jun. 8T19:21:11 | NO | | | | |
| 2420 | 2018 Jun. 9T22:02:17 | YES | MILD | YES | | |
| 2420 | 2018 Jun. 10T22:40:16 | YES | MODERATE | NO | | |
| 2420 | 2018 Jun. 11T19:59:27 | NO | | | | |
| 2420 | 2018 Jun. 12T22:10:16 | NO | | | | |
| 2420 | 2018 Jun. 14T18:31:19 | NO | | | | |
| 2420 | 2018 Jun. 15T22:03:55 | NO | | | NO | |
| 2420 | 2018 Jun. 16T20:20:32 | NO | | | NO | |
| 2420 | 2018 Jun. 17T19:46:40 | YES | MILD | | NO | |
| 2420 | 2018 Jun. 18T23:26:57 | NO | | | NO | |
| 2420 | 2018 Jun. 19T21:01:09 | NO | | | NO | |
| 2420 | 2018 Jun. 20T22:17:18 | YES | MILD | | NO | |
| 2420 | 2018 Jun. 21T22:51:28 | YES | MODERATE | | NO | |
| 2420 | 2018 Jun. 22T22:31:04 | YES | MILD | | YES | 1 |
| 2420 | 2018 Jun. 23T23:42:03 | NO | | | NO | |
| 2420 | 2018 Jun. 24T21:38:36 | YES | MODERATE | | YES | 1 |
| 2420 | 2018 Jun. 25T23:05:43 | YES | SEVERE | | YES | 1 |
| 2420 | 2018 Jun. 26T22:05:28 | NO | | | NO | |
| 2420 | 2018 Jun. 27T18:30:27 | NO | | | NO | |
| 2420 | 2018 Jun. 28T23:25:50 | YES | MODERATE | | YES | 1 |
| 2420 | 2018 Jun. 29T22:49:35 | NO | | | NO | |
| 2420 | 2018 Jun. 30T22:35:06 | NO | | | NO | |
| 2420 | 2018 Jul. 1T18:33:58 | NO | | | NO | |
| 2420 | 2018 Jul. 2T21:55:51 | NO | | | NO | |
| 2420 | 2018 Jul. 3T22:17:45 | YES | MODERATE | | YES | 1 |
| 2420 | 2018 Jul. 4T23:02:01 | YES | SEVERE | | YES | 1 |
| 2420 | 2018 Jul. 5T22:24:57 | YES | MODERATE | | YES | 1 |
| 2420 | 2018 Jul. 6T22:00:46 | NO | | | NO | |
| 2420 | 2018 Jul. 7T22:23:11 | NO | | | NO | |
| 2420 | 2018 Jul. 8T22:41:33 | YES | MILD | | YES | 1 |
| 2420 | 2018 Jul. 9T22:08:47 | NO | | | NO | |
| 2420 | 2018 Jul. 10T23:25:33 | YES | MODERATE | | YES | 1 |
| 2420 | 2018 Jul. 11T22:45:54 | NO | | | NO | |
| 2420 | 2018 Jul. 12T22:14:30 | NO | | | NO | |
| 2420 | 2018 Jul. 13T22:40:28 | YES | MILD | | NO | |
| 2420 | 2018 Jul. 14T23:55:48 | NO | | | NO | |
| 2420 | 2018 Jul. 15T22:13:12 | NO | | | NO | |
| 2420 | 2018 Jul. 16T22:19:14 | NO | | | NO | |
| 2420 | 2018 Jul. 17T22:17:37 | NO | | | NO | |
| 2420 | 2018 Jul. 18T21:17:22 | NO | | | NO | |
| 2420 | 2018 Jul. 19T22:15:30 | YES | MODERATE | | YES | 1 |
| 2420 | 2018 Jul. 20T22:34:01 | NO | | | NO | |
| 2420 | 2018 Jul. 22T23:21:59 | YES | MILD | | NO | |
| 2420 | 2018 Jul. 24T22:55:56 | YES | MODERATE | | YES | 1 |
| 2420 | 2018 Jul. 25T23:02:10 | NO | | | NO | |
| 2420 | 2018 Jul. 26T22:18:36 | NO | | | NO | |
| 2420 | 2018 Jul. 27T22:42:42 | NO | | | NO | |
| 2420 | 2018 Jul. 28T22:19:27 | NO | | | NO | |
| 2420 | 2018 Jul. 29T22:56:18 | YES | MILD | | NO | |
| 2420 | 2018 Jul. 30T22:48:36 | YES | SEVERE | | YES | 1 |
| 2420 | 2018 Jul. 31T22:28:04 | YES | MODERATE | | YES | 1 |
| 2420 | 2018 Aug. 1T22:18:17 | NO | | | NO | |
| 2420 | 2018 Aug. 2T23:02:15 | NO | | | NO | |
| 2420 | 2018 Aug. 3T22:18:39 | YES | MILD | | YES | 1 |
| 2420 | 2018 Aug. 4T22:18:02 | NO | | | NO | |
| 2420 | 2018 Aug. 5T23:47:57 | YES | MILD | | NO | |
| 2420 | 2018 Aug. 6T21:46:35 | NO | | | NO | |
| 2420 | 2018 Aug. 7T22:14:59 | NO | | | NO | |
| 2420 | 2018 Aug. 8T21:14:32 | NO | | | NO | |
| 2420 | 2018 Aug. 9T22:58:17 | NO | | | NO | |
| 2420 | 2018 Aug. 10T23:05:35 | NO | | | NO | |
| 2420 | 2018 Aug. 11T22:31:36 | YES | MODERATE | | YES | 1 |
| 2420 | 2018 Aug. 12T22:54:55 | YES | MILD | | YES | 1 |
| 2420 | 2018 Aug. 13T22:46:02 | YES | MODERATE | | NO | |
| 2420 | 2018 Aug. 14T23:03:06 | NO | | | NO | |
| 2420 | 2018 Aug. 15T22:55:59 | NO | | | NO | |
| 2420 | 2018 Aug. 16T20:41:38 | YES | MODERATE | | YES | 1 |
| 2420 | 2018 Aug. 17T23:02:13 | NO | | | NO | |
| 2420 | 2018 Aug. 18T23:15:24 | NO | | | NO | |
| 2420 | 2018 Aug. 19T21:52:21 | NO | | | NO | |
| 2420 | 2018 Aug. 20T23:02:18 | NO | | | NO | |
| 2420 | 2018 Aug. 21T23:23:51 | YES | MILD | | YES | 1 |
| 2420 | 2018 Aug. 22T22:52:06 | NO | | | NO | |
| 2420 | 2018 Aug. 23T23:30:19 | NO | | | NO | |
| 2420 | 2018 Aug. 25T23:42:31 | NO | | | NO | |
| 2420 | 2018 Aug. 26T22:14:43 | YES | MILD | | YES | 1 |
| 2420 | 2018 Aug. 27T23:09:02 | NO | | | NO | |
| 2420 | 2018 Aug. 28T23:01:50 | NO | | | NO | |
| 2420 | 2018 Aug. 29T22:23:36 | YES | MODERATE | | YES | 1 |
| 2420 | 2018 Aug. 30T22:32:57 | YES | MILD | | NO | |
| 2420 | 2018 Aug. 31T22:58:21 | NO | | | NO | |
| 2420 | 2018 Sep. 1T23:25:29 | NO | | | NO | |
| 2420 | 2018 Sep. 2T23:21:11 | YES | MODERATE | | YES | 1 |
| 2420 | 2018 Sep. 3T22:59:24 | NO | | | NO | |
| 2420 | 2018 Sep. 4T23:07:41 | NO | | | NO | |
| 2420 | 2018 Sep. 5T21:18:23 | NO | | | NO | |
| 2420 | 2018 Sep. 6T22:46:23 | NO | | | NO | |
| 2420 | 2018 Sep. 7T23:09:35 | YES | MODERATE | | YES | 1 |
| 2420 | 2018 Sep. 8T23:24:29 | YES | SEVERE | | YES | 1 |
| 2420 | 2018 Sep. 9T22:56:10 | NO | | | NO | |
| 2420 | 2018 Sep. 10T23:23:38 | NO | | | NO | |
| 2420 | 2018 Sep. 11T23:50:46 | YES | MODERATE | | YES | 1 |
| 2420 | 2018 Sep. 12T23:22:45 | YES | MILD | | NO | |
| 2420 | 2018 Sep. 13T23:21:38 | NO | | | NO | |
| 2420 | 2018 Sep. 14T23:52:52 | NO | | | NO | |
| 2420 | 2018 Sep. 15T23:16:02 | NO | | | NO | |
| 2420 | 2018 Sep. 17T23:34:31 | NO | | | NO | |
| 2420 | 2018 Sep. 18T23:22:47 | NO | | | NO | |
| 2420 | 2018 Sep. 19T22:01:24 | NO | | | NO | |
| 2420 | 2018 Sep. 20T23:08:30 | NO | | | NO | |
| 2420 | 2018 Sep. 21T22:37:03 | NO | | | NO | |
| 2420 | 2018 Sep. 22T22:35:35 | NO | | | NO | |
| 2420 | 2018 Sep. 23T23:48:39 | NO | | | NO | |
| 2420 | 2018 Sep. 24T22:27:03 | YES | MILD | | YES | 1 |
| 2420 | 2018 Sep. 25T23:53:16 | NO | | | NO | |
| 2420 | 2018 Sep. 26T23:40:48 | NO | | | NO | |
| 2420 | 2018 Sep. 27T22:38:14 | NO | | | NO | |
| 2420 | 2018 Sep. 28T23:58:49 | NO | | | NO | |
| 2420 | 2018 Sep. 29T22:59:13 | NO | | | NO | |
| 2420 | 2018 Sep. 30 | YES | MODERATE | | YES | 1 |
| 2420 | 2018 Oct. 1T22:32:01 | NO | | | NO | |
| 2420 | 2018 Oct. 2T23:22:18 | NO | | | NO | |
| 2420 | 2018 Oct. 3T23:14:47 | NO | | | NO | |
| 2420 | 2018 Oct. 4T23:01:32 | NO | | | NO | |
| 2420 | 2018 Oct. 5T23:33:02 | YES | MILD | | YES | 1 |
| 2420 | 2018 Oct. 6T23:58:42 | NO | | | NO | |
| 2420 | 2018 Oct. 7T23:16:11 | NO | | | NO | |
| 2420 | 2018 Oct. 8T23:21:59 | NO | | | NO | |
| 2420 | 2018 Oct. 9T23:27:18 | NO | | | NO | |
| 2420 | 2018 Oct. 10T22:13:24 | NO | | | NO | |
| 2420 | 2018 Oct. 11T23:28:38 | NO | | | NO | |
| 2420 | 2018 Oct. 13T23:45:54 | YES | MILD | | NO | |
| 2420 | 2018 Oct. 14T23:07:29 | NO | | | NO | |
| 2420 | 2018 Oct. 15T22:31:19 | NO | | | NO | |
| 2420 | 2018 Oct. 16T23:41:08 | NO | | | NO | |
| 2420 | 2018 Oct. 17T23:28:16 | NO | | | NO | |
| 2420 | 2018 Oct. 18T23:57:10 | NO | | | NO | |
| 2420 | 2018 Oct. 19T23:58:00 | NO | | | NO | |
| 2420 | 2018 Oct. 21T22:16:56 | NO | | | NO | |
| 2420 | 2018 Oct. 22T23:43:34 | NO | | | NO | |
| 2420 | 2018 Oct. 23T22:22:59 | NO | | | NO | |
| 2420 | 2018 Oct. 24T23:02:25 | NO | | | NO | |
| 2420 | 2018 Oct. 26T22:51:46 | NO | | | NO | |
| 2420 | 2018 Oct. 27T21:40:02 | YES | MODERATE | | YES | 1 |
| 2420 | 2018 Oct. 29T20:47:14 | NO | | | NO | |
| 2420 | 2018 Oct. 30T22:48:29 | NO | | | NO | |
| 2420 | 2018 Oct. 31T22:32:36 | NO | | | NO | |
| 2420 | 2018 Nov. 1T22:38:04 | YES | MODERATE | | YES | 1 |
| 2420 | 2018 Nov. 2T23:45:32 | NO | | | NO | |
| 2420 | 2018 Nov. 4T22:56:37 | NO | | | NO | |
| 2420 | 2018 Nov. 5T23:34:28 | NO | | | NO | |
| 2420 | 2018 Nov. 6T22:54:59 | NO | | | NO | |
| 2420 | 2018 Nov. 7T23:40:46 | NO | | | NO | |
| 2420 | 2018 Nov. 8T22:59:04 | NO | | | NO | |
| 2420 | 2018 Nov. 9T23:32:44 | NO | | | NO | |
| 2420 | 2018 Nov. 10T23:40:06 | NO | | | NO | |
| 2420 | 2018 Nov. 11T22:59:21 | YES | MODERATE | | YES | 1 |
| 2420 | 2018 Nov. 12T21:39:57 | YES | MILD | | YES | 1 |
| 2420 | 2018 Nov. 13T23:11:21 | NO | | | NO | |
| 2420 | 2018 Nov. 14T23:05:42 | YES | MILD | | YES | 1 |
| 2420 | 2018 Nov. 15T23:41:39 | NO | | | NO | |
| 2420 | 2018 Nov. 17T23:25:34 | NO | | | NO | |
| 2420 | 2018 Nov. 18T22:27:59 | NO | | | NO | |
| 2420 | 2018 Nov. 19T22:37:33 | NO | | | NO | |
| 2420 | 2018 Nov. 20T23:44:55 | NO | | | NO | |
| 2420 | 2018 Nov. 21T22:57:00 | NO | | | NO | |
| 2420 | 2018 Nov. 22T22:13:52 | YES | MILD | | YES | 1 |
| 2420 | 2018 Nov. 23T22:33:17 | NO | | | NO | |
| 2420 | 2018 Nov. 24T23:17:55 | NO | | | NO | |
| 2420 | 2018 Nov. 25T23:06:58 | NO | | | NO | |
| 2420 | 2018 Nov. 26T23:49:44 | NO | | | NO | |
| 2420 | 2018 Nov. 27T22:44:41 | YES | MILD | | YES | 1 |
| 2420 | 2018 Nov. 28T23:36:08 | NO | | | NO | |
| 2420 | 2018 Nov. 29T22:40:48 | YES | MILD | | NO | |
| 2420 | 2018 Nov. 30T22:56:58 | NO | | | NO | |
| 2420 | 2018 Dec. 1T23:45:06 | NO | | | NO | |
| 2420 | 2018 Dec. 3T23:18:25 | NO | | | NO | |
| 2420 | 2018 Dec. 4T23:27:15 | NO | | | NO | |
| 2420 | 2018 Dec. 5T23:29:15 | NO | | | NO | |
| 2420 | 2018 Dec. 6T22:32:08 | NO | | | NO | |
| 2420 | 2018 Dec. 7T23:31:40 | NO | | | NO | |
| 2420 | 2018 Dec. 9T23:48:41 | YES | MODERATE | | YES | 1 |
| 2420 | 2018 Dec. 10T23:11:58 | NO | | | NO | |
| 2420 | 2018 Dec. 11T23:46:44 | NO | | | NO | |
| 2420 | 2018 Dec. 12T23:52:08 | NO | | | NO | |
| 2420 | 2018 Dec. 13T23:31:55 | YES | MODERATE | | YES | 1 |
| 2420 | 2018 Dec. 14T23:12:40 | NO | | | NO | |
| 2420 | 2018 Dec. 15T23:40:37 | NO | | | NO | |
| 2420 | 2018 Dec. 16T23:27:11 | NO | | | NO | |
| 2420 | 2018 Dec. 17T22:31:31 | NO | | | NO | |
| 2420 | 2018 Dec. 18T22:44:02 | NO | | | NO | |
| 2420 | 2018 Dec. 19T23:27:59 | NO | | | NO | |
| 2420 | 2018 Dec. 20T23:55:51 | NO | | | NO | |
| 2420 | 2018 Dec. 21T22:35:10 | NO | | | NO | |
| 2420 | 2018 Dec. 22T23:38:41 | NO | | | NO | |
| 2420 | 2018 Dec. 23T22:50:00 | YES | MODERATE | | YES | 1 |
| 2420 | 2018 Dec. 24T23:07:14 | YES | MODERATE | | YES | 1 |
| 2420 | 2018 Dec. 25T23:37:14 | YES | MILD | | YES | 1 |
| 2420 | 2018 Dec. 26T23:55:25 | YES | MILD | | NO | |
| 2420 | 2018 Dec. 27T23:42:25 | NO | | | NO | |
| 2420 | 2018 Dec. 28T23:47:51 | YES | MILD | | YES | 1 |
| 2420 | 2018 Dec. 29T23:56:54 | YES | MILD | | NO | |
| 2420 | 2018 Dec. 30T22:35:27 | YES | MILD | | YES | 1 |
| 2420 | 2019 Jan. 1T23:22:15 | NO | | | NO | |
| 2420 | 2019 Jan. 2T21:57:50 | YES | MILD | | YES | 1 |
| 2420 | 2019 Jan. 3T21:30:58 | NO | | | NO | |
| 2420 | 2019 Jan. 4T23:27:08 | NO | | | NO | |
| 2420 | 2019 Jan. 5T23:47:26 | NO | | | NO | |
| 2420 | 2019 Jan. 6T22:43:20 | NO | | | NO | |
| 2420 | 2019 Jan. 7T23:08:20 | NO | | | NO | |
| 2420 | 2019 Jan. 8T22:47:38 | NO | | | NO | |
| 2420 | 2019 Jan. 9T22:48:05 | NO | | | NO | |
| 2420 | 2019 Jan. 10T23:32:21 | NO | | | NO | |
| 2420 | 2019 Jan. 11T23:48:18 | NO | | | NO | |
| 2420 | 2019 Jan. 12T23:03:09 | YES | MILD | | YES | 1 |
| 2420 | 2019 Jan. 13T23:55:34 | NO | | | NO | |
| 2420 | 2019 Jan. 16T22:39:10 | NO | | | NO | |
| 2420 | 2019 Jan. 17T23:05:19 | NO | | | NO | |
| 2420 | 2019 Jan. 18T23:09:13 | NO | | | NO | |
| 2420 | 2019 Jan. 19T23:32:31 | NO | | | NO | |
| 2420 | 2019 Jan. 20T21:35:23 | YES | MODERATE | | YES | 1 |
| 2420 | 2019 Jan. 21T22:58:13 | NO | | | NO | |
| 2420 | 2019 Jan. 22T23:38:19 | NO | | | NO | |
| 2420 | 2019 Jan. 23T23:18:43 | NO | | | NO | |
| 2420 | 2019 Jan. 24T23:19:39 | NO | | | NO | |
| 2420 | 2019 Jan. 25T23:49:19 | YES | MILD | | YES | 1 |
| 2420 | 2019 Jan. 26T23:28:41 | NO | | | NO | |
| 2420 | 2019 Jan. 27T23:11:31 | NO | | | NO | |
| 2420 | 2019 Jan. 28T23:03:16 | YES | MILD | | YES | 1 |
| 2420 | 2019 Jan. 29T23:37:49 | NO | | | NO | |
| 2420 | 2019 Jan. 30T23:45:29 | NO | | | NO | |
| 2420 | 2019 Jan. 31T23:09:43 | NO | | | NO | |
| 2420 | 2019 Feb. 1T22:36:14 | YES | MILD | | YES | 1 |
| 2420 | 2019 Feb. 2T23:57:10 | YES | MILD | | NO | |
| 2420 | 2019 Feb. 3T23:46:26 | NO | | | NO | |
| 2420 | 2019 Feb. 4T23:09:36 | NO | | | NO | |
| 2420 | 2019 Feb. 5T21:50:19 | YES | MODERATE | | YES | 1 |
| 2420 | 2019 Feb. 6T23:04:19 | YES | MILD | | NO | |
| 2420 | 2019 Feb. 7T23:16:02 | NO | | | NO | |
| 2420 | 2019 Feb. 8 | YES | MODERATE | | YES | 1 |
| 2420 | 2019 Feb. 9T23:31:33 | NO | | | NO | |
| 2420 | 2019 Feb. 10T23:04:45 | NO | | | NO | |
| 2420 | 2019 Feb. 11T22:32:55 | NO | | | NO | |
| 2420 | 2019 Feb. 12T22:31:39 | NO | | | NO | |
| 2420 | 2019 Feb. 13T23:34:32 | NO | | | NO | |
| 2420 | 2019 Feb. 14T22:44:25 | NO | | | NO | |
| 2420 | 2019 Feb. 15T23:24:17 | YES | MODERATE | | YES | 1 |
| 2420 | 2019 Feb. 16T23:16:05 | YES | MODERATE | | YES | 1 |
| 2420 | 2019 Feb. 17T23:47:53 | NO | | | NO | |
| 2420 | 2019 Feb. 18T22:12:47 | NO | | | NO | |
| 2420 | 2019 Feb. 19T23:20:06 | YES | MILD | | YES | 1 |
| 2420 | 2019 Feb. 22T19:38:09 | NO | | | NO | |
| 2420 | 2019 Feb. 23T21:34:14 | NO | | | NO | |
| 2420 | 2019 Feb. 24T21:23:57 | NO | | | NO | |
| 2420 | 2019 Feb. 25T22:51:38 | NO | | | NO | |
| 2420 | 2019 Feb. 26T21:34:06 | NO | | | NO | |
| 2420 | 2019 Feb. 27T22:52:35 | NO | | | NO | |
| 2420 | 2019 Feb. 28T21:05:51 | NO | | | NO | |
| 2420 | 2019 Mar. 1T22:10:09 | NO | | | NO | |
| 2420 | 2019 Mar. 2T22:23:19 | NO | | | NO | |
| 2420 | 2019 Mar. 3T23:49:46 | NO | | | NO | |
| 2420 | 2019 Mar. 4T23:41:08 | NO | | | NO | |
| 2420 | 2019 Mar. 5T22:52:08 | NO | | | NO | |
| 2420 | 2019 Mar. 7T23:21:27 | NO | | | NO | |
| 2420 | 2019 Mar. 8T22:53:40 | NO | | | NO | |
| 2420 | 2019 Mar. 9T22:32:28 | NO | | | NO | |
| 2420 | 2019 Mar. 10T22:34:05 | NO | | | NO | |
| 2420 | 2019 Mar. 11T23:06:55 | NO | | | NO | |
| 2420 | 2019 Mar. 12T23:17:38 | NO | | | NO | |
| 2420 | 2019 Mar. 13T23:33:58 | NO | | | NO | |
| 2420 | 2019 Mar. 15T23:24:26 | NO | | | NO | |
| 2420 | 2019 Mar. 16T23:30:16 | NO | | | NO | |
| 2420 | 2019 Mar. 17T23:29:48 | NO | | | NO | |
| 2420 | 2019 Mar. 18T22:14:56 | NO | | | NO | |
| 2420 | 2019 Mar. 19T23:00:59 | NO | | | NO | |
| 2420 | 2019 Mar. 20T23:40:04 | NO | | | NO | |
| 2420 | 2019 Mar. 21T23:44:03 | YES | MILD | | YES | 1 |
| 2420 | 2019 Mar. 22T22:35:09 | NO | | | NO | |
| 2420 | 2019 Mar. 23T23:47:05 | NO | | | NO | |
| 2420 | 2019 Mar. 24T23:47:39 | YES | MODERATE | | YES | 1 |
| 2420 | 2019 Mar. 25T23:08:52 | NO | | | NO | |
| 2420 | 2019 Mar. 26T21:40:03 | NO | | | NO | |
| 2420 | 2019 Mar. 28T23:19:45 | NO | | | NO | |
| 2420 | 2019 Mar. 29T22:37:06 | YES | MODERATE | | YES | 1 |
| 2420 | 2019 Mar. 30T23:00:09 | YES | MILD | | YES | 1 |
| 2420 | 2019 Mar. 31T22:34:59 | YES | MILD | | NO | |
| 2420 | 2019 Apr. 1T22:49:17 | NO | | | NO | |
| 2420 | 2019 Apr. 2T22:26:27 | NO | | | NO | |
| 2420 | 2019 Apr. 3T23:46:35 | NO | | | NO | |
| 2420 | 2019 Apr. 4T23:17:56 | NO | | | NO | |
| 2420 | 2019 Apr. 5T22:32:09 | NO | | | NO | |
| 2420 | 2019 Apr. 6T23:37:54 | YES | MODERATE | | YES | 1 |
| 2420 | 2019 Apr. 7T23:49:05 | NO | | | NO | |
| 2420 | 2019 Apr. 8T22:02:47 | NO | | | NO | |
| 2420 | 2019 Apr. 9T23:20:32 | NO | | | NO | |
| 2420 | 2019 Apr. 10T23:47:13 | NO | | | NO | |
| 2420 | 2019 Apr. 11T23:04:07 | YES | MODERATE | | YES | 1 |
| 2420 | 2019 Apr. 12T23:27:05 | NO | | | NO | |
| 2420 | 2019 Apr. 13T23:20:30 | NO | | | NO | |
| 2420 | 2019 Apr. 14T22:49:55 | NO | | | NO | |
| 2420 | 2019 Apr. 15T22:58:38 | NO | | | NO | |
| 2420 | 2019 Apr. 16T22:36:07 | NO | | | NO | |
| 2420 | 2019 Apr. 17T23:26:53 | NO | | | NO | |
| 2420 | 2019 Apr. 18T22:59:38 | NO | | | NO | |
| 2420 | 2019 Apr. 19T23:31:01 | NO | | | NO | |
| 2420 | 2019 Apr. 20T23:46:07 | NO | | | NO | |
| 2420 | 2019 Apr. 21T22:12:02 | NO | | | NO | |
| 2420 | 2019 Apr. 22T23:02:37 | YES | MILD | | NO | |
| 2420 | 2019 Apr. 23 | YES | MODERATE | | YES | 1 |
| 2420 | 2019 Apr. 23T23:14:18 | NO | | | NO | |
| 2420 | 2019 Apr. 24T23:21:00 | NO | | | NO | |
| 2420 | 2019 Apr. 25T22:34:34 | NO | | | NO | |
| 2420 | 2019 Apr. 26T22:44:04 | NO | | | NO | |
| 2420 | 2019 Apr. 27T23:06:16 | NO | | | NO | |
| 2420 | 2019 Apr. 28T23:42:55 | NO | | | NO | |
| 2420 | 2019 Apr. 29T23:47:39 | NO | | | NO | |
| 2420 | 2019 Apr. 30T22:56:49 | NO | | | NO | |
| 2420 | 2019 May 1T23:44:53 | NO | | | NO | |
| 2420 | 2019 May 2T22:52:18 | NO | | | NO | |
| 2420 | 2019 May 5T22:15:33 | NO | | | NO | |
| 2420 | 2019 May 6T22:40:17 | NO | | | NO | |
| 2420 | 2019 May 7T23:29:39 | NO | | | NO | |
| 2420 | 2019 May 8T22:12:35 | NO | | | NO | |
| 2420 | 2019 May 9T23:21:22 | NO | | | NO | |
| 2420 | 2019 May 10T19:53:39 | YES | MILD | | YES | 1 |
| 2420 | 2019 May 11T23:42:42 | YES | MODERATE | | YES | 1 |
| 2420 | 2019 May 12T23:54:58 | YES | MODERATE | | YES | 1 |
| 2420 | 2019 May 13T23:15:35 | YES | MILD | | YES | 1 |
| 2420 | 2019 May 14T22:51:31 | NO | | | NO | |
| 2420 | 2019 May 15T23:35:59 | NO | | | NO | |
| 2420 | 2019 May 16T22:54:42 | YES | MILD | | NO | |
| 2420 | 2019 May 17T23:46:27 | NO | | | NO | |
| 2420 | 2019 May 18T23:01:26 | NO | | | NO | |
| 2420 | 2019 May 19T23:28:28 | YES | MILD | | YES | 1 |
| 2420 | 2019 May 20T23:29:28 | NO | | | NO | |
| 2420 | 2019 May 21T23:13:51 | NO | | | NO | |
| 2420 | 2019 May 22T23:35:20 | NO | | | NO | |
| 2420 | 2019 May 23T23:21:19 | NO | | | NO | |
| 2420 | 2019 May 24T23:02:42 | NO | | | NO | |
| 2420 | 2019 May 25T22:58:18 | NO | | | NO | |
| 2420 | 2019 May 27T22:58:16 | NO | | | NO | |
| 2420 | 2019 May 28T23:44:28 | NO | | | NO | |
| 2420 | 2019 May 29T23:12:49 | YES | MILD | | YES | 1 |
| 2420 | 2019 May 30T23:37:22 | YES | MODERATE | | YES | 1 |
| 2420 | 2019 May 31T23:54:26 | YES | MILD | | NO | |
| 2420 | 2019 Jun. 1T20:46:32 | YES | MILD | | NO | |
| 2420 | 2019 Jun. 2T21:45:01 | NO | | | NO | |
| 2420 | 2019 Jun. 3T23:49:09 | NO | | | NO | |
| 2420 | 2019 Jun. 4T23:48:27 | NO | | | NO | |
| 2420 | 2019 Jun. 5T23:14:18 | NO | | | NO | |
| 2420 | 2019 Jun. 6T23:51:48 | YES | MILD | | YES | 1 |
| 2420 | 2019 Jun. 9T22:35:28 | NO | | | NO | |
| 2420 | 2019 Jun. 10T23:49:17 | NO | | | NO | |
| 2420 | 2019 Jun. 11T22:44:19 | YES | MILD | | YES | 1 |
| 2420 | 2019 Jun. 12T23:32:56 | NO | | | NO | |
| 2420 | 2019 Jun. 13T23:33:20 | NO | | | NO | |
| 2420 | 2019 Jun. 14T18:14:49 | NO | | | NO | |
| 2420 | 2019 Jun. 15T18:11:31 | NO | | | NO | |
| 2420 | 2019 Jun. 16T21:46:49 | NO | | | NO | |
| 2420 | 2019 Jun. 17T23:11:47 | NO | | | NO | |
| 2420 | 2019 Jun. 18T22:45:26 | NO | | | NO | |
| 2420 | 2019 Jun. 19T23:27:58 | NO | | | NO | |

Subject 2420 is a 35 year-old Caucasian female with history of 9-14 migraine attacks (without aura) per month since the age of 13. Past medication used to treat her headache has been frovatriptan, rizatriptan, paracetamol, fioricet, ibuprofen, and gabapentin. Additionally, she has been receiving Botox® injections every three months from January 2018.

TABLE 17

| | | Did you | | Take other | Take study | |
|---|---|---|---|---|---|---|
| | | have a | | med | medication | Number |
| | | migraine | | [Observation | [Treatment | of |
| Subject | Date/Time of Finding | headache? | Severity | phase] | phase] | tablets |
| 1777 | 2018 Feb. 23T18:00:51 | YES | MODERATE | YES | | |
| 1777 | 2018 Feb. 24T18:01:36 | YES | MODERATE | YES | | |
| 1777 | 2018 Feb. 25T18:02:01 | NO | | | | |
| 1777 | 2018 Feb. 26T18:01:28 | YES | MILD | YES | | |
| 1777 | 2018 Feb. 27T20:17:54 | YES | MILD | YES | | |
| 1777 | 2018 Feb. 28T18:00:32 | YES | MODERATE | YES | | |
| 1777 | 2018 Mar. 1T18:16:38 | NO | | | | |
| 1777 | 2018 Mar. 2T18:01:17 | YES | MILD | YES | | |
| 1777 | 2018 Mar. 3T18:00:35 | NO | | | | |
| 1777 | 2018 Mar. 4T18:00:36 | NO | | | | |
| 1777 | 2018 Mar. 5T18:03:27 | NO | | | | |
| 1777 | 2018 Mar. 6T18:00:26 | NO | | | | |
| 1777 | 2018 Mar. 7T18:00:29 | NO | | | | |
| 1777 | 2018 Mar. 8T18:00:10 | NO | | | | |
| 1777 | 2018 Mar. 9T23:21:26 | YES | MILD | YES | | |
| 1777 | 2018 Mar. 10T18:00:40 | YES | MILD | YES | | |
| 1777 | 2018 Mar. 11T18:00:33 | YES | MILD | YES | | |
| 1777 | 2018 Mar. 12T18:00:39 | YES | MILD | YES | | |
| 1777 | 2018 Mar. 13T18:18:23 | YES | MODERATE | YES | | |
| 1777 | 2018 Mar. 14T18:57:14 | YES | MODERATE | YES | | |
| 1777 | 2018 Mar. 15T18:17:34 | YES | MODERATE | YES | | |
| 1777 | 2018 Mar. 16T20:36:14 | NO | | | | |
| 1777 | 2018 Mar. 17T18:00:25 | NO | | | | |
| 1777 | 2018 Mar. 18T18:00:39 | NO | | | | |
| 1777 | 2018 Mar. 19T18:00:33 | NO | | | | |
| 1777 | 2018 Mar. 20T18:00:49 | YES | MODERATE | YES | | |
| 1777 | 2018 Mar. 21T18:00:32 | YES | MILD | YES | | |
| 1777 | 2018 Mar. 22T18:00:27 | YES | MODERATE | YES | | |
| 1777 | 2018 Mar. 23T18:00:36 | NO | | | NO | |
| 1777 | 2018 Mar. 24T18:31:42 | NO | | | NO | |
| 1777 | 2018 Mar. 25T18:01:36 | NO | | | NO | |
| 1777 | 2018 Mar. 26T18:11:03 | NO | | | NO | |
| 1777 | 2018 Mar. 27T18:00:50 | NO | | | NO | |
| 1777 | 2018 Mar. 28T18:15:10 | NO | | | NO | |
| 1777 | 2018 Mar. 29T18:00:34 | NO | | | NO | |
| 1777 | 2018 Mar. 30T18:00:40 | NO | | | NO | |
| 1777 | 2018 Mar. 31T18:00:27 | YES | MODERATE | | YES | 1 |
| 1777 | 2018 Apr. 1T18:00:45 | NO | | | NO | |
| 1777 | 2018 Apr. 2T18:00:28 | YES | MILD | | YES | 1 |
| 1777 | 2018 Apr. 3T18:10:49 | NO | | | NO | |
| 1777 | 2018 Apr. 4T18:15:24 | NO | | | NO | |
| 1777 | 2018 Apr. 5T19:16:07 | NO | | | NO | |
| 1777 | 2018 Apr. 6T18:00:50 | NO | | | NO | |
| 1777 | 2018 Apr. 7T18:15:51 | NO | | | NO | |
| 1777 | 2018 Apr. 8T18:00:33 | YES | MILD | | YES | 1 |
| 1777 | 2018 Apr. 9T18:01:06 | NO | | | NO | |
| 1777 | 2018 Apr. 11T18:00:29 | NO | | | NO | |
| 1777 | 2018 Apr. 12T22:11:31 | NO | | | NO | |
| 1777 | 2018 Apr. 13T19:34:13 | NO | | | NO | |
| 1777 | 2018 Apr. 14T18:00:39 | YES | MILD | | YES | 1 |
| 1777 | 2018 Apr. 15T18:00:35 | NO | | | NO | |
| 1777 | 2018 Apr. 16T18:00:46 | NO | | | NO | |
| 1777 | 2018 Apr. 17T21:57:34 | NO | | | NO | |
| 1777 | 2018 Apr. 18T18:01:36 | NO | | | NO | |
| 1777 | 2018 Apr. 19T21:53:31 | YES | MILD | | YES | 1 |
| 1777 | 2018 Apr. 20T21:01:29 | NO | | | NO | |
| 1777 | 2018 Apr. 21T21:33:57 | YES | MILD | | YES | 1 |
| 1777 | 2018 Apr. 22T18:00:31 | NO | | | NO | |
| 1777 | 2018 Apr. 23T18:00:43 | YES | MODERATE | | YES | 1 |
| 1777 | 2018 Apr. 24T22:11:15 | NO | | | NO | |
| 1777 | 2018 Apr. 25T18:01:06 | NO | | | NO | |
| 1777 | 2018 Apr. 26T21:42:42 | NO | | | NO | |
| 1777 | 2018 Apr. 27T18:00:02 | YES | MILD | | YES | 1 |
| 1777 | 2018 Apr. 28T18:00:41 | NO | | | NO | |
| 1777 | 2018 Apr. 29T18:24:42 | NO | | | NO | |
| 1777 | 2018 Apr. 30T18:44:51 | NO | | | NO | |
| 1777 | 2018 May 1T21:38:32 | NO | | | NO | |
| 1777 | 2018 May 2T18:00:35 | YES | MODERATE | | YES | 1 |
| 1777 | 2018 May 3T18:02:45 | YES | MODERATE | | YES | 1 |
| 1777 | 2018 May 4T18:00:46 | NO | | | NO | |
| 1777 | 2018 May 5T19:20:10 | NO | | | NO | |
| 1777 | 2018 May 6T18:00:28 | NO | | | NO | |
| 1777 | 2018 May 7T18:00:32 | YES | MODERATE | | YES | 1 |
| 1777 | 2018 May 8T18:00:46 | NO | | | NO | |
| 1777 | 2018 May 9T18:00:03 | NO | | | NO | |
| 1777 | 2018 May 10T18:00:43 | NO | | | NO | |
| 1777 | 2018 May 11T18:00:33 | YES | MODERATE | | YES | 1 |
| 1777 | 2018 May 12 | YES | MILD | | YES | 1 |
| 1777 | 2018 May 12T18:19:51 | NO | | | NO | |
| 1777 | 2018 May 13T18:00:36 | YES | MODERATE | | YES | 1 |
| 1777 | 2018 May 14T18:00:55 | NO | | | NO | |
| 1777 | 2018 May 15T18:00:13 | NO | | | NO | |
| 1777 | 2018 May 16T18:00:31 | NO | | | NO | |
| 1777 | 2018 May 17T20:37:18 | YES | MILD | | YES | 1 |
| 1777 | 2018 May 18T18:00:37 | NO | | | NO | |
| 1777 | 2018 May 19T18:00:37 | YES | MODERATE | | YES | 1 |
| 1777 | 2018 May 20T18:01:06 | NO | | | NO | |
| 1777 | 2018 May 21T18:24:56 | NO | | | NO | |
| 1777 | 2018 May 22T18:01:27 | YES | MILD | | YES | 1 |
| 1777 | 2018 May 23T18:00:01 | NO | | | NO | |
| 1777 | 2018 May 24T21:37:43 | NO | | | NO | |
| 1777 | 2018 May 25T18:00:44 | YES | MILD | | YES | 1 |
| 1777 | 2018 May 26T18:06:38 | YES | MILD | | YES | 1 |
| 1777 | 2018 May 27T18:23:13 | NO | | | NO | |
| 1777 | 2018 May 28T21:33:51 | NO | | | NO | |
| 1777 | 2018 May 29T18:00:50 | YES | MILD | | YES | 1 |
| 1777 | 2018 May 30T18:00:32 | NO | | | NO | |
| 1777 | 2018 May 31T18:00:29 | NO | | | NO | |
| 1777 | 2018 Jun. 1T18:00:28 | NO | | | NO | |
| 1777 | 2018 Jun. 2T23:35:48 | NO | | | NO | |
| 1777 | 2018 Jun. 3T18:03:16 | YES | MODERATE | | YES | 1 |
| 1777 | 2018 Jun. 4T18:00:28 | NO | | | NO | |
| 1777 | 2018 Jun. 5T18:00:22 | NO | | | NO | |
| 1777 | 2018 Jun. 6T18:03:23 | NO | | | NO | |
| 1777 | 2018 Jun. 7T18:00:42 | NO | | | NO | |
| 1777 | 2018 Jun. 8T18:00:30 | NO | | | NO | |
| 1777 | 2018 Jun. 9T18:00:45 | YES | MODERATE | | YES | 1 |
| 1777 | 2018 Jun. 10T18:00:35 | NO | | | NO | |
| 1777 | 2018 Jun. 11T18:00:37 | YES | MILD | | YES | 1 |
| 1777 | 2018 Jun. 12T18:00:38 | NO | | | NO | |
| 1777 | 2018 Jun. 13T18:00:31 | NO | | | NO | |
| 1777 | 2018 Jun. 14T18:00:32 | NO | | | NO | |
| 1777 | 2018 Jun. 15T18:00:26 | YES | MILD | | YES | 1 |
| 1777 | 2018 Jun. 16T19:13:03 | NO | | | NO | |
| 1777 | 2018 Jun. 17T20:57:59 | YES | MODERATE | | YES | 1 |
| 1777 | 2018 Jun. 18T18:00:04 | NO | | | NO | |
| 1777 | 2018 Jun. 19T18:00:40 | NO | | | NO | |
| 1777 | 2018 Jun. 20T18:00:22 | NO | | | NO | |
| 1777 | 2018 Jun. 21T18:02:07 | YES | MODERATE | | YES | 1 |
| 1777 | 2018 Jun. 22T18:03:04 | NO | | | NO | |
| 1777 | 2018 Jun. 23T18:48:51 | NO | | | NO | |
| 1777 | 2018 Jun. 24T18:19:09 | NO | | | NO | |
| 1777 | 2018 Jun. 25T18:01:27 | NO | | | NO | |
| 1777 | 2018 Jun. 26T20:49:32 | YES | MODERATE | | YES | 1 |
| 1777 | 2018 Jun. 27T18:00:35 | NO | | | NO | |
| 1777 | 2018 Jun. 28T18:00:43 | YES | MODERATE | | YES | 1 |
| 1777 | 2018 Jun. 29T18:01:04 | NO | | | NO | |
| 1777 | 2018 Jun. 30T18:04:02 | YES | MILD | | YES | 1 |
| 1777 | 2018 Jul. 1T18:00:33 | NO | | | NO | |
| 1777 | 2018 Jul. 2T18:00:36 | YES | MILD | | YES | 1 |
| 1777 | 2018 Jul. 3T18:00:55 | NO | | | NO | |
| 1777 | 2018 Jul. 4T19:24:13 | YES | MODERATE | | YES | 1 |
| 1777 | 2018 Jul. 5T18:14:50 | NO | | | NO | |
| 1777 | 2018 Jul. 6T18:01:45 | NO | | | NO | |
| 1777 | 2018 Jul. 7T18:30:37 | YES | MILD | | YES | 1 |
| 1777 | 2018 Jul. 8T18:03:41 | NO | | | NO | |
| 1777 | 2018 Jul. 9T18:00:32 | NO | | | NO | |
| 1777 | 2018 Jul. 10T19:04:19 | YES | MILD | | YES | 1 |
| 1777 | 2018 Jul. 11T21:53:17 | NO | | | NO | |
| 1777 | 2018 Jul. 12T18:00:27 | YES | SEVERE | | YES | 1 |
| 1777 | 2018 Jul. 13T18:00:31 | NO | | | NO | |
| 1777 | 2018 Jul. 14T20:31:21 | NO | | | NO | |
| 1777 | 2018 Jul. 15T18:33:39 | NO | | | NO | |
| 1777 | 2018 Jul. 16T18:03:10 | NO | | | NO | |
| 1777 | 2018 Jul. 17T18:00:26 | NO | | | NO | |
| 1777 | 2018 Jul. 18T20:38:43 | NO | | | NO | |
| 1777 | 2018 Jul. 19T18:18:21 | YES | MILD | | YES | 1 |
| 1777 | 2018 Jul. 20T18:53:16 | NO | | | NO | |
| 1777 | 2018 Jul. 21T18:00:42 | YES | MODERATE | | YES | 1 |
| 1777 | 2018 Jul. 22T18:00:42 | NO | | | NO | |
| 1777 | 2018 Jul. 23T18:00:30 | NO | | | NO | |
| 1777 | 2018 Jul. 24T18:00:15 | NO | | | NO | |
| 1777 | 2018 Jul. 25T18:00:41 | NO | | | NO | |
| 1777 | 2018 Jul. 26T18:00:40 | NO | | | NO | |
| 1777 | 2018 Jul. 27T18:00:28 | YES | MILD | | YES | 1 |
| 1777 | 2018 Jul. 28T21:00:34 | NO | | | NO | |
| 1777 | 2018 Jul. 29T18:00:42 | NO | | | NO | |
| 1777 | 2018 Jul. 30T18:00:02 | YES | MILD | | NO | |
| 1777 | 2018 Jul. 31T18:00:40 | YES | MILD | | YES | 1 |
| 1777 | 2018 Aug. 1T18:00:39 | NO | | | NO | |
| 1777 | 2018 Aug. 2T18:00:26 | NO | | | NO | |
| 1777 | 2018 Aug. 3T18:00:24 | NO | | | NO | |
| 1777 | 2018 Aug. 4T20:57:55 | NO | | | NO | |
| 1777 | 2018 Aug. 5T18:00:28 | NO | | | NO | |
| 1777 | 2018 Aug. 6T18:00:34 | NO | | | NO | |
| 1777 | 2018 Aug. 7T19:35:16 | NO | | | NO | |
| 1777 | 2018 Aug. 8T18:00:53 | YES | MODERATE | | YES | 1 |
| 1777 | 2018 Aug. 9T18:00:30 | NO | | | NO | |
| 1777 | 2018 Aug. 10T18:00:38 | YES | MODERATE | | YES | 1 |
| 1777 | 2018 Aug. 11T18:20:21 | NO | | | NO | |
| 1777 | 2018 Aug. 12T20:11:32 | YES | MODERATE | | YES | 1 |
| 1777 | 2018 Aug. 13T18:47:49 | NO | | | NO | |
| 1777 | 2018 Aug. 14T18:00:47 | YES | MODERATE | | YES | 1 |
| 1777 | 2018 Aug. 15T18:00:36 | NO | | | NO | |
| 1777 | 2018 Aug. 16T18:17:55 | NO | | | NO | |
| 1777 | 2018 Aug. 17T18:26:10 | NO | | | NO | |
| 1777 | 2018 Aug. 18T19:25:51 | YES | MODERATE | | YES | 1 |
| 1777 | 2018 Aug. 19T18:00:40 | NO | | | NO | |
| 1777 | 2018 Aug. 20T18:00:48 | YES | MILD | | YES | 1 |
| 1777 | 2018 Aug. 21T18:00:29 | NO | | | NO | |
| 1777 | 2018 Aug. 22T19:13:34 | YES | MODERATE | | YES | 1 |
| 1777 | 2018 Aug. 23T20:22:29 | NO | | | NO | |
| 1777 | 2018 Aug. 24T18:22:10 | NO | | | NO | |
| 1777 | 2018 Aug. 25T18:15:39 | YES | MODERATE | | YES | 1 |
| 1777 | 2018 Aug. 26T18:00:41 | NO | | | NO | |
| 1777 | 2018 Aug. 27T18:27:46 | NO | | | NO | |
| 1777 | 2018 Aug. 28T18:00:37 | YES | MILD | | YES | 1 |
| 1777 | 2018 Aug. 29T18:25:39 | NO | | | NO | |
| 1777 | 2018 Aug. 30T18:17:09 | NO | | | NO | |
| 1777 | 2018 Aug. 31T18:00:37 | YES | MODERATE | | YES | 1 |
| 1777 | 2018 Sep. 1T19:24:49 | NO | | | NO | |
| 1777 | 2018 Sep. 2T20:35:51 | NO | | | NO | |
| 1777 | 2018 Sep. 3T18:26:12 | NO | | | NO | |
| 1777 | 2018 Sep. 4T18:01:32 | YES | MILD | | YES | 1 |
| 1777 | 2018 Sep. 5T18:00:50 | NO | | | NO | |
| 1777 | 2018 Sep. 6T18:55:34 | YES | MILD | | YES | 1 |
| 1777 | 2018 Sep. 7T18:00:27 | NO | | | NO | |
| 1777 | 2018 Sep. 8T18:47:23 | NO | | | NO | |
| 1777 | 2018 Sep. 9T18:17:34 | NO | | | NO | |
| 1777 | 2018 Sep. 10T18:03:22 | NO | | | NO | |
| 1777 | 2018 Sep. 11T18:11:31 | YES | MODERATE | | YES | 1 |
| 1777 | 2018 Sep. 12T18:00:19 | YES | MILD | | YES | 1 |
| 1777 | 2018 Sep. 13T20:48:53 | NO | | | NO | |
| 1777 | 2018 Sep. 14T18:00:22 | NO | | | NO | |
| 1777 | 2018 Sep. 15T22:28:36 | YES | MODERATE | | YES | 1 |
| 1777 | 2018 Sep. 16T18:04:19 | NO | | | NO | |
| 1777 | 2018 Sep. 17T18:00:02 | NO | | | NO | |
| 1777 | 2018 Sep. 18T18:46:54 | YES | SEVERE | | YES | 1 |
| 1777 | 2018 Sep. 19T18:00:29 | NO | | | NO | |
| 1777 | 2018 Sep. 20T18:00:29 | NO | | | NO | |
| 1777 | 2018 Sep. 21T18:22:55 | YES | MODERATE | | YES | 1 |
| 1777 | 2018 Sep. 22T18:55:59 | NO | | | NO | |
| 1777 | 2018 Sep. 23T18:38:40 | NO | | | NO | |
| 1777 | 2018 Sep. 24T18:30:34 | NO | | | NO | |
| 1777 | 2018 Sep. 25T18:20:33 | NO | | | NO | |
| 1777 | 2018 Sep. 26T18:23:08 | YES | MILD | | YES | 1 |
| 1777 | 2018 Sep. 27T18:00:28 | NO | | | NO | |
| 1777 | 2018 Sep. 28T18:03:40 | NO | | | NO | |
| 1777 | 2018 Sep. 29T18:00:03 | NO | | | NO | |
| 1777 | 2018 Sep. 30T18:01:06 | YES | MODERATE | | YES | 1 |
| 1777 | 2018 Oct. 1T18:03:46 | NO | | | NO | |
| 1777 | 2018 Oct. 2T18:57:23 | NO | | | NO | |
| 1777 | 2018 Oct. 3T18:04:55 | NO | | | NO | |
| 1777 | 2018 Oct. 4T18:24:07 | YES | MILD | | YES | 1 |
| 1777 | 2018 Oct. 5T19:04:43 | NO | | | NO | |
| 1777 | 2018 Oct. 6T18:00:22 | NO | | | NO | |
| 1777 | 2018 Oct. 7T18:00:25 | NO | | | NO | |
| 1777 | 2018 Oct. 8T18:00:35 | YES | MILD | | YES | 1 |
| 1777 | 2018 Oct. 9T18:08:47 | NO | | | NO | |
| 1777 | 2018 Oct. 10T18:00:39 | NO | | | NO | |
| 1777 | 2018 Oct. 11T18:00:33 | YES | MODERATE | | YES | 1 |
| 1777 | 2018 Oct. 12T18:00:38 | NO | | | NO | |
| 1777 | 2018 Oct. 13T18:19:29 | NO | | | NO | |
| 1777 | 2018 Oct. 14T18:01:00 | YES | MILD | | YES | 1 |
| 1777 | 2018 Oct. 15T18:00:46 | YES | MILD | | YES | 1 |
| 1777 | 2018 Oct. 16T18:00:39 | NO | | | NO | |
| 1777 | 2018 Oct. 17T18:00:35 | YES | MILD | | YES | 1 |
| 1777 | 2018 Oct. 18T19:07:23 | NO | | | NO | |
| 1777 | 2018 Oct. 19T18:04:22 | YES | MILD | | YES | 1 |
| 1777 | 2018 Oct. 20T18:00:26 | NO | | | NO | |
| 1777 | 2018 Oct. 21T18:00:49 | NO | | | NO | |
| 1777 | 2018 Oct. 22T18:03:29 | YES | MILD | | YES | 1 |
| 1777 | 2018 Oct. 23T18:00:27 | NO | | | NO | |
| 1777 | 2018 Oct. 24T18:00:27 | NO | | | NO | |
| 1777 | 2018 Oct. 25T18:25:00 | YES | MODERATE | | YES | 1 |
| 1777 | 2018 Oct. 26T22:16:58 | YES | MILD | | YES | 1 |
| 1777 | 2018 Oct. 27T18:00:22 | NO | | | NO | |
| 1777 | 2018 Oct. 28T18:07:39 | NO | | | NO | |
| 1777 | 2018 Oct. 29T18:00:03 | NO | | | NO | |
| 1777 | 2018 Oct. 30T18:00:46 | YES | MILD | | YES | 1 |
| 1777 | 2018 Oct. 31T18:00:27 | NO | | | NO | |
| 1777 | 2018 Nov. 1T18:02:48 | NO | | | NO | |
| 1777 | 2018 Nov. 2T18:26:38 | YES | MILD | | YES | 1 |
| 1777 | 2018 Nov. 3T18:00:31 | NO | | | NO | |
| 1777 | 2018 Nov. 4T18:00:02 | YES | MILD | | YES | 1 |
| 1777 | 2018 Nov. 5T18:00:47 | NO | | | NO | |
| 1777 | 2018 Nov. 6T18:00:35 | NO | | | NO | |
| 1777 | 2018 Nov. 7T18:07:55 | NO | | | NO | |
| 1777 | 2018 Nov. 8T18:00:37 | YES | MODERATE | | YES | 1 |
| 1777 | 2018 Nov. 9T18:00:30 | NO | | | NO | |
| 1777 | 2018 Nov. 10T19:17:39 | YES | MODERATE | | YES | 1 |
| 1777 | 2018 Nov. 11T21:01:24 | NO | | | NO | |
| 1777 | 2018 Nov. 12T19:34:02 | NO | | | NO | |
| 1777 | 2018 Nov. 13T18:00:01 | YES | SEVERE | | YES | 1 |
| 1777 | 2018 Nov. 14T18:00:34 | NO | | | NO | |
| 1777 | 2018 Nov. 15T18:00:31 | YES | MILD | | YES | 1 |
| 1777 | 2018 Nov. 16T22:16:05 | NO | | | NO | |
| 1777 | 2018 Nov. 17T18:00:31 | YES | MILD | | YES | 1 |
| 1777 | 2018 Nov. 18T18:08:52 | NO | | | NO | |
| 1777 | 2018 Nov. 19T20:44:51 | YES | MILD | | YES | 1 |
| 1777 | 2018 Nov. 20T18:00:33 | NO | | | NO | |
| 1777 | 2018 Nov. 21T18:00:52 | YES | MILD | | NO | |
| 1777 | 2018 Nov. 22T18:00:36 | NO | | | NO | |
| 1777 | 2018 Nov. 23T18:00:36 | NO | | | NO | |
| 1777 | 2018 Nov. 24T18:00:39 | NO | | | NO | |
| 1777 | 2018 Nov. 25T18:00:41 | YES | MILD | | YES | 1 |
| 1777 | 2018 Nov. 26T18:27:14 | NO | | | NO | |
| 1777 | 2018 Nov. 27T18:00:47 | YES | MILD | | YES | 1 |
| 1777 | 2018 Nov. 28T18:00:02 | NO | | | NO | |
| 1777 | 2018 Nov. 29T18:00:01 | YES | MILD | | YES | 1 |
| 1777 | 2018 Nov. 30T18:00:55 | NO | | | NO | |
| 1777 | 2018 Dec. 1T18:00:37 | NO | | | NO | |
| 1777 | 2018 Dec. 2T18:00:35 | YES | MODERATE | | YES | 1 |
| 1777 | 2018 Dec. 3T18:00:33 | NO | | | NO | |
| 1777 | 2018 Dec. 4T18:00:50 | YES | MILD | | YES | 1 |
| 1777 | 2018 Dec. 5T18:00:03 | NO | | | NO | |
| 1777 | 2018 Dec. 6T18:00:37 | YES | MODERATE | | YES | 1 |
| 1777 | 2018 Dec. 7T18:00:01 | NO | | | NO | |
| 1777 | 2018 Dec. 8T18:59:10 | YES | MILD | | YES | 1 |
| 1777 | 2018 Dec. 9T18:01:40 | NO | | | NO | |
| 1777 | 2018 Dec. 10T18:00:01 | YES | MILD | | YES | 1 |
| 1777 | 2018 Dec. 11T18:00:33 | NO | | | NO | |
| 1777 | 2018 Dec. 12T18:00:29 | NO | | | NO | |
| 1777 | 2018 Dec. 13T18:00:23 | YES | MILD | | YES | 1 |
| 1777 | 2018 Dec. 14T18:00:03 | NO | | | NO | |
| 1777 | 2018 Dec. 15T19:36:26 | YES | MILD | | YES | 1 |
| 1777 | 2018 Dec. 16T18:00:31 | NO | | | NO | |
| 1777 | 2018 Dec. 17T18:02:45 | NO | | | NO | |
| 1777 | 2018 Dec. 18T18:00:01 | NO | | | NO | |
| 1777 | 2018 Dec. 19T18:01:08 | NO | | | NO | |
| 1777 | 2018 Dec. 20T18:00:43 | YES | MODERATE | | YES | 1 |
| 1777 | 2018 Dec. 21T18:00:31 | NO | | | NO | |
| 1777 | 2018 Dec. 22T20:59:03 | YES | MILD | | YES | 1 |
| 1777 | 2018 Dec. 23T18:22:04 | NO | | | NO | |
| 1777 | 2018 Dec. 24T18:00:29 | NO | | | NO | |
| 1777 | 2018 Dec. 25T20:36:19 | NO | | | NO | |
| 1777 | 2018 Dec. 26T18:00:54 | NO | | | NO | |
| 1777 | 2018 Dec. 27T18:00:03 | YES | MILD | | YES | 1 |
| 1777 | 2018 Dec. 28T18:00:41 | NO | | | NO | |
| 1777 | 2018 Dec. 29T18:00:49 | YES | MODERATE | | YES | 1 |
| 1777 | 2018 Dec. 30T18:00:44 | NO | | | NO | |
| 1777 | 2018 Dec. 31T18:00:03 | NO | | | NO | |
| 1777 | 2019 Jan. 1T18:00:38 | NO | | | NO | |
| 1777 | 2019 Jan. 2T18:00:30 | YES | MILD | | YES | 1 |
| 1777 | 2019 Jan. 3T18:00:41 | NO | | | NO | |
| 1777 | 2019 Jan. 4T18:00:43 | NO | | | NO | |
| 1777 | 2019 Jan. 5T18:00:44 | YES | SEVERE | | YES | 1 |
| 1777 | 2019 Jan. 6T18:00:48 | NO | | | NO | |
| 1777 | 2019 Jan. 7T18:00:42 | YES | MILD | | YES | 1 |
| 1777 | 2019 Jan. 8T18:00:41 | NO | | | NO | |
| 1777 | 2019 Jan. 9T18:00:33 | NO | | | NO | |
| 1777 | 2019 Jan. 10T18:00:02 | NO | | | NO | |
| 1777 | 2019 Jan. 11T18:00:30 | YES | MILD | | YES | 1 |
| 1777 | 2019 Jan. 12T18:01:27 | NO | | | NO | |
| 1777 | 2019 Jan. 13T18:00:36 | YES | SEVERE | | YES | 1 |
| 1777 | 2019 Jan. 14T18:00:27 | YES | MILD | | YES | 1 |
| 1777 | 2019 Jan. 15T18:00:36 | NO | | | NO | |
| 1777 | 2019 Jan. 16T18:00:01 | NO | | | NO | |
| 1777 | 2019 Jan. 17T18:00:33 | NO | | | NO | |
| 1777 | 2019 Jan. 18T18:00:01 | NO | | | NO | |
| 1777 | 2019 Jan. 19T18:00:08 | YES | MILD | | YES | 1 |
| 1777 | 2019 Jan. 20T18:00:31 | NO | | | NO | |
| 1777 | 2019 Jan. 21T18:00:33 | YES | SEVERE | | YES | 1 |
| 1777 | 2019 Jan. 22T18:00:50 | NO | | | NO | |
| 1777 | 2019 Jan. 23T18:00:27 | NO | | | NO | |
| 1777 | 2019 Jan. 24T18:00:41 | NO | | | NO | |
| 1777 | 2019 Jan. 25T18:00:43 | NO | | | NO | |
| 1777 | 2019 Jan. 26T18:00:23 | YES | MILD | | YES | 1 |
| 1777 | 2019 Jan. 27T18:01:26 | NO | | | NO | |
| 1777 | 2019 Jan. 28T18:01:05 | NO | | | NO | |
| 1777 | 2019 Jan. 29T18:00:01 | NO | | | NO | |
| 1777 | 2019 Jan. 30T18:00:33 | YES | MILD | | NO | |
| 1777 | 2019 Jan. 31T20:33:50 | NO | | | NO | |
| 1777 | 2019 Feb. 1T18:00:37 | YES | MODERATE | | YES | 1 |
| 1777 | 2019 Feb. 2T18:41:55 | NO | | | NO | |
| 1777 | 2019 Feb. 3T18:01:17 | NO | | | NO | |
| 1777 | 2019 Feb. 4T18:00:27 | YES | MODERATE | | YES | 1 |
| 1777 | 2019 Feb. 5T18:00:40 | YES | MILD | | NO | |
| 1777 | 2019 Feb. 6T18:01:04 | YES | MILD | | YES | 1 |
| 1777 | 2019 Feb. 07T18:00:35 | NO | | | NO | |
| 1777 | 2019 Feb. 8T18:00:46 | NO | | | NO | |
| 1777 | 2019 Feb. 9T18:00:33 | YES | MODERATE | | YES | 1 |
| 1777 | 2019 Feb. 10T18:02:32 | NO | | | NO | |
| 1777 | 2019 Feb. 11T18:00:33 | YES | MODERATE | | YES | 1 |
| 1777 | 2019 Feb. 12T18:00:37 | NO | | | NO | |
| 1777 | 2019 Feb. 13T18:00:32 | NO | | | NO | |
| 1777 | 2019 Feb. 14T18:00:25 | NO | | | NO | |
| 1777 | 2019 Feb. 15T18:00:30 | YES | MILD | | YES | 1 |
| 1777 | 2019 Feb. 16T21:36:16 | NO | | | NO | |
| 1777 | 2019 Feb. 17T18:00:12 | YES | MILD | | YES | 1 |
| 1777 | 2019 Feb. 18T18:00:27 | NO | | | NO | |
| 1777 | 2019 Feb. 19T18:00:39 | YES | MILD | | YES | 1 |
| 1777 | 2019 Feb. 20T18:00:27 | NO | | | NO | |
| 1777 | 2019 Feb. 21T18:00:32 | NO | | | NO | |
| 1777 | 2019 Feb. 22T18:00:33 | YES | MILD | | YES | 1 |
| 1777 | 2019 Feb. 23T18:00:42 | NO | | | NO | |
| 1777 | 2019 Feb. 24T18:13:12 | NO | | | NO | |
| 1777 | 2019 Feb. 25T18:00:39 | NO | | | NO | |
| 1777 | 2019 Feb. 26T18:00:42 | NO | | | NO | |
| 1777 | 2019 Feb. 27T19:00:49 | YES | MILD | | YES | 1 |
| 1777 | 2019 Feb. 28T18:00:32 | NO | | | NO | |
| 1777 | 2019 Mar. 1T18:00:34 | NO | | | NO | |
| 1777 | 2019 Mar. 2T18:00:04 | YES | MILD | | YES | 1 |
| 1777 | 2019 Mar. 3T18:00:18 | NO | | | NO | |
| 1777 | 2019 Mar. 4T18:00:36 | YES | MILD | | YES | 1 |
| 1777 | 2019 Mar. 5T18:00:36 | NO | | | NO | |
| 1777 | 2019 Mar. 6T18:00:28 | YES | MODERATE | | YES | 1 |
| 1777 | 2019 Mar. 7T18:00:30 | NO | | | NO | |
| 1777 | 2019 Mar. 8T19:13:43 | NO | | | NO | |
| 1777 | 2019 Mar. 9T18:00:33 | NO | | | NO | |
| 1777 | 2019 Mar. 10T18:00:33 | YES | MILD | | YES | 1 |
| 1777 | 2019 Mar. 11T18:00:40 | NO | | | NO | |
| 1777 | 2019 Mar. 12T18:00:30 | YES | MILD | | NO | |
| 1777 | 2019 Mar. 13T19:34:07 | NO | | | NO | |
| 1777 | 2019 Mar. 14T18:00:38 | YES | MILD | | YES | 1 |
| 1777 | 2019 Mar. 15T18:00:27 | NO | | | NO | |
| 1777 | 2019 Mar. 16T19:51:15 | YES | MODERATE | | YES | 1 |
| 1777 | 2019 Mar. 17T18:00:36 | YES | MILD | | NO | |
| 1777 | 2019 Mar. 18T18:00:34 | YES | MILD | | YES | 1 |
| 1777 | 2019 Mar. 19T18:15:34 | NO | | | NO | |
| 1777 | 2019 Mar. 20T18:00:34 | NO | | | NO | |
| 1777 | 2019 Mar. 21T18:00:38 | YES | MILD | | YES | 1 |
| 1777 | 2019 Mar. 22T18:00:40 | YES | MODERATE | | YES | 1 |
| 1777 | 2019 Mar. 23T18:00:33 | NO | | | NO | |
| 1777 | 2019 Mar. 24T19:50:22 | NO | | | NO | |
| 1777 | 2019 Mar. 25T18:01:26 | YES | MILD | | YES | 1 |
| 1777 | 2019 Mar. 26T18:00:10 | NO | | | NO | |
| 1777 | 2019 Mar. 27T09:12:43 | YES | MODERATE | | YES | 1 |

Subject 1777 is a 56 year-old Caucasian female with history of 9-14 migraine attacks (without aura) per month since the age of 27. Past medication used to treat her headache has been zolmitriptan, rizatriptan, excedrin, ibuprofen, and naproxene. Additionally, she has been receiving Botox® injections every three months from December 2017.

TABLE 18

| | | Did you | | Take other | Take study | |
|---|---|---|---|---|---|---|
| | | have a | | med | medication | Number |
| | | migraine | | [Observation | [Treatment | of |
| Subject | Date/Time of Finding | headache? | Severity | phase] | phase] | tablets |
| 1283 | 2017 Dec. 6T19:31:22 | NO | | | | |
| 1283 | 2017 Dec. 7T18:50:14 | YES | MODERATE | YES | | |
| 1283 | 2017 Dec. 8T19:35:03 | YES | MODERATE | YES | | |
| 1283 | 2017 Dec. 9T22:29:14 | NO | | | | |
| 1283 | 2017 Dec. 10T19:54:00 | NO | | | | |
| 1283 | 2017 Dec. 12T18:45:12 | NO | | | | |
| 1283 | 2017 Dec. 13T20:04:18 | NO | | | | |
| 1283 | 2017 Dec. 14T19:30:30 | NO | | | | |
| 1283 | 2017 Dec. 15T23:06:51 | YES | MODERATE | YES | | |
| 1283 | 2017 Dec. 16T18:16:12 | YES | MILD | NO | | |
| 1283 | 2017 Dec. 17T18:12:41 | YES | MODERATE | YES | | |
| 1283 | 2017 Dec. 18T19:31:58 | NO | | | | |
| 1283 | 2017 Dec. 19T18:32:31 | NO | | | | |
| 1283 | 2017 Dec. 20T18:32:12 | NO | | | | |
| 1283 | 2017 Dec. 21T20:43:27 | NO | | | | |
| 1283 | 2017 Dec. 22T19:21:51 | YES | MILD | YES | | |
| 1283 | 2017 Dec. 23T18:58:21 | NO | | | | |
| 1283 | 2017 Dec. 24T19:30:36 | YES | MODERATE | YES | | |
| 1283 | 2017 Dec. 25T19:30:38 | NO | | | | |
| 1283 | 2017 Dec. 26T18:16:27 | YES | MILD | YES | | |
| 1283 | 2017 Dec. 27T18:58:59 | NO | | | | |
| 1283 | 2017 Dec. 28T19:50:25 | NO | | | | |
| 1283 | 2017 Dec. 29T18:00:53 | NO | | | | |
| 1283 | 2017 Dec. 30T19:31:49 | NO | | | | |
| 1283 | 2017 Dec. 31T18:58:07 | NO | | | | |
| 1283 | 2018 Jan. 1T18:01:06 | YES | MODERATE | YES | | |
| 1283 | 2018 Jan. 2T18:13:03 | YES | MILD | YES | | |
| 1283 | 2018 Jan. 3T19:19:16 | YES | SEVERE | YES | | |
| 1283 | 2018 Jan. 4T21:38:05 | NO | | | | |
| 1283 | 2018 Jan. 5T19:04:47 | NO | | | NO | |
| 1283 | 2018 Jan. 6T18:38:40 | NO | | | NO | |
| 1283 | 2018 Jan. 7T19:30:34 | NO | | | NO | |
| 1283 | 2018 Jan. 8T18:47:07 | NO | | | NO | |
| 1283 | 2018 Jan. 9T18:28:19 | NO | | | NO | |
| 1283 | 2018 Jan. 10T19:00:19 | NO | | | NO | |
| 1283 | 2018 Jan. 11T18:14:42 | YES | MODERATE | | YES | 1 |
| 1283 | 2018 Jan. 12T22:15:03 | NO | | | NO | |
| 1283 | 2018 Jan. 13T18:01:58 | YES | MODERATE | | YES | 1 |
| 1283 | 2018 Jan. 14T18:18:12 | NO | | | NO | |
| 1283 | 2018 Jan. 15T18:26:56 | NO | | | NO | |
| 1283 | 2018 Jan. 16T19:30:38 | NO | | | NO | |
| 1283 | 2018 Jan. 17T19:10:14 | NO | | | NO | |
| 1283 | 2018 Jan. 18T18:22:25 | YES | MODERATE | | YES | 1 |
| 1283 | 2018 Jan. 19T19:32:02 | NO | | | NO | |
| 1283 | 2018 Jan. 20T18:57:30 | NO | | | NO | |
| 1283 | 2018 Jan. 21T18:27:50 | NO | | | NO | |
| 1283 | 2018 Jan. 22T18:06:37 | NO | | | NO | |
| 1283 | 2018 Jan. 23T18:06:47 | NO | | | NO | |
| 1283 | 2018 Jan. 24T19:30:37 | YES | MODERATE | | YES | 1 |
| 1283 | 2018 Jan. 25T18:20:28 | NO | | | NO | |
| 1283 | 2018 Jan. 26T19:32:23 | NO | | | NO | |
| 1283 | 2018 Jan. 27T19:24:08 | YES | MILD | | YES | 1 |
| 1283 | 2018 Jan. 28T19:30:40 | NO | | | NO | |
| 1283 | 2018 Jan. 29T18:23:50 | NO | | | NO | |
| 1283 | 2018 Jan. 30T19:23:58 | NO | | | NO | |
| 1283 | 2018 Jan. 31T18:16:12 | NO | | | NO | |
| 1283 | 2018 Feb. 1T18:04:21 | YES | MILD | | YES | 1 |
| 1283 | 2018 Feb. 2T19:49:05 | NO | | | NO | |
| 1283 | 2018 Feb. 3T22:45:07 | YES | MILD | | YES | 1 |
| 1283 | 2018 Feb. 4T20:44:53 | NO | | | NO | |
| 1283 | 2018 Feb. 5T18:41:37 | NO | | | NO | |
| 1283 | 2018 Feb. 6T18:35:38 | NO | | | NO | |
| 1283 | 2018 Feb. 7T18:11:29 | NO | | | NO | |
| 1283 | 2018 Feb. 8T19:31:53 | YES | MODERATE | | YES | 1 |
| 1283 | 2018 Feb. 9T20:05:58 | NO | | | NO | |
| 1283 | 2018 Feb. 10T19:30:46 | YES | MODERATE | | YES | 1 |
| 1283 | 2018 Feb. 11T19:09:44 | NO | | | NO | |
| 1283 | 2018 Feb. 12T18:53:25 | NO | | | NO | |
| 1283 | 2018 Feb. 13T21:45:31 | NO | | | NO | |
| 1283 | 2018 Feb. 14T18:06:22 | YES | MILD | | NO | |
| 1283 | 2018 Feb. 15T19:30:32 | YES | MODERATE | | YES | 1 |
| 1283 | 2018 Feb. 16T19:10:28 | NO | | | NO | |
| 1283 | 2018 Feb. 17T19:22:31 | NO | | | NO | |
| 1283 | 2018 Feb. 18T19:30:31 | NO | | | NO | |
| 1283 | 2018 Feb. 19T19:07:18 | NO | | | NO | |
| 1283 | 2018 Feb. 20T19:58:13 | NO | | | NO | |
| 1283 | 2018 Feb. 21T18:59:06 | NO | | | NO | |
| 1283 | 2018 Feb. 22T19:30:36 | YES | SEVERE | | YES | 1 |
| 1283 | 2018 Feb. 23T20:58:05 | NO | | | NO | |
| 1283 | 2018 Feb. 24T18:50:33 | YES | MODERATE | | YES | 1 |
| 1283 | 2018 Feb. 25T18:06:01 | NO | | | NO | |
| 1283 | 2018 Feb. 26T19:28:45 | NO | | | NO | |
| 1283 | 2018 Feb. 27T18:29:08 | NO | | | NO | |
| 1283 | 2018 Feb. 28T18:10:07 | NO | | | NO | |
| 1283 | 2018 Mar. 1T19:44:41 | NO | | | NO | |
| 1283 | 2018 Mar. 2T19:44:05 | YES | SEVERE | | YES | 1 |
| 1283 | 2018 Mar. 3T18:28:11 | YES | MILD | | YES | 1 |
| 1283 | 2018 Mar. 4T18:10:58 | NO | | | NO | |
| 1283 | 2018 Mar. 5T19:30:31 | NO | | | NO | |
| 1283 | 2018 Mar. 6T19:30:25 | NO | | | NO | |
| 1283 | 2018 Mar. 7T19:32:34 | NO | | | NO | |
| 1283 | 2018 Mar. 8T19:35:24 | NO | | | NO | |
| 1283 | 2018 Mar. 9T19:30:46 | NO | | | NO | |
| 1283 | 2018 Mar. 10T18:02:53 | NO | | | NO | |
| 1283 | 2018 Mar. 11T19:30:48 | YES | MILD | | YES | 1 |
| 1283 | 2018 Mar. 12T19:23:51 | NO | | | NO | |
| 1283 | 2018 Mar. 13T18:47:56 | NO | | | NO | |
| 1283 | 2018 Mar. 14T19:06:27 | NO | | | NO | |
| 1283 | 2018 Mar. 15T19:18:49 | NO | | | NO | |
| 1283 | 2018 Mar. 16T18:39:50 | YES | MILD | | YES | 1 |
| 1283 | 2018 Mar. 17T19:30:36 | NO | | | NO | |
| 1283 | 2018 Mar. 18T18:18:52 | YES | MILD | | YES | 1 |
| 1283 | 2018 Mar. 19T18:41:45 | NO | | | NO | |
| 1283 | 2018 Mar. 20T18:47:24 | NO | | | NO | |
| 1283 | 2018 Mar. 21T18:50:27 | NO | | | NO | |
| 1283 | 2018 Mar. 22T18:34:54 | YES | MODERATE | | YES | 1 |
| 1283 | 2018 Mar. 23T19:12:33 | NO | | | NO | |
| 1283 | 2018 Mar. 24T18:04:54 | YES | MODERATE | | YES | 1 |
| 1283 | 2018 Mar. 25T19:39:07 | NO | | | NO | |
| 1283 | 2018 Mar. 26T19:31:41 | NO | | | NO | |
| 1283 | 2018 Mar. 27T19:15:54 | NO | | | NO | |
| 1283 | 2018 Mar. 28T19:09:27 | NO | | | NO | |
| 1283 | 2018 Mar. 29T20:23:57 | YES | MILD | | YES | 1 |
| 1283 | 2018 Mar. 30T18:14:16 | NO | | | NO | |
| 1283 | 2018 Mar. 31T18:27:32 | NO | | | NO | |
| 1283 | 2018 Apr. 1T19:13:24 | NO | | | NO | |
| 1283 | 2018 Apr. 2T19:33:03 | NO | | | NO | |
| 1283 | 2018 Apr. 3T18:43:57 | NO | | | NO | |
| 1283 | 2018 Apr. 4T19:19:46 | NO | | | NO | |
| 1283 | 2018 Apr. 5T18:56:10 | YES | MODERATE | | YES | 1 |
| 1283 | 2018 Apr. 6T19:30:23 | NO | | | NO | |
| 1283 | 2018 Apr. 7T20:41:56 | YES | MILD | | YES | 1 |
| 1283 | 2018 Apr. 8T18:11:30 | NO | | | NO | |
| 1283 | 2018 Apr. 9T18:15:00 | NO | | | NO | |
| 1283 | 2018 Apr. 10T19:30:30 | NO | | | NO | |
| 1283 | 2018 Apr. 11T20:13:39 | NO | | | NO | |
| 1283 | 2018 Apr. 12T18:35:53 | YES | MILD | | YES | 1 |
| 1283 | 2018 Apr. 13T20:58:17 | NO | | | NO | |
| 1283 | 2018 Apr. 14T19:34:19 | YES | MILD | | YES | 1 |
| 1283 | 2018 Apr. 15T19:36:20 | NO | | | NO | |
| 1283 | 2018 Apr. 16T19:05:07 | NO | | | NO | |
| 1283 | 2018 Apr. 17T21:40:34 | NO | | | NO | |
| 1283 | 2018 Apr. 18T18:27:56 | NO | | | NO | |
| 1283 | 2018 Apr. 19T20:43:59 | YES | MODERATE | | YES | 1 |
| 1283 | 2018 Apr. 20T19:30:29 | NO | | | NO | |
| 1283 | 2018 Apr. 21T18:28:13 | NO | | | NO | |
| 1283 | 2018 Apr. 22T18:23:09 | NO | | | NO | |
| 1283 | 2018 Apr. 23T18:48:56 | NO | | | NO | |
| 1283 | 2018 Apr. 24T21:54:15 | NO | | | NO | |
| 1283 | 2018 Apr. 25T19:32:33 | NO | | | NO | |
| 1283 | 2018 Apr. 26T19:30:34 | YES | MODERATE | | YES | 1 |
| 1283 | 2018 Apr. 27T19:11:30 | NO | | | NO | |
| 1283 | 2018 Apr. 28T23:03:44 | YES | MODERATE | | YES | 1 |
| 1283 | 2018 Apr. 29T18:28:54 | NO | | | NO | |
| 1283 | 2018 Apr. 30T19:32:53 | NO | | | NO | |
| 1283 | 2018 May 1T21:49:05 | NO | | | NO | |
| 1283 | 2018 May 2T20:34:25 | NO | | | NO | |
| 1283 | 2018 May 3T19:32:27 | YES | MILD | | YES | 1 |
| 1283 | 2018 May 4T18:51:34 | NO | | | NO | |
| 1283 | 2018 May 5T19:31:17 | NO | | | NO | |
| 1283 | 2018 May 6T18:05:52 | NO | | | NO | |
| 1283 | 2018 May 7T19:10:13 | YES | MILD | | YES | 1 |
| 1283 | 2018 May 8T18:23:39 | NO | | | NO | |
| 1283 | 2018 May 9T18:31:30 | NO | | | NO | |
| 1283 | 2018 May 10T19:31:25 | YES | MILD | | YES | 1 |
| 1283 | 2018 May 11T22:33:43 | NO | | | NO | |
| 1283 | 2018 May 12T21:30:30 | NO | | | NO | |
| 1283 | 2018 May 13T20:02:29 | NO | | | NO | |
| 1283 | 2018 May 14T20:00:39 | NO | | | NO | |
| 1283 | 2018 May 15T19:14:34 | NO | | | NO | |
| 1283 | 2018 May 16T19:12:03 | NO | | | NO | |
| 1283 | 2018 May 17T18:50:04 | NO | | | NO | |
| 1283 | 2018 May 18T19:45:07 | NO | | | NO | |
| 1283 | 2018 May 19T21:59:38 | YES | MILD | | YES | 1 |
| 1283 | 2018 May 20T20:01:35 | NO | | | NO | |
| 1283 | 2018 May 21T18:08:27 | NO | | | NO | |
| 1283 | 2018 May 22T18:29:21 | NO | | | NO | |
| 1283 | 2018 May 23T19:04:57 | NO | | | NO | |
| 1283 | 2018 May 24T19:31:22 | NO | | | NO | |
| 1283 | 2018 May 27T19:45:43 | YES | MILD | | YES | 1 |
| 1283 | 2018 May 28T19:30:51 | NO | | | NO | |
| 1283 | 2018 May 29T18:20:57 | NO | | | NO | |
| 1283 | 2018 May 30T19:25:43 | NO | | | NO | |
| 1283 | 2018 May 31T19:47:01 | NO | | | NO | |
| 1283 | 2018 Jun. 1T19:35:51 | YES | MILD | | YES | 1 |
| 1283 | 2018 Jun. 2T20:15:23 | NO | | | NO | |
| 1283 | 2018 Jun. 3T18:35:36 | YES | MILD | | YES | 1 |
| 1283 | 2018 Jun. 4T19:32:56 | NO | | | NO | |
| 1283 | 2018 Jun. 5T21:25:26 | NO | | | NO | |
| 1283 | 2018 Jun. 6T18:11:56 | NO | | | NO | |
| 1283 | 2018 Jun. 7T19:30:30 | NO | | | NO | |
| 1283 | 2018 Jun. 8T18:20:32 | YES | MILD | | YES | 1 |
| 1283 | 2018 Jun. 9T18:32:05 | NO | | | NO | |
| 1283 | 2018 Jun. 10T18:41:54 | NO | | | NO | |
| 1283 | 2018 Jun. 11T18:38:03 | NO | | | NO | |
| 1283 | 2018 Jun. 12T18:19:32 | NO | | | NO | |
| 1283 | 2018 Jun. 13T19:32:26 | NO | | | NO | |
| 1283 | 2018 Jun. 14T18:05:49 | NO | | | NO | |
| 1283 | 2018 Jun. 15T18:30:09 | YES | MILD | | YES | 1 |
| 1283 | 2018 Jun. 16T18:01:33 | NO | | | NO | |
| 1283 | 2018 Jun. 17T19:31:41 | NO | | | NO | |
| 1283 | 2018 Jun. 18T18:15:48 | YES | MILD | | YES | 1 |
| 1283 | 2018 Jun. 19T18:07:43 | NO | | | NO | |
| 1283 | 2018 Jun. 20T19:11:15 | NO | | | NO | |
| 1283 | 2018 Jun. 21T19:34:13 | NO | | | NO | |
| 1283 | 2018 Jun. 22T19:53:23 | NO | | | NO | |
| 1283 | 2018 Jun. 23T19:30:38 | YES | MILD | | YES | 1 |
| 1283 | 2018 Jun. 24T18:02:13 | NO | | | NO | |
| 1283 | 2018 Jun. 25T20:16:31 | NO | | | NO | |
| 1283 | 2018 Jun. 26T18:05:24 | NO | | | NO | |
| 1283 | 2018 Jun. 27T19:31:23 | NO | | | NO | |
| 1283 | 2018 Jun. 28T19:11:17 | NO | | | NO | |
| 1283 | 2018 Jun. 29T21:45:50 | YES | MILD | | YES | 1 |
| 1283 | 2018 Jun. 30T22:04:27 | NO | | | NO | |
| 1283 | 2018 Jul. 1T18:01:03 | YES | MILD | | YES | 1 |
| 1283 | 2018 Jul. 2T18:43:52 | NO | | | NO | |
| 1283 | 2018 Jul. 3T18:25:29 | NO | | | NO | |
| 1283 | 2018 Jul. 4T21:46:00 | NO | | | NO | |
| 1283 | 2018 Jul. 5T21:03:28 | YES | MILD | | YES | 1 |
| 1283 | 2018 Jul. 6T19:35:37 | NO | | | NO | |
| 1283 | 2018 Jul. 7T22:18:02 | NO | | | NO | |
| 1283 | 2018 Jul. 8T18:17:19 | NO | | | NO | |
| 1283 | 2018 Jul. 9T18:01:30 | NO | | | NO | |
| 1283 | 2018 Jul. 10T18:15:06 | NO | | | NO | |
| 1283 | 2018 Jul. 12T21:45:44 | NO | | | NO | |
| 1283 | 2018 Jul. 13T19:33:33 | YES | MILD | | YES | 1 |
| 1283 | 2018 Jul. 14T21:37:12 | NO | | | NO | |
| 1283 | 2018 Jul. 15T19:30:33 | NO | | | NO | |
| 1283 | 2018 Jul. 16T18:52:43 | NO | | | NO | |
| 1283 | 2018 Jul. 17T18:58:16 | NO | | | NO | |
| 1283 | 2018 Jul. 18T21:46:54 | NO | | | NO | |
| 1283 | 2018 Jul. 19T19:34:47 | NO | | | NO | |
| 1283 | 2018 Jul. 20T18:56:48 | YES | MODERATE | | YES | 1 |
| 1283 | 2018 Jul. 21T18:12:21 | NO | | | NO | |
| 1283 | 2018 Jul. 22T20:36:42 | NO | | | NO | |
| 1283 | 2018 Jul. 23T22:51:50 | NO | | | NO | |
| 1283 | 2018 Jul. 24T22:41:42 | NO | | | NO | |
| 1283 | 2018 Jul. 25T19:54:11 | NO | | | NO | |
| 1283 | 2018 Jul. 26T19:35:41 | YES | MILD | | YES | 1 |
| 1283 | 2018 Jul. 27T19:45:29 | NO | | | NO | |
| 1283 | 2018 Jul. 28T18:05:49 | YES | MILD | | YES | 1 |
| 1283 | 2018 Jul. 29T19:31:27 | NO | | | NO | |
| 1283 | 2018 Jul. 30T19:36:11 | NO | | | NO | |
| 1283 | 2018 Jul. 31T19:32:09 | NO | | | NO | |
| 1283 | 2018 Aug. 1T21:22:07 | YES | MODERATE | | YES | 1 |
| 1283 | 2018 Aug. 2T20:18:32 | NO | | | NO | |
| 1283 | 2018 Aug. 3T20:01:08 | NO | | | NO | |
| 1283 | 2018 Aug. 4T21:59:40 | NO | | | NO | |
| 1283 | 2018 Aug. 5T18:07:04 | YES | MILD | | YES | 1 |
| 1283 | 2018 Aug. 6T18:12:20 | NO | | | NO | |
| 1283 | 2018 Aug. 7T21:00:51 | NO | | | NO | |
| 1283 | 2018 Aug. 8T18:57:16 | YES | MILD | | YES | 1 |
| 1283 | 2018 Aug. 9T19:27:40 | NO | | | NO | |
| 1283 | 2018 Aug. 10T19:36:05 | NO | | | NO | |
| 1283 | 2018 Aug. 11T23:30:13 | YES | MILD | | YES | 1 |
| 1283 | 2018 Aug. 12T19:31:28 | NO | | | NO | |
| 1283 | 2018 Aug. 13T19:30:26 | NO | | | NO | |
| 1283 | 2018 Aug. 14T19:30:49 | NO | | | NO | |

Subject 1283 is a 52 year-old Caucasian female with history of 2-8 attacks (without aura) per month since the age of 16. Past medication used to treat her headache has been eletriptan and fioricet. Additionally, she has been receiving Botox® injections every three months from April 2015.

TABLE 19

| | | | | Take other | Take study | |
|---|---|---|---|---|---|---|
| | | Did you have | | med | medication | |
| | | a migraine | | [Observation | [Treatment | Number of |
| Subject | Date/Time of Finding | headache? | Severity | phase] | phase] | tablets |
| 2150 | 2018 Apr. 19T22:16:22 | YES | MILD | NO | | |
| 2150 | 2018 Apr. 20T22:00:35 | YES | MILD | NO | | |
| 2150 | 2018 Apr. 21T18:15:06 | NO | | | | |
| 2150 | 2018 Apr. 22T23:53:45 | NO | | | | |
| 2150 | 2018 Apr. 23T19:28:51 | YES | MODERATE | NO | | |
| 2150 | 2018 Apr. 24T20:31:58 | NO | | | | |
| 2150 | 2018 Apr. 25T21:01:46 | NO | | | | |
| 2150 | 2018 Apr. 26T22:15:47 | NO | | | | |
| 2150 | 2018 Apr. 27T19:40:21 | YES | MILD | NO | | |
| 2150 | 2018 Apr. 30T18:01:20 | YES | MODERATE | YES | | |
| 2150 | 2018 May 1T18:45:35 | NO | | | | |
| 2150 | 2018 May 2T20:25:01 | NO | | | | |
| 2150 | 2018 May 3T18:00:28 | NO | | | | |
| 2150 | 2018 May 4T18:03:23 | YES | MODERATE | NO | | |
| 2150 | 2018 May 5T19:13:44 | NO | | | | |
| 2150 | 2018 May 6T23:08:02 | NO | | | | |
| 2150 | 2018 May 7T20:29:31 | YES | MODERATE | NO | | |
| 2150 | 2018 May 8T22:00:31 | YES | MODERATE | NO | | |
| 2150 | 2018 May 9T20:36:43 | YES | MILD | NO | | |
| 2150 | 2018 May 10T19:34:43 | NO | | | | |
| 2150 | 2018 May 11T18:01:19 | NO | | | | |
| 2150 | 2018 May 12T20:11:01 | NO | | | | |
| 2150 | 2018 May 13T18:29:53 | YES | MODERATE | NO | | |
| 2150 | 2018 May 14T22:10:11 | NO | | | | |
| 2150 | 2018 May 16T18:01:10 | NO | | | | |
| 2150 | 2018 May 17T18:00:16 | NO | | | | |
| 2150 | 2018 May 18T18:08:38 | NO | | | | |
| 2150 | 2018 May 19T18:00:46 | NO | | | | |
| 2150 | 2018 May 20T18:07:15 | YES | MODERATE | YES | | |
| 2150 | 2018 May 21T22:22:54 | NO | | | NO | |
| 2150 | 2018 May 22T22:01:01 | NO | | | NO | |
| 2150 | 2018 May 24T20:04:46 | NO | | | NO | |
| 2150 | 2018 May 25T18:01:09 | NO | | | NO | |
| 2150 | 2018 May 26T23:55:26 | YES | SEVERE | | YES | 1 |
| 2150 | 2018 May 27T18:58:23 | NO | | | NO | |
| 2150 | 2018 May 28T20:22:27 | YES | MILD | | YES | 1 |
| 2150 | 2018 May 29T20:47:26 | NO | | | NO | |
| 2150 | 2018 May 30T22:06:39 | YES | MILD | | YES | 1 |
| 2150 | 2018 May 31T22:15:19 | NO | | | NO | |
| 2150 | 2018 Jun. 1T22:00:32 | NO | | | NO | |
| 2150 | 2018 Jun. 2T22:00:28 | NO | | | NO | |
| 2150 | 2018 Jun. 3T18:04:50 | NO | | | NO | |
| 2150 | 2018 Jun. 4T20:16:22 | YES | MILD | | YES | 1 |
| 2150 | 2018 Jun. 5T22:05:30 | NO | | | NO | |
| 2150 | 2018 Jun. 6T21:41:32 | YES | MODERATE | | YES | 1 |
| 2150 | 2018 Jun. 7T22:00:33 | YES | MODERATE | | YES | 1 |
| 2150 | 2018 Jun. 8T19:46:21 | YES | MODERATE | | YES | 1 |
| 2150 | 2018 Jun. 9T19:01:15 | NO | | | NO | |
| 2150 | 2018 Jun. 10T21:32:43 | NO | | | NO | |
| 2150 | 2018 Jun. 11T21:26:42 | NO | | | NO | |
| 2150 | 2018 Jun. 12T19:34:02 | NO | | | NO | |
| 2150 | 2018 Jun. 13T19:28:15 | YES | MILD | | NO | |
| 2150 | 2018 Jun. 14T18:01:36 | NO | | | NO | |
| 2150 | 2018 Jun. 15T18:17:15 | YES | MILD | | YES | 1 |
| 2150 | 2018 Jun. 16T19:21:13 | YES | MODERATE | | YES | 1 |
| 2150 | 2018 Jun. 17T22:45:54 | NO | | | NO | |
| 2150 | 2018 Jun. 18T20:15:35 | NO | | | NO | |
| 2150 | 2018 Jun. 19T21:58:37 | YES | MILD | | YES | 1 |
| 2150 | 2018 Jun. 20T19:43:33 | NO | | | NO | |
| 2150 | 2018 Jun. 21T21:20:46 | NO | | | NO | |
| 2150 | 2018 Jun. 23T18:15:54 | NO | | | NO | |
| 2150 | 2018 Jun. 24T20:27:44 | YES | MODERATE | | YES | 1 |
| 2150 | 2018 Jun. 25T19:16:11 | NO | | | NO | |
| 2150 | 2018 Jun. 26T18:33:21 | NO | | | NO | |
| 2150 | 2018 Jun. 27T21:21:08 | NO | | | NO | |
| 2150 | 2018 Jun. 28T21:18:41 | NO | | | NO | |
| 2150 | 2018 Jun. 29T22:00:38 | NO | | | NO | |
| 2150 | 2018 Jun. 30T21:24:24 | NO | | | NO | |
| 2150 | 2018 Jul. 1T19:47:08 | NO | | | NO | |
| 2150 | 2018 Jul. 2T19:41:57 | NO | | | NO | |
| 2150 | 2018 Jul. 3T22:00:26 | NO | | | NO | |
| 2150 | 2018 Jul. 4T20:34:29 | NO | | | NO | |
| 2150 | 2018 Jul. 5T22:01:06 | YES | MODERATE | | YES | 1 |
| 2150 | 2018 Jul. 6T22:00:51 | NO | | | NO | |
| 2150 | 2018 Jul. 7T22:00:27 | NO | | | NO | |
| 2150 | 2018 Jul. 8T22:00:27 | NO | | | NO | |
| 2150 | 2018 Jul. 9T22:03:08 | YES | MILD | | NO | |
| 2150 | 2018 Jul. 11T21:43:47 | NO | | | NO | |
| 2150 | 2018 Jul. 12T18:50:52 | YES | MILD | | YES | 1 |
| 2150 | 2018 Jul. 13T23:04:40 | NO | | | NO | |
| 2150 | 2018 Jul. 14T19:35:02 | NO | | | NO | |
| 2150 | 2018 Jul. 15T22:04:44 | YES | MILD | | NO | |
| 2150 | 2018 Jul. 17T18:32:48 | NO | | | NO | |
| 2150 | 2018 Jul. 21 | YES | MODERATE | | YES | 1 |
| 2150 | 2018 Jul. 22T18:22:18 | YES | MILD | | NO | |
| 2150 | 2018 Jul. 23T20:20:58 | YES | MILD | | YES | 1 |
| 2150 | 2018 Jul. 24T20:51:35 | YES | MILD | | NO | |
| 2150 | 2018 Jul. 25T20:11:18 | YES | MILD | | NO | |
| 2150 | 2018 Jul. 26T20:23:01 | NO | | | NO | |
| 2150 | 2018 Jul. 27T22:00:55 | YES | MODERATE | | YES | 1 |
| 2150 | 2018 Jul. 28T18:09:13 | YES | MODERATE | | YES | 1 |
| 2150 | 2018 Jul. 29T22:02:13 | YES | MILD | | NO | |
| 2150 | 2018 Jul. 30T19:45:05 | NO | | | NO | |
| 2150 | 2018 Jul. 31T22:02:37 | NO | | | NO | |
| 2150 | 2018 Aug. 1T20:26:55 | YES | MILD | | NO | |
| 2150 | 2018 Aug. 2T23:32:36 | YES | MODERATE | | YES | 1 |
| 2150 | 2018 Aug. 4T23:20:11 | NO | | | NO | |
| 2150 | 2018 Aug. 5T22:31:31 | YES | MILD | | YES | 1 |
| 2150 | 2018 Aug. 6T18:50:14 | NO | | | NO | |
| 2150 | 2018 Aug. 7T19:18:15 | NO | | | NO | |
| 2150 | 2018 Aug. 8T20:35:52 | NO | | | NO | |
| 2150 | 2018 Aug. 9T18:06:03 | YES | MILD | | NO | |
| 2150 | 2018 Aug. 10T22:06:30 | YES | MODERATE | | YES | 1 |
| 2150 | 2018 Aug. 11T18:15:11 | YES | MILD | | YES | 1 |
| 2150 | 2018 Aug. 12T18:03:07 | YES | MILD | | NO | |
| 2150 | 2018 Aug. 13T19:05:27 | YES | MODERATE | | YES | 1 |
| 2150 | 2018 Aug. 14T18:07:10 | YES | MODERATE | | YES | 1 |
| 2150 | 2018 Aug. 15T19:49:56 | YES | MODERATE | | YES | 1 |
| 2150 | 2018 Aug. 16T19:45:14 | YES | MILD | | YES | 1 |
| 2150 | 2018 Aug. 17T19:58:02 | NO | | | NO | |
| 2150 | 2018 Aug. 18T22:02:17 | NO | | | NO | |
| 2150 | 2018 Aug. 19T18:46:35 | NO | | | NO | |
| 2150 | 2018 Aug. 20T19:06:15 | YES | MODERATE | | YES | 1 |
| 2150 | 2018 Aug. 21T18:38:00 | NO | | | NO | |
| 2150 | 2018 Aug. 22T18:46:05 | NO | | | NO | |
| 2150 | 2018 Aug. 23T22:18:15 | NO | | | NO | |
| 2150 | 2018 Aug. 24T18:34:58 | NO | | | NO | |
| 2150 | 2018 Aug. 25T18:22:15 | NO | | | NO | |
| 2150 | 2018 Aug. 26T22:01:25 | YES | MODERATE | | YES | 1 |
| 2150 | 2018 Aug. 27T18:00:49 | YES | MODERATE | | YES | 1 |
| 2150 | 2018 Aug. 28T22:46:08 | NO | | | NO | |
| 2150 | 2018 Aug. 29T18:03:32 | NO | | | NO | |
| 2150 | 2018 Aug. 30T22:43:14 | NO | | | NO | |
| 2150 | 2018 Aug. 31T19:12:37 | YES | MODERATE | | YES | 1 |
| 2150 | 2018 Sep. 1T23:44:54 | NO | | | NO | |
| 2150 | 2018 Sep. 2T22:04:56 | YES | MODERATE | | YES | 1 |
| 2150 | 2018 Sep. 3T22:24:08 | NO | | | NO | |
| 2150 | 2018 Sep. 6T18:11:26 | NO | | | NO | |
| 2150 | 2018 Sep. 7T18:25:43 | NO | | | NO | |
| 2150 | 2018 Sep. 8T19:30:30 | YES | MODERATE | | YES | 1 |
| 2150 | 2018 Sep. 9T19:51:07 | NO | | | NO | |
| 2150 | 2018 Sep. 11T22:01:39 | NO | | | NO | |
| 2150 | 2018 Sep. 12T21:25:56 | NO | | | NO | |
| 2150 | 2018 Sep. 13T22:00:30 | NO | | | NO | |
| 2150 | 2018 Sep. 14T18:06:48 | NO | | | NO | |
| 2150 | 2018 Sep. 15T18:33:02 | NO | | | NO | |
| 2150 | 2018 Sep. 16T19:06:34 | NO | | | NO | |
| 2150 | 2018 Sep. 17T19:26:19 | NO | | | NO | |
| 2150 | 2018 Sep. 18T21:52:03 | YES | MILD | | YES | 1 |
| 2150 | 2018 Sep. 19T18:42:06 | NO | | | NO | |
| 2150 | 2018 Sep. 20T21:22:34 | NO | | | NO | |
| 2150 | 2018 Sep. 21T19:17:53 | NO | | | NO | |
| 2150 | 2018 Sep. 23T20:43:52 | NO | | | NO | |
| 2150 | 2018 Sep. 24T22:02:30 | YES | MODERATE | | YES | 1 |
| 2150 | 2018 Sep. 25T20:01:21 | YES | MODERATE | | YES | 1 |
| 2150 | 2018 Sep. 26T19:39:08 | YES | MODERATE | | YES | 1 |
| 2150 | 2018 Sep. 27T20:30:46 | NO | | | NO | |
| 2150 | 2018 Sep. 28T22:47:41 | YES | MODERATE | | YES | 1 |
| 2150 | 2018 Sep. 29 | YES | MODERATE | | YES | 1 |
| 2150 | 2018 Sep. 30T20:03:57 | YES | MODERATE | | YES | 1 |
| 2150 | 2018 Oct. 1T18:28:54 | NO | | | NO | |
| 2150 | 2018 Oct. 2T18:09:46 | NO | | | NO | |
| 2150 | 2018 Oct. 4T18:07:28 | YES | MILD | | YES | 1 |
| 2150 | 2018 Oct. 5T20:37:17 | NO | | | NO | |
| 2150 | 2018 Oct. 6 | YES | MODERATE | | YES | 1 |
| 2150 | 2018 Oct. 7T22:36:32 | YES | MILD | | YES | 1 |
| 2150 | 2018 Oct. 8 | YES | MODERATE | | NO | |
| 2150 | 2018 Oct. 9T20:19:20 | NO | | | NO | |
| 2150 | 2018 Oct. 10T22:04:31 | NO | | | NO | |
| 2150 | 2018 Oct. 11T22:00:48 | NO | | | NO | |
| 2150 | 2018 Oct. 13T18:08:31 | NO | | | NO | |
| 2150 | 2018 Oct. 15T18:10:00 | NO | | | NO | |
| 2150 | 2018 Oct. 16T19:30:45 | NO | | | NO | |
| 2150 | 2018 Oct. 17T18:17:34 | NO | | | NO | |
| 2150 | 2018 Oct. 18T18:05:36 | NO | | | NO | |
| 2150 | 2018 Oct. 19T18:15:59 | NO | | | NO | |
| 2150 | 2018 Oct. 20T18:13:47 | NO | | | NO | |
| 2150 | 2018 Oct. 21T20:05:45 | NO | | | NO | |
| 2150 | 2018 Oct. 22T22:00:22 | NO | | | NO | |
| 2150 | 2018 Oct. 23T22:00:19 | NO | | | NO | |
| 2150 | 2018 Oct. 24T18:34:46 | NO | | | NO | |
| 2150 | 2018 Oct. 25T22:01:21 | NO | | | NO | |
| 2150 | 2018 Oct. 26T19:48:16 | NO | | | NO | |
| 2150 | 2018 Oct. 27T18:00:51 | NO | | | NO | |
| 2150 | 2018 Oct. 28T18:02:09 | NO | | | NO | |
| 2150 | 2018 Oct. 30T22:18:38 | NO | | | NO | |
| 2150 | 2018 Oct. 31T18:21:57 | YES | MILD | | YES | 1 |
| 2150 | 2018 Nov. 2T22:01:26 | NO | | | NO | |
| 2150 | 2018 Nov. 3T18:01:01 | NO | | | NO | |
| 2150 | 2018 Nov. 4T20:00:29 | NO | | | NO | |
| 2150 | 2018 Nov. 5T22:47:33 | NO | | | NO | |
| 2150 | 2018 Nov. 6T21:46:51 | NO | | | NO | |
| 2150 | 2018 Nov. 7T21:15:48 | YES | MODERATE | | YES | 1 |
| 2150 | 2018 Nov. 8T18:22:36 | YES | MILD | | NO | |
| 2150 | 2018 Nov. 9T20:04:23 | YES | MILD | | NO | |
| 2150 | 2018 Nov. 10T18:01:53 | YES | MILD | | YES | 1 |
| 2150 | 2018 Nov. 11T21:17:02 | NO | | | NO | |
| 2150 | 2018 Nov. 12T18:18:31 | YES | MILD | | YES | 1 |
| 2150 | 2018 Nov. 13T18:10:27 | YES | MODERATE | | YES | 1 |
| 2150 | 2018 Nov. 14T22:00:27 | YES | MILD | | YES | 1 |
| 2150 | 2018 Nov. 15T19:19:23 | YES | MILD | | NO | |
| 2150 | 2018 Nov. 16T22:15:26 | YES | MILD | | NO | |
| 2150 | 2018 Nov. 17T18:18:34 | NO | | | NO | |
| 2150 | 2018 Nov. 19T21:19:20 | NO | | | NO | |
| 2150 | 2018 Nov. 20T22:00:42 | NO | | | NO | |
| 2150 | 2018 Nov. 21T18:01:37 | NO | | | NO | |
| 2150 | 2018 Nov. 22T18:54:33 | NO | | | NO | |
| 2150 | 2018 Nov. 23T22:04:28 | NO | | | NO | |
| 2150 | 2018 Nov. 24T19:28:21 | NO | | | NO | |
| 2150 | 2018 Nov. 25T22:01:27 | NO | | | NO | |
| 2150 | 2018 Nov. 26T22:01:30 | NO | | | NO | |
| 2150 | 2018 Nov. 27T21:54:26 | YES | MILD | | NO | |
| 2150 | 2018 Nov. 28T18:15:48 | NO | | | NO | |
| 2150 | 2018 Nov. 29T18:00:43 | NO | | | NO | |
| 2150 | 2018 Nov. 30T22:02:41 | NO | | | NO | |
| 2150 | 2018 Dec. 1T22:03:37 | YES | MODERATE | | YES | 1 |
| 2150 | 2018 Dec. 2T19:04:31 | YES | MILD | | NO | |
| 2150 | 2018 Dec. 3T21:01:03 | NO | | | NO | |
| 2150 | 2018 Dec. 4T19:15:41 | NO | | | NO | |
| 2150 | 2018 Dec. 5T20:46:48 | NO | | | NO | |
| 2150 | 2018 Dec. 6T18:02:56 | NO | | | NO | |
| 2150 | 2018 Dec. 7T19:00:03 | NO | | | NO | |
| 2150 | 2018 Dec. 8T19:04:42 | NO | | | NO | |
| 2150 | 2018 Dec. 9T18:05:17 | NO | | | NO | |
| 2150 | 2018 Dec. 10T18:22:47 | NO | | | NO | |
| 2150 | 2018 Dec. 11T19:37:41 | NO | | | NO | |
| 2150 | 2018 Dec. 12T20:36:15 | NO | | | NO | |
| 2150 | 2018 Dec. 13T18:21:25 | NO | | | NO | |
| 2150 | 2018 Dec. 14T18:01:37 | YES | MODERATE | | NO | |
| 2150 | 2018 Dec. 15T22:31:18 | NO | | | NO | |
| 2150 | 2018 Dec. 16T18:16:57 | NO | | | NO | |
| 2150 | 2018 Dec. 17T18:52:11 | NO | | | NO | |
| 2150 | 2018 Dec. 18T18:23:12 | NO | | | NO | |
| 2150 | 2018 Dec. 19T18:17:14 | NO | | | NO | |
| 2150 | 2018 Dec. 20T18:53:42 | NO | | | NO | |
| 2150 | 2018 Dec. 21T18:34:03 | NO | | | NO | |
| 2150 | 2018 Dec. 22T19:35:22 | NO | | | NO | |
| 2150 | 2018 Dec. 23T19:00:26 | YES | MODERATE | | YES | 1 |
| 2150 | 2018 Dec. 24T22:14:20 | YES | MILD | | NO | |
| 2150 | 2018 Dec. 25T23:43:47 | NO | | | NO | |
| 2150 | 2018 Dec. 26T18:07:50 | YES | MILD | | YES | 1 |
| 2150 | 2018 Dec. 27T18:04:46 | NO | | | NO | |
| 2150 | 2018 Dec. 28T19:24:58 | YES | MILD | | YES | 1 |
| 2150 | 2019 Jan. 1T19:48:21 | NO | | | NO | |
| 2150 | 2019 Jan. 2T18:36:43 | NO | | | NO | |
| 2150 | 2019 Jan. 3T18:22:45 | NO | | | NO | |
| 2150 | 2019 Jan. 4T20:57:53 | NO | | | NO | |
| 2150 | 2019 Jan. 5T18:18:45 | NO | | | NO | |
| 2150 | 2019 Jan. 6T18:05:06 | NO | | | NO | |
| 2150 | 2019 Jan. 7T20:03:16 | NO | | | NO | |
| 2150 | 2019 Jan. 8T19:23:00 | NO | | | NO | |
| 2150 | 2019 Jan. 9T19:19:51 | NO | | | NO | |
| 2150 | 2019 Jan. 10T19:07:18 | NO | | | NO | |
| 2150 | 2019 Jan. 11T18:49:03 | NO | | | NO | |
| 2150 | 2019 Jan. 12T18:31:12 | NO | | | NO | |
| 2150 | 2019 Jan. 13T18:38:21 | YES | MILD | | YES | 1 |
| 2150 | 2019 Jan. 14T22:00:23 | NO | | | NO | |
| 2150 | 2019 Jan. 15T18:01:10 | YES | MILD | | YES | 1 |
| 2150 | 2019 Jan. 16T18:39:48 | NO | | | NO | |
| 2150 | 2019 Jan. 17T22:00:25 | NO | | | NO | |
| 2150 | 2019 Jan. 18T18:14:47 | NO | | | NO | |
| 2150 | 2019 Jan. 20T23:30:22 | NO | | | NO | |
| 2150 | 2019 Jan. 21T21:08:25 | NO | | | NO | |
| 2150 | 2019 Jan. 22T21:46:25 | NO | | | NO | |
| 2150 | 2019 Jan. 23T20:50:44 | NO | | | NO | |
| 2150 | 2019 Jan. 25T18:51:30 | NO | | | NO | |
| 2150 | 2019 Jan. 26T18:42:58 | NO | | | NO | |
| 2150 | 2019 Jan. 27T18:20:14 | YES | MODERATE | | YES | 1 |
| 2150 | 2019 Jan. 28T18:11:13 | NO | | | NO | |
| 2150 | 2019 Jan. 30T21:22:11 | NO | | | NO | |
| 2150 | 2019 Jan. 31T18:20:24 | NO | | | NO | |
| 2150 | 2019 Feb. 1T20:17:25 | YES | MODERATE | | YES | 1 |
| 2150 | 2019 Feb. 2T18:02:10 | YES | MODERATE | | YES | 1 |
| 2150 | 2019 Feb. 3T18:57:27 | NO | | | NO | |
| 2150 | 2019 Feb. 4T19:00:29 | NO | | | NO | |
| 2150 | 2019 Feb. 5T18:56:53 | YES | MILD | | NO | |
| 2150 | 2019 Feb. 6T19:03:59 | NO | | | NO | |
| 2150 | 2019 Feb. 7T18:54:18 | NO | | | NO | |
| 2150 | 2019 Feb. 8T22:00:25 | NO | | | NO | |
| 2150 | 2019 Feb. 9T18:33:36 | NO | | | NO | |
| 2150 | 2019 Feb. 10T22:00:45 | YES | MILD | | NO | |
| 2150 | 2019 Feb. 11T19:43:09 | YES | MODERATE | | YES | 1 |
| 2150 | 2019 Feb. 12T22:02:22 | YES | MILD | | NO | |
| 2150 | 2019 Feb. 13T21:45:00 | NO | | | NO | |
| 2150 | 2019 Feb. 14T18:50:13 | NO | | | NO | |
| 2150 | 2019 Feb. 17T18:58:44 | YES | MODERATE | | YES | 1 |
| 2150 | 2019 Feb. 20T19:01:47 | YES | MODERATE | | YES | 1 |
| 2150 | 2019 Feb. 21T18:02:00 | YES | MODERATE | | YES | 1 |
| 2150 | 2019 Feb. 22T19:50:16 | YES | MILD | | NO | |
| 2150 | 2019 Feb. 23T19:30:41 | NO | | | NO | |
| 2150 | 2019 Feb. 24T21:10:18 | NO | | | NO | |
| 2150 | 2019 Feb. 25T18:42:59 | NO | | | NO | |
| 2150 | 2019 Feb. 26T18:17:35 | NO | | | NO | |
| 2150 | 2019 Feb. 27T20:41:26 | NO | | | NO | |
| 2150 | 2019 Feb. 28T18:10:23 | YES | MODERATE | | YES | 1 |
| 2150 | 2019 Mar. 1T19:11:41 | YES | MODERATE | | NO | |
| 2150 | 2019 Mar. 2T18:40:18 | NO | | | NO | |
| 2150 | 2019 Mar. 3T18:26:25 | NO | | | NO | |
| 2150 | 2019 Mar. 4T18:11:24 | NO | | | NO | |
| 2150 | 2019 Mar. 5T18:27:26 | NO | | | NO | |
| 2150 | 2019 Mar. 6T18:21:04 | NO | | | NO | |
| 2150 | 2019 Mar. 7T18:05:48 | NO | | | NO | |
| 2150 | 2019 Mar. 8T18:25:09 | NO | | | NO | |
| 2150 | 2019 Mar. 10T23:00:36 | NO | | | NO | |
| 2150 | 2019 Mar. 11T18:06:29 | NO | | | NO | |
| 2150 | 2019 Mar. 12T20:26:56 | NO | | | NO | |
| 2150 | 2019 Mar. 13T20:33:16 | NO | | | NO | |
| 2150 | 2019 Mar. 14T18:00:44 | YES | MILD | | NO | |
| 2150 | 2019 Mar. 15T20:03:12 | YES | MILD | | YES | 1 |
| 2150 | 2019 Mar. 16T18:04:46 | NO | | | NO | |
| 2150 | 2019 Mar. 17T23:29:31 | NO | | | NO | |
| 2150 | 2019 Mar. 18T23:22:22 | NO | | | NO | |
| 2150 | 2019 Mar. 19T18:51:30 | NO | | | NO | |
| 2150 | 2019 Mar. 20T18:58:50 | NO | | | NO | |
| 2150 | 2019 Mar. 22T19:17:59 | YES | MILD | | YES | 1 |
| 2150 | 2019 Mar. 24T22:11:06 | YES | MILD | | YES | 1 |
| 2150 | 2019 Mar. 25T18:37:43 | NO | | | NO | |
| 2150 | 2019 Mar. 26T19:26:48 | NO | | | NO | |
| 2150 | 2019 Mar. 27T18:13:19 | YES | MILD | | NO | |
| 2150 | 2019 Mar. 28T19:32:12 | NO | | | NO | |
| 2150 | 2019 Mar. 29T19:30:59 | NO | | | NO | |
| 2150 | 2019 Mar. 30T18:31:29 | NO | | | NO | |
| 2150 | 2019 Mar. 31T18:18:58 | NO | | | NO | |
| 2150 | 2019 Apr. 1T18:51:43 | YES | SEVERE | | YES | 1 |
| 2150 | 2019 Apr. 2T21:15:44 | NO | | | NO | |
| 2150 | 2019 Apr. 3T22:01:24 | YES | MODERATE | | YES | 1 |
| 2150 | 2019 Apr. 4T19:37:10 | YES | MILD | | NO | |
| 2150 | 2019 Apr. 5T19:09:52 | YES | MILD | | NO | |
| 2150 | 2019 Apr. 6T18:02:02 | YES | MILD | | NO | |
| 2150 | 2019 Apr. 7T18:40:33 | NO | | | NO | |
| 2150 | 2019 Apr. 8T18:41:50 | NO | | | NO | |
| 2150 | 2019 Apr. 9T18:06:21 | NO | | | NO | |
| 2150 | 2019 Apr. 10T22:01:28 | NO | | | NO | |
| 2150 | 2019 Apr. 11T18:21:46 | NO | | | NO | |
| 2150 | 2019 Apr. 12T18:00:55 | YES | MILD | | NO | |
| 2150 | 2019 Apr. 14T22:31:51 | NO | | | NO | |
| 2150 | 2019 Apr. 15T19:15:30 | YES | MILD | | NO | |
| 2150 | 2019 Apr. 16T19:17:44 | NO | | | NO | |
| 2150 | 2019 Apr. 17T23:23:15 | NO | | | NO | |
| 2150 | 2019 Apr. 18T22:04:46 | NO | | | NO | |
| 2150 | 2019 Apr. 19T21:46:34 | NO | | | NO | |
| 2150 | 2019 Apr. 20T18:43:06 | YES | MILD | | NO | |
| 2150 | 2019 Apr. 21T18:05:58 | NO | | | NO | |
| 2150 | 2019 Apr. 22T18:36:01 | NO | | | NO | |
| 2150 | 2019 Apr. 23T19:33:48 | NO | | | NO | |
| 2150 | 2019 Apr. 24T22:13:58 | YES | MODERATE | | NO | |
| 2150 | 2019 Apr. 25T19:11:45 | NO | | | NO | |
| 2150 | 2019 Apr. 26T20:04:40 | NO | | | NO | |
| 2150 | 2019 Apr. 28T21:56:29 | NO | | | NO | |
| 2150 | 2019 Apr. 29T20:12:04 | NO | | | NO | |
| 2150 | 2019 Apr. 30T21:56:49 | YES | MILD | | NO | |
| 2150 | 2019 May 1T18:28:20 | YES | MILD | | NO | |
| 2150 | 2019 May 4T20:35:13 | NO | | | NO | |
| 2150 | 2019 May 5T21:17:16 | NO | | | NO | |
| 2150 | 2019 May 6T20:23:45 | NO | | | NO | |
| 2150 | 2019 May 7T22:01:48 | NO | | | NO | |
| 2150 | 2019 May 8T20:57:26 | NO | | | NO | |
| 2150 | 2019 May 9T22:01:25 | NO | | | NO | |
| 2150 | 2019 May 10T18:18:57 | NO | | | NO | |
| 2150 | 2019 May 12T18:38:59 | NO | | | NO | |
| 2150 | 2019 May 13T11:19:39 | NO | | | NO | |

Subject 2150 is a 29 year-old Caucasian female with history of 9-14 migraine attacks (with aura) per month since the age of 24. Past medication used to treat her headache has been rizatriptan, ibuprofen, ubidecarenone, and fioricet. Additionally, she has been receiving Botox® injections on 7 Jun. 2018, 7 Sep. 2018, 5 Dec. 2018, and 14 Mar. 2019.

TABLE 20

| | | | | Take other | Take study | |
|---|---|---|---|---|---|---|
| | | Did you have | | med | medication | |
| | | a migraine | | [Observation | [Treatment | Number of |
| Subject | Date/Time of Finding | headache? | Severity | phase] | phase] | tablets |
| 1711 | 2018 Feb. 20T18:04:00 | YES | MILD | YES | | |
| 1711 | 2018 Feb. 21T19:12:25 | NO | | | | |
| 1711 | 2018 Feb. 22T18:16:32 | YES | MILD | YES | | |
| 1711 | 2018 Feb. 24T18:05:21 | NO | | | | |
| 1711 | 2018 Feb. 25T18:30:24 | NO | | | | |
| 1711 | 2018 Feb. 26T18:03:23 | NO | | | | |
| 1711 | 2018 Feb. 27T18:47:27 | NO | | | | |
| 1711 | 2018 Feb. 28T18:00:13 | YES | MODERATE | NO | | |
| 1711 | 2018 Mar. 1T18:01:24 | YES | MODERATE | YES | | |
| 1711 | 2018 Mar. 2T18:05:54 | NO | | | | |
| 1711 | 2018 Mar. 3T18:45:20 | YES | MILD | YES | | |
| 1711 | 2018 Mar. 4T18:14:17 | NO | | | | |
| 1711 | 2018 Mar. 5T19:34:11 | NO | | | | |
| 1711 | 2018 Mar. 6T18:17:47 | YES | MODERATE | YES | | |
| 1711 | 2018 Mar. 7T18:30:27 | NO | | | | |
| 1711 | 2018 Mar. 8T19:23:24 | NO | | | | |
| 1711 | 2018 Mar. 12T19:47:51 | YES | MODERATE | YES | | |
| 1711 | 2018 Mar. 13T18:45:38 | YES | MILD | YES | | |
| 1711 | 2018 Mar. 14T18:38:06 | NO | | | | |
| 1711 | 2018 Mar. 15T18:00:23 | NO | | | | |
| 1711 | 2018 Mar. 16T21:33:18 | YES | MILD | YES | | |
| 1711 | 2018 Mar. 17T18:58:35 | YES | MODERATE | YES | | |
| 1711 | 2018 Mar. 18T18:02:29 | NO | | | | |
| 1711 | 2018 Mar. 19T19:47:35 | NO | | | | |
| 1711 | 2018 Mar. 20T18:15:30 | NO | | | | |
| 1711 | 2018 Mar. 21T18:02:17 | NO | | | | |
| 1711 | 2018 Mar. 22T18:01:31 | NO | | | NO | |
| 1711 | 2018 Mar. 23T18:45:16 | NO | | | NO | |
| 1711 | 2018 Mar. 24T18:07:30 | NO | | | NO | |
| 1711 | 2018 Mar. 25T18:50:31 | NO | | | NO | |
| 1711 | 2018 Mar. 26T21:10:43 | YES | MODERATE | | YES | 1 |
| 1711 | 2018 Mar. 27T18:49:23 | YES | MODERATE | | YES | 1 |
| 1711 | 2018 Mar. 28T18:08:41 | YES | SEVERE | | YES | 1 |
| 1711 | 2018 Mar. 29T18:13:26 | NO | | | NO | |
| 1711 | 2018 Mar. 31T18:04:47 | NO | | | NO | |
| 1711 | 2018 Apr. 1T18:16:39 | NO | | | NO | |
| 1711 | 2018 Apr. 2T20:28:00 | NO | | | NO | |
| 1711 | 2018 Apr. 3T18:31:07 | NO | | | NO | |
| 1711 | 2018 Apr. 4T18:59:20 | NO | | | NO | |
| 1711 | 2018 Apr. 5T18:15:27 | YES | MODERATE | | YES | 1 |
| 1711 | 2018 Apr. 6T18:00:56 | NO | | | NO | |
| 1711 | 2018 Apr. 7T18:15:37 | NO | | | NO | |
| 1711 | 2018 Apr. 8T18:30:31 | NO | | | NO | |
| 1711 | 2018 Apr. 10T18:00:25 | NO | | | NO | |
| 1711 | 2018 Apr. 11T18:46:26 | NO | | | NO | |
| 1711 | 2018 Apr. 12T18:38:23 | NO | | | NO | |
| 1711 | 2018 Apr. 15T18:02:08 | NO | | | NO | |
| 1711 | 2018 Apr. 16T18:00:13 | NO | | | NO | |
| 1711 | 2018 Apr. 17T18:01:05 | NO | | | NO | |
| 1711 | 2018 Apr. 18T18:01:42 | YES | MODERATE | | YES | 1 |
| 1711 | 2018 Apr. 19T18:04:15 | YES | MODERATE | | YES | 1 |
| 1711 | 2018 Apr. 20T18:00:24 | NO | | | NO | |
| 1711 | 2018 Apr. 21T20:29:43 | NO | | | NO | |
| 1711 | 2018 Apr. 22T18:35:43 | NO | | | NO | |
| 1711 | 2018 Apr. 23T18:18:40 | NO | | | NO | |
| 1711 | 2018 Apr. 25T21:07:28 | YES | SEVERE | | YES | 1 |
| 1711 | 2018 Apr. 26T18:20:32 | YES | MILD | | YES | 1 |
| 1711 | 2018 Apr. 28T18:47:12 | NO | | | NO | |
| 1711 | 2018 Apr. 29T18:00:26 | NO | | | NO | |
| 1711 | 2018 Apr. 30T23:00:24 | NO | | | NO | |
| 1711 | 2018 May 1T18:45:33 | YES | MODERATE | | YES | 1 |
| 1711 | 2018 May 3T18:05:41 | NO | | | NO | |
| 1711 | 2018 May 4T21:04:33 | NO | | | NO | |
| 1711 | 2018 May 5T23:15:53 | YES | MODERATE | | YES | 1 |
| 1711 | 2018 May 6T18:45:50 | NO | | | NO | |
| 1711 | 2018 May 7T23:06:59 | YES | MILD | | YES | 1 |
| 1711 | 2018 May 8T23:09:31 | NO | | | NO | |
| 1711 | 2018 May 9T22:34:30 | NO | | | NO | |
| 1711 | 2018 May 10T18:31:07 | NO | | | NO | |
| 1711 | 2018 May 11T19:16:51 | NO | | | NO | |
| 1711 | 2018 May 12T18:46:55 | NO | | | NO | |
| 1711 | 2018 May 13T18:00:44 | NO | | | NO | |
| 1711 | 2018 May 14T22:22:54 | NO | | | NO | |
| 1711 | 2018 May 15T18:22:24 | NO | | | NO | |
| 1711 | 2018 May 16T18:20:36 | NO | | | NO | |
| 1711 | 2018 May 17T18:02:03 | NO | | | NO | |
| 1711 | 2018 May 18T22:35:38 | NO | | | NO | |
| 1711 | 2018 May 20T18:05:21 | NO | | | NO | |
| 1711 | 2018 May 21T20:27:03 | NO | | | NO | |
| 1711 | 2018 May 23T21:32:52 | YES | MILD | | YES | 1 |
| 1711 | 2018 May 24T21:11:26 | NO | | | NO | |
| 1711 | 2018 May 25T20:14:14 | YES | MODERATE | | YES | 1 |
| 1711 | 2018 May 26T18:04:34 | NO | | | NO | |
| 1711 | 2018 May 27T19:00:36 | NO | | | NO | |
| 1711 | 2018 May 28T23:05:02 | NO | | | NO | |
| 1711 | 2018 May 29T19:46:26 | NO | | | NO | |
| 1711 | 2018 May 30T19:48:41 | NO | | | NO | |
| 1711 | 2018 Jun. 1T19:15:30 | NO | | | NO | |
| 1711 | 2018 Jun. 2T19:01:01 | NO | | | NO | |
| 1711 | 2018 Jun. 3T18:50:16 | NO | | | NO | |
| 1711 | 2018 Jun. 5T18:43:01 | YES | MODERATE | | YES | 1 |
| 1711 | 2018 Jun. 6T19:19:38 | NO | | | NO | |
| 1711 | 2018 Jun. 10T23:20:59 | NO | | | NO | |
| 1711 | 2018 Jun. 11T19:50:33 | NO | | | NO | |
| 1711 | 2018 Jun. 12T19:09:45 | NO | | | NO | |
| 1711 | 2018 Jun. 13T23:21:52 | NO | | | NO | |
| 1711 | 2018 Jun. 14T19:34:10 | NO | | | NO | |
| 1711 | 2018 Jun. 15T23:45:27 | NO | | | NO | |
| 1711 | 2018 Jun. 16T22:50:09 | YES | MODERATE | | YES | 1 |
| 1711 | 2018 Jun. 17T18:27:00 | NO | | | NO | |
| 1711 | 2018 Jun. 18T23:24:33 | NO | | | NO | |
| 1711 | 2018 Jun. 20T19:31:06 | NO | | | NO | |
| 1711 | 2018 Jun. 22T22:30:46 | NO | | | NO | |
| 1711 | 2018 Jun. 23T21:55:50 | NO | | | NO | |
| 1711 | 2018 Jun. 24T18:08:21 | NO | | | NO | |
| 1711 | 2018 Jun. 25T19:18:26 | NO | | | NO | |
| 1711 | 2018 Jun. 26T20:57:49 | YES | MODERATE | | YES | 1 |
| 1711 | 2018 Jun. 27T23:41:52 | NO | | | NO | |
| 1711 | 2018 Jun. 29T19:00:20 | NO | | | NO | |
| 1711 | 2018 Jun. 30T23:56:52 | NO | | | NO | |
| 1711 | 2018 Jul. 2T22:54:45 | YES | MODERATE | | YES | 1 |
| 1711 | 2018 Jul. 3T19:25:43 | NO | | | NO | |
| 1711 | 2018 Jul. 4T18:42:48 | NO | | | NO | |
| 1711 | 2018 Jul. 5T21:49:50 | NO | | | NO | |
| 1711 | 2018 Jul. 6T22:17:56 | NO | | | NO | |
| 1711 | 2018 Jul. 7T19:47:26 | NO | | | NO | |
| 1711 | 2018 Jul. 8T23:20:16 | YES | MODERATE | | YES | 1 |
| 1711 | 2018 Jul. 9T20:54:26 | NO | | | NO | |
| 1711 | 2018 Jul. 10T22:56:12 | NO | | | NO | |
| 1711 | 2018 Jul. 11T19:31:49 | YES | MODERATE | | YES | 1 |
| 1711 | 2018 Jul. 12T21:28:47 | NO | | | NO | |
| 1711 | 2018 Jul. 14T23:18:05 | NO | | | NO | |
| 1711 | 2018 Jul. 16T18:46:20 | NO | | | NO | |
| 1711 | 2018 Jul. 18T21:28:00 | NO | | | NO | |
| 1711 | 2018 Jul. 19T22:59:18 | YES | MILD | | YES | 1 |
| 1711 | 2018 Jul. 20T23:20:26 | NO | | | NO | |
| 1711 | 2018 Jul. 22T22:35:17 | NO | | | NO | |
| 1711 | 2018 Jul. 23T23:36:59 | YES | MODERATE | | YES | 1 |
| 1711 | 2018 Jul. 24T23:29:54 | NO | | | NO | |
| 1711 | 2018 Jul. 25T23:53:47 | NO | | | NO | |
| 1711 | 2018 Jul. 26T23:52:37 | NO | | | NO | |
| 1711 | 2018 Jul. 27T23:08:41 | NO | | | NO | |
| 1711 | 2018 Jul. 28T23:05:16 | NO | | | NO | |
| 1711 | 2018 Jul. 29T19:34:28 | NO | | | NO | |
| 1711 | 2018 Jul. 30T19:04:28 | NO | | | NO | |
| 1711 | 2018 Aug. 1T19:15:27 | NO | | | NO | |
| 1711 | 2018 Aug. 2T19:03:26 | NO | | | NO | |
| 1711 | 2018 Aug. 3T19:19:33 | NO | | | NO | |
| 1711 | 2018 Aug. 4T19:01:36 | NO | | | NO | |
| 1711 | 2018 Aug. 5T23:33:43 | NO | | | NO | |
| 1711 | 2018 Aug. 6T19:18:00 | YES | MODERATE | | YES | 1 |
| 1711 | 2018 Aug. 7T23:44:55 | NO | | | NO | |
| 1711 | 2018 Aug. 8T19:05:47 | YES | MILD | | YES | 1 |
| 1711 | 2018 Aug. 9T23:03:21 | NO | | | NO | |
| 1711 | 2018 Aug. 10T23:50:02 | NO | | | NO | |
| 1711 | 2018 Aug. 11T19:48:36 | YES | MILD | | YES | 1 |
| 1711 | 2018 Aug. 13T23:11:15 | NO | | | NO | |
| 1711 | 2018 Aug. 14T23:05:06 | NO | | | NO | |
| 1711 | 2018 Aug. 15T19:19:09 | YES | MILD | | YES | 1 |
| 1711 | 2018 Aug. 16T19:21:25 | NO | | | NO | |
| 1711 | 2018 Aug. 17T19:04:41 | NO | | | NO | |
| 1711 | 2018 Aug. 18T19:03:29 | NO | | | NO | |
| 1711 | 2018 Aug. 19T18:10:35 | NO | | | NO | |
| 1711 | 2018 Aug. 20T19:02:21 | NO | | | NO | |
| 1711 | 2018 Aug. 22T22:29:56 | NO | | | NO | |
| 1711 | 2018 Aug. 23T19:26:46 | NO | | | NO | |
| 1711 | 2018 Aug. 25T19:00:48 | NO | | | NO | |
| 1711 | 2018 Aug. 26T19:00:35 | NO | | | NO | |
| 1711 | 2018 Aug. 27T18:20:18 | NO | | | NO | |
| 1711 | 2018 Aug. 28T19:04:20 | YES | MODERATE | | YES | 1 |
| 1711 | 2018 Aug. 29T22:03:57 | NO | | | NO | |
| 1711 | 2018 Aug. 30T19:16:29 | NO | | | NO | |
| 1711 | 2018 Aug. 31T19:30:27 | NO | | | NO | |
| 1711 | 2018 Sep. 1T22:51:25 | NO | | | NO | |
| 1711 | 2018 Sep. 2T21:30:44 | NO | | | NO | |
| 1711 | 2018 Sep. 3T19:00:23 | NO | | | NO | |
| 1711 | 2018 Sep. 4T19:03:32 | NO | | | NO | |
| 1711 | 2018 Sep. 5T19:00:24 | NO | | | NO | |
| 1711 | 2018 Sep. 6T19:01:40 | NO | | | NO | |
| 1711 | 2018 Sep. 7T19:02:38 | NO | | | NO | |
| 1711 | 2018 Sep. 9T19:01:58 | NO | | | NO | |
| 1711 | 2018 Sep. 10T19:02:28 | NO | | | NO | |
| 1711 | 2018 Sep. 11T19:00:47 | YES | MODERATE | | YES | 1 |
| 1711 | 2018 Sep. 13T18:26:09 | NO | | | NO | |
| 1711 | 2018 Sep. 14T19:00:22 | NO | | | NO | |
| 1711 | 2018 Sep. 15T19:15:28 | NO | | | NO | |
| 1711 | 2018 Sep. 16T19:15:23 | NO | | | NO | |
| 1711 | 2018 Sep. 17T19:00:57 | NO | | | NO | |
| 1711 | 2018 Sep. 18T19:00:25 | NO | | | NO | |
| 1711 | 2018 Sep. 19T19:00:31 | NO | | | NO | |
| 1711 | 2018 Sep. 21T19:50:27 | NO | | | NO | |
| 1711 | 2018 Sep. 23T19:00:25 | NO | | | NO | |
| 1711 | 2018 Sep. 24T19:00:40 | NO | | | NO | |
| 1711 | 2018 Sep. 25T19:00:39 | NO | | | NO | |
| 1711 | 2018 Sep. 26T19:00:34 | NO | | | NO | |
| 1711 | 2018 Sep. 27T19:32:21 | NO | | | NO | |
| 1711 | 2018 Sep. 28T19:00:34 | NO | | | NO | |
| 1711 | 2018 Sep. 29T19:00:24 | NO | | | NO | |
| 1711 | 2018 Sep. 30T21:06:04 | YES | MILD | | YES | 1 |
| 1711 | 2018 Oct. 1T21:50:12 | NO | | | NO | |
| 1711 | 2018 Oct. 2T20:50:11 | YES | MODERATE | | YES | 1 |
| 1711 | 2018 Oct. 4T19:01:56 | NO | | | NO | |
| 1711 | 2018 Oct. 9T22:54:45 | NO | | | NO | |
| 1711 | 2018 Oct. 11T23:24:31 | NO | | | NO | |
| 1711 | 2018 Oct. 12T21:01:31 | NO | | | NO | |
| 1711 | 2018 Oct. 13T19:02:05 | NO | | | NO | |
| 1711 | 2018 Oct. 14T21:29:35 | NO | | | NO | |
| 1711 | 2018 Oct. 17T19:01:24 | YES | SEVERE | | YES | 1 |
| 1711 | 2018 Oct. 18T23:08:01 | NO | | | NO | |
| 1711 | 2018 Oct. 19T19:03:37 | NO | | | NO | |
| 1711 | 2018 Oct. 21T19:16:22 | NO | | | NO | |
| 1711 | 2018 Oct. 22T22:01:49 | NO | | | NO | |
| 1711 | 2018 Oct. 23T19:15:22 | NO | | | NO | |
| 1711 | 2018 Oct. 25T19:00:42 | YES | SEVERE | | YES | 1 |
| 1711 | 2018 Oct. 26T19:18:19 | NO | | | NO | |
| 1711 | 2018 Oct. 27T19:47:37 | NO | | | NO | |
| 1711 | 2018 Oct. 30T18:19:07 | NO | | | NO | |
| 1711 | 2018 Oct. 31T19:00:34 | NO | | | NO | |
| 1711 | 2018 Nov. 1T19:48:30 | NO | | | NO | |
| 1711 | 2018 Nov. 9T21:15:15 | NO | | | NO | |
| 1711 | 2018 Nov. 10T20:05:32 | YES | MILD | | YES | 1 |
| 1711 | 2018 Nov. 11T21:08:33 | NO | | | NO | |
| 1711 | 2018 Nov. 12T20:58:41 | YES | MILD | | YES | 1 |
| 1711 | 2018 Nov. 13T23:02:36 | NO | | | NO | |
| 1711 | 2018 Nov. 14T19:00:21 | YES | MILD | | YES | 1 |
| 1711 | 2018 Nov. 15T19:45:35 | NO | | | NO | |
| 1711 | 2018 Nov. 16T19:35:31 | NO | | | NO | |
| 1711 | 2018 Nov. 17T19:02:21 | NO | | | NO | |
| 1711 | 2018 Nov. 18T22:01:49 | YES | SEVERE | | YES | 1 |
| 1711 | 2018 Nov. 19T19:01:37 | NO | | | NO | |
| 1711 | 2018 Nov. 20T19:21:29 | NO | | | NO | |
| 1711 | 2018 Nov. 21T22:57:01 | NO | | | NO | |
| 1711 | 2018 Nov. 22T23:40:35 | NO | | | NO | |
| 1711 | 2018 Nov. 24T23:06:52 | NO | | | NO | |
| 1711 | 2018 Nov. 26T22:44:31 | NO | | | NO | |
| 1711 | 2018 Nov. 27T23:32:19 | YES | SEVERE | | YES | 1 |
| 1711 | 2018 Nov. 28T19:00:21 | NO | | | NO | |
| 1711 | 2018 Nov. 29T19:00:26 | NO | | | NO | |
| 1711 | 2018 Nov. 30T23:47:21 | NO | | | NO | |
| 1711 | 2018 Dec. 1T18:45:23 | NO | | | NO | |
| 1711 | 2018 Dec. 4T23:15:20 | YES | MODERATE | | YES | 1 |
| 1711 | 2018 Dec. 6T18:40:09 | NO | | | NO | |
| 1711 | 2018 Dec. 7T23:13:07 | NO | | | NO | |
| 1711 | 2018 Dec. 9T23:44:27 | NO | | | NO | |
| 1711 | 2018 Dec. 10T23:49:14 | NO | | | NO | |
| 1711 | 2018 Dec. 11T23:16:40 | NO | | | NO | |
| 1711 | 2018 Dec. 13T19:04:31 | NO | | | NO | |
| 1711 | 2018 Dec. 14T18:41:12 | YES | MODERATE | | YES | 1 |
| 1711 | 2018 Dec. 15T19:46:58 | NO | | | NO | |
| 1711 | 2018 Dec. 16T19:46:20 | NO | | | NO | |
| 1711 | 2018 Dec. 17T22:40:36 | NO | | | NO | |
| 1711 | 2018 Dec. 18T22:43:36 | NO | | | NO | |
| 1711 | 2018 Dec. 19T21:49:01 | NO | | | NO | |
| 1711 | 2018 Dec. 20T19:35:31 | NO | | | NO | |
| 1711 | 2018 Dec. 21T19:45:54 | YES | SEVERE | | YES | 1 |
| 1711 | 2018 Dec. 22T19:19:50 | YES | MILD | | YES | 1 |
| 1711 | 2018 Dec. 23T19:00:37 | NO | | | NO | |
| 1711 | 2018 Dec. 24T23:16:55 | NO | | | NO | |
| 1711 | 2018 Dec. 25T18:00:59 | NO | | | NO | |
| 1711 | 2018 Dec. 26T18:30:44 | YES | MILD | | YES | 1 |
| 1711 | 2018 Dec. 27T18:40:34 | NO | | | NO | |
| 1711 | 2018 Dec. 28T22:14:20 | NO | | | NO | |
| 1711 | 2018 Dec. 29T19:01:09 | NO | | | NO | |
| 1711 | 2018 Dec. 30T19:01:30 | NO | | | NO | |
| 1711 | 2018 Dec. 31T19:00:15 | NO | | | NO | |
| 1711 | 2019 Jan. 1T19:05:22 | YES | SEVERE | | YES | 1 |
| 1711 | 2019 Jan. 2T19:31:55 | NO | | | NO | |
| 1711 | 2019 Jan. 4T22:05:20 | NO | | | NO | |
| 1711 | 2019 Jan. 5T19:01:21 | NO | | | NO | |
| 1711 | 2019 Jan. 6T19:06:51 | NO | | | NO | |
| 1711 | 2019 Jan. 7T20:45:28 | NO | | | NO | |
| 1711 | 2019 Jan. 8T19:38:46 | NO | | | NO | |
| 1711 | 2019 Jan. 9T18:27:01 | NO | | | NO | |
| 1711 | 2019 Jan. 10T23:00:09 | YES | MILD | | YES | 1 |
| 1711 | 2019 Jan. 11T19:03:39 | NO | | | NO | |
| 1711 | 2019 Jan. 12T21:40:32 | NO | | | NO | |
| 1711 | 2019 Jan. 13T19:00:48 | NO | | | NO | |
| 1711 | 2019 Jan. 14T19:00:33 | NO | | | NO | |
| 1711 | 2019 Jan. 15T23:15:21 | NO | | | NO | |
| 1711 | 2019 Jan. 16T19:20:16 | NO | | | NO | |
| 1711 | 2019 Jan. 17T23:17:09 | NO | | | NO | |
| 1711 | 2019 Jan. 18T23:15:27 | NO | | | NO | |
| 1711 | 2019 Jan. 19T19:18:47 | NO | | | NO | |
| 1711 | 2019 Jan. 20T19:00:28 | NO | | | NO | |
| 1711 | 2019 Jan. 21T19:01:33 | NO | | | NO | |
| 1711 | 2019 Jan. 22T23:15:19 | NO | | | NO | |
| 1711 | 2019 Jan. 23T19:30:21 | NO | | | NO | |
| 1711 | 2019 Jan. 24T19:08:27 | NO | | | NO | |
| 1711 | 2019 Jan. 25T19:45:43 | NO | | | NO | |
| 1711 | 2019 Jan. 26T19:02:33 | NO | | | NO | |
| 1711 | 2019 Jan. 27T19:00:50 | NO | | | NO | |
| 1711 | 2019 Jan. 28T23:11:57 | YES | MODERATE | | YES | 1 |
| 1711 | 2019 Jan. 29T23:15:21 | NO | | | NO | |
| 1711 | 2019 Jan. 30T19:00:31 | NO | | | NO | |
| 1711 | 2019 Jan. 31T21:59:04 | NO | | | NO | |
| 1711 | 2019 Feb. 1T18:54:37 | NO | | | NO | |
| 1711 | 2019 Feb. 2T19:00:26 | NO | | | NO | |
| 1711 | 2019 Feb. 3T19:19:06 | NO | | | NO | |
| 1711 | 2019 Feb. 4T21:15:12 | NO | | | NO | |
| 1711 | 2019 Feb. 5T23:20:44 | NO | | | NO | |
| 1711 | 2019 Feb. 6T19:00:23 | NO | | | NO | |
| 1711 | 2019 Feb. 7T22:01:56 | NO | | | NO | |
| 1711 | 2019 Feb. 8T19:00:33 | NO | | | NO | |
| 1711 | 2019 Feb. 9T19:00:22 | NO | | | NO | |
| 1711 | 2019 Feb. 10T19:03:16 | NO | | | NO | |
| 1711 | 2019 Feb. 11T23:15:28 | NO | | | NO | |
| 1711 | 2019 Feb. 12T19:00:32 | NO | | | NO | |
| 1711 | 2019 Feb. 13T19:00:49 | NO | | | NO | |
| 1711 | 2019 Feb. 14T19:00:51 | NO | | | NO | |
| 1711 | 2019 Feb. 15T19:04:42 | NO | | | NO | |
| 1711 | 2019 Feb. 16T19:19:26 | NO | | | NO | |
| 1711 | 2019 Feb. 17T22:52:16 | NO | | | NO | |
| 1711 | 2019 Feb. 18T22:27:30 | YES | MILD | | YES | 1 |
| 1711 | 2019 Feb. 19T21:54:15 | NO | | | NO | |
| 1711 | 2019 Feb. 20T19:02:35 | NO | | | NO | |
| 1711 | 2019 Feb. 21T19:00:53 | NO | | | NO | |
| 1711 | 2019 Feb. 22T19:00:42 | NO | | | NO | |
| 1711 | 2019 Feb. 23T23:26:00 | NO | | | NO | |
| 1711 | 2019 Feb. 24T19:15:23 | NO | | | NO | |
| 1711 | 2019 Feb. 25T21:25:14 | NO | | | NO | |
| 1711 | 2019 Feb. 26T21:11:51 | YES | SEVERE | | YES | 1 |
| 1711 | 2019 Feb. 27T23:15:44 | NO | | | NO | |
| 1711 | 2019 Feb. 28T23:23:16 | NO | | | NO | |
| 1711 | 2019 Mar. 2T23:31:32 | NO | | | NO | |
| 1711 | 2019 Mar. 3T18:51:29 | NO | | | NO | |
| 1711 | 2019 Mar. 4T19:05:52 | NO | | | NO | |
| 1711 | 2019 Mar. 5T19:30:34 | NO | | | NO | |
| 1711 | 2019 Mar. 6T19:21:26 | NO | | | NO | |
| 1711 | 2019 Mar. 7T23:16:40 | NO | | | NO | |
| 1711 | 2019 Mar. 10T20:01:25 | NO | | | NO | |
| 1711 | 2019 Mar. 11T19:03:24 | NO | | | NO | |
| 1711 | 2019 Mar. 12T22:56:30 | NO | | | NO | |
| 1711 | 2019 Mar. 13T23:23:48 | NO | | | NO | |
| 1711 | 2019 Mar. 14T22:58:44 | YES | SEVERE | | YES | 1 |
| 1711 | 2019 Mar. 15T19:15:21 | NO | | | NO | |
| 1711 | 2019 Mar. 16T19:00:25 | NO | | | NO | |
| 1711 | 2019 Mar. 17T19:50:26 | NO | | | NO | |
| 1711 | 2019 Mar. 18T19:33:31 | NO | | | NO | |
| 1711 | 2019 Mar. 19T22:24:52 | NO | | | NO | |
| 1711 | 2019 Mar. 20T19:01:25 | NO | | | NO | |
| 1711 | 2019 Mar. 21T23:15:51 | NO | | | NO | |
| 1711 | 2019 Mar. 22T23:15:20 | NO | | | NO | |
| 1711 | 2019 Mar. 24T19:18:25 | NO | | | NO | |
| 1711 | 2019 Mar. 25T23:14:14 | NO | | | NO | |
| 1711 | 2019 Mar. 26T21:10:14 | NO | | | NO | |
| 1711 | 2019 Mar. 27T23:57:37 | NO | | | NO | |
| 1711 | 2019 Mar. 28T17:08:41 | NO | | | NO | |

Subject 1711 is the same as Subject D described above in the specification.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. A method for treating, preventing, alleviating or reducing the frequency of occurrence of headache in a patient in need thereof, comprising administering to the patient rimegepant, wherein said patient is concurrently undergoing treatment with a clostridial derivative.

2. The method according to claim 1, wherein the rimegepant is in the form of a hemisulfate sesquihydrate salt.

3. The method according to claim 1, wherein the clostridial derivative is botulinum toxin.

4. The method according to claim 1, wherein the headache is a migraine headache, a cluster headache, or a combination thereof.

5. A method for treating, preventing, alleviating or reducing the frequency of occurrence of migraine or a symptom of migraine in a patient in need thereof, comprising administering to the patient rimegepant, wherein said patient is concurrently undergoing treatment with a clostridial derivative.

6. The method according to claim 5, wherein the rimegepant is in the form of a hemisulfate sesquihydrate salt.

7. The method according to claim 5, wherein the clostridial derivative is botulinum toxin.

8. The method according to claim 5, wherein the patient experiences reduced severity of migraine after treatment with the rimegepant.

9. The method according to claim 5, wherein the migraine is breakthrough migraine.

10. A method for treating, preventing, alleviating or reducing the frequency of occurrence of headache in a patient in need thereof, comprising administering to the patient zavegepant, wherein said patient is concurrently undergoing treatment with a clostridial derivative.

11. The method according to claim 10, wherein the zavegepant is administered intranasally.

12. The method according to claim 10, wherein the clostridial derivative is botulinum toxin.

13. The method according to claim 10, wherein the patient experiences reduced severity of headache after treatment with the zavegepant.

14. The method according to claim 10, wherein the headache is a migraine headache, a cluster headache, or a combination thereof.

15. A method for treating, preventing, alleviating or reducing the frequency of occurrence of migraine or a symptom of migraine in a patient in need thereof, comprising administering to the patient zavegepant, wherein said patient is concurrently undergoing treatment with a clostridial derivative.

16. The method according to claim 15, wherein the zavegepant is administered intranasally.

17. The method according to claim 15, wherein the clostridial derivative is botulinum toxin.

18. The method according to claim 15, wherein the patient experiences reduced severity of migraine after treatment with the zavegepant.

19. The method according to claim 15, wherein the symptom of migraine is reduced after treatment with the CGRP antagonist.

20. The method according to claim 15, wherein the migraine is breakthrough migraine.
